SEQUENCE LISTING (311 sequences)

SEQ ID NO: 1 - protein, 758 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPAQDGRPPQTAAREATSIPGFPAEGAIPLPVDFLSKVSTEIPASEPDGPSVGRAKGQDAPLEFTFHVEITPNVQKEQAHSEEHLGRAAFPGAPGEGPEARGPSLGEDTKEADLPEPSEKQPAAAPRGKPVSRVPQLKARMVSKSKDGTGSDDKKAKTSTRSSAKTLKNRPCLSPKHPTPGSSDPLIQPSSPAVCPEPPSSPKYVSSVTSRTGSSGAKEMKLKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 2 - protein, 96 aa, Homo sapiens
DAKSLTAWSRTLVTFKDVFVDFTREEWKLLDTAQQIVYRNVMLENYKNLVSLGYQLTKPDVILRLEKGEEPWLVEREIHQETHPDSETAFEIKSSV

SEQ ID NO: 3 - protein, 5 aa, Artificial Sequence
  source: /note=“Description of Artificial Sequence: Synthetic peptide”
DGGGS

SEQ ID NO: 4 - protein, 5 aa, Artificial Sequence
  source: /note=“Description of Artificial Sequence: Synthetic peptide”
TGEKP

SEQ ID NO: 5 - protein, 9 aa, Artificial Sequence
  source: /note=“Description of Artificial Sequence: Synthetic peptide”
LRQKDGERP

SEQ ID NO: 6 - protein, 4 aa, Artificial Sequence
  source: /note=“Description of Artificial Sequence: Synthetic peptide”
GGRR

SEQ ID NO: 7 - protein, 8 aa, Artificial Sequence
  source: /note=“Description of Artificial Sequence: Synthetic peptide”
GGRRGGGS

SEQ ID NO: 8 - protein, 9 aa, Artificial Sequence
  source: /note=“Description of Artificial Sequence: Synthetic peptide”
LRQRDGERP

SEQ ID NO: 9 - protein, 12 aa, Artificial Sequence
  source: /note=“Description of Artificial Sequence: Synthetic peptide”
LRQKDGGGSERP

SEQ ID NO: 10 - protein, 16 aa, Artificial Sequence
  source: /note=“Description of Artificial Sequence: Synthetic peptide”
LRQKDGGGSGGGSERP

SEQ ID NO: 11 - protein, 6 aa, Artificial Sequence
  source: /note=“Description of Artificial Sequence: Synthetic peptide”
TGSQKP

SEQ ID NO: 12 - skipped

SEQ ID NO: 13 - protein, 10 aa, Artificial Sequence
  source: /note=“Description of Artificial Sequence: Synthetic peptide”
LRQKDAARGS

SEQ ID NO: 14 - protein, 12 aa, Artificial Sequence
  source: /note=“Description of Artificial Sequence: Synthetic peptide”
LRQKDAARGSGG

SEQ ID NO: 15 - protein, 20 aa, Artificial Sequence
  source: /note=“Description of Artificial Sequence: Synthetic peptide”
  SITE (1)..(20): /note=“This sequence may encompass 1-4 ′Gly Gly Gly Gly Ser′ repeating units”
GGGGSGGGGSGGGGSGGGGS

SEQ ID NO: 16 - DNA, 24 bp, Artificial Sequence
  source: /note=“Description of Artificial Sequence: Synthetic primer”
gcagagctctctggctaactagag

SEQ ID NO: 17 - DNA, 200 bp, Artificial Sequence
  source: /note=“Description of Artificial Sequence: Synthetic primer”
ttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttctggcaactagaaggcacag

SEQ ID NO: 18 - protein, 5 aa, Artificial Sequence
  source: /note=“Description of Artificial Sequence: Synthetic peptide”
GGGGS

SEQ ID NO: 19 - protein, 21 aa, Artificial Sequence
  source: /note=“Description of Artificial Sequence: Synthetic peptide”
GSGEGRGSLLTCGDVEENPGP

SEQ ID NO: 20 - protein, 7 aa, Artificial Sequence
  source: /note=“Description of Artificial Sequence: Synthetic peptide”
TGEKPFQ

SEQ ID NO: 21 - protein, 8 aa, Artificial Sequence
  source: /note=“Description of Artificial Sequence: Synthetic peptide”
TGGQRPFQ

SEQ ID NO: 22 - protein, 8 aa, Artificial Sequence
  source: /note=“Description of Artificial Sequence: Synthetic peptide”
TGSQKPFQ

SEQ ID NO: 23 - protein, 8 aa, Artificial Sequence
  source: /note=“Description of Artificial Sequence: Synthetic peptide”
TGSQRPFQ

SEQ ID NO: 24 - protein, 7 aa, Artificial Sequence
  source: /note=“Description of Artificial Sequence: Synthetic peptide”
TGEKPFA

SEQ ID NO: 25 - protein, 11 aa, Artificial Sequence
  source: /note=“Description of Artificial Sequence: Synthetic peptide”
TGGGGSQRPFQ

SEQ ID NO: 26 - protein, 11 aa, Artificial Sequence
  source: /note=“Description of Artificial Sequence: Synthetic peptide”
TGGGGSQKPFQ

SEQ ID NO: 27 - protein, 12 aa, Artificial Sequence
  source: /note=“Description of Artificial Sequence: Synthetic peptide”
THPRAPIPKPFQ

SEQ ID NO: 28 - protein, 12 aa, Artificial Sequence
  source: /note=“Description of Artificial Sequence: Synthetic peptide”
TPNRRPAPKPFQ

SEQ ID NO: 29 - protein, 12 aa, Artificial Sequence
  source: /note=“Description of Artificial Sequence: Synthetic peptide”
TVPRPTPPKPFQ

SEQ ID NO: 30 - protein, 12 aa, Artificial Sequence
  source: /note=“Description of Artificial Sequence: Synthetic peptide”
TYPRPIAAKPFQ

SEQ ID NO: 31 - protein, 14 aa, Artificial Sequence
  source: /note=“Description of Artificial Sequence: Synthetic peptide”
TGGGGSGGSQRPFQ

SEQ ID NO: 32 - protein, 14 aa, Artificial Sequence
  source: /note=“Description of Artificial Sequence: Synthetic peptide”
TGGGGSGGSQKPFQ

SEQ ID NO: 33 - protein, 14 aa, Artificial Sequence
  source: /note=“Description of Artificial Sequence: Synthetic peptide”
TLAPRPYRPPKPFQ

SEQ ID NO: 34 - protein, 16 aa, Artificial Sequence
  source: /note=“Description of Artificial Sequence: Synthetic peptide”
TPGGKSSRTDRNKPFQ

SEQ ID NO: 35 - protein, 16 aa, Artificial Sequence
  source: /note=“Description of Artificial Sequence: Synthetic peptide”
TPNPHRRTDPSHKPFQ

SEQ ID NO: 36 - protein, 6 aa, Artificial Sequence
  source: /note=“Description of Artificial Sequence: Synthetic peptide”
LRGSGG

SEQ ID NO: 37 - DNA, 16 bp, Homo sapiens
aggggcgggcagcgag

SEQ ID NO: 38 - DNA, 23 bp, Homo sapiens
ccgttgcgcctgattgatgccca

SEQ ID NO: 39 - DNA, 16 bp, Homo sapiens
cgacagaaggcgagga

SEQ ID NO: 40 - DNA, 22 bp, Homo sapiens
tggtgctggagctggtgggtgg

SEQ ID NO: 41 - DNA, 19 bp, Homo sapiens
gttccgtggcgcacgcgct

SEQ ID NO: 42 - DNA, 22 bp, Homo sapiens
gcgcgcacgcgcgttccgtggc

SEQ ID NO: 43 - DNA, 22 bp, Homo sapiens
gcggcgctgaccgcagcgcgca

SEQ ID NO: 44 - DNA, 22 bp, Homo sapiens
caggccgcggcgctgaccgcag

SEQ ID NO: 45 - DNA, 23 bp, Homo sapiens
cggccgagcggcagggcgctcgc

SEQ ID NO: 46 - DNA, 19 bp, Homo sapiens
aggcgcgagcagcgggaac

SEQ ID NO: 47 - DNA, 23 bp, Homo sapiens
gcggcgcaggcgcgagcagcggg

SEQ ID NO: 48 - DNA, 19 bp, Homo sapiens
gggcggcgcaggcgcgagc

SEQ ID NO: 49 - DNA, 22 bp, Homo sapiens
cgggcggcgcaggcgcgagcag

SEQ ID NO: 50 - DNA, 19 bp, Homo sapiens
gtgactgcgagggcgcgcg

SEQ ID NO: 51 - DNA, 22 bp, Homo sapiens
gtggcggtgactgcgagggcgc

SEQ ID NO: 52 - DNA, 23 bp, Homo sapiens
ggtggcggtgactgcgagggcgc

SEQ ID NO: 53 - DNA, 22 bp, Homo sapiens
tgggtggcggtgactgcgaggg

SEQ ID NO: 54 - DNA, 22 bp, Homo sapiens
tggtgggtggcggtgactgcga

SEQ ID NO: 55 - DNA, 23 bp, Homo sapiens
ctggtgggtggcggtgactgcga

SEQ ID NO: 56 - DNA, 22 bp, Homo sapiens
cggagctggtgggtggcggtga

SEQ ID NO: 57 - DNA, 22 bp, Homo sapiens
tggtgccggagctggtgggtgg

SEQ ID NO: 58 - DNA, 22 bp, Homo sapiens
ttggtgccggagctggtgggtg

SEQ ID NO: 59 - DNA, 22 bp, Homo sapiens
ctgctgttggtgccggagctgg

SEQ ID NO: 60 - DNA, 22 bp, Homo sapiens
gcgctgctgttggtgccggagc

SEQ ID NO: 61 - DNA, 22 bp, Homo sapiens
gcggcgctgctgttggtgccgg

SEQ ID NO: 62 - DNA, 22 bp, Homo sapiens
gcagcggcgctgctgttggtgc

SEQ ID NO: 63 - DNA, 23 bp, Homo sapiens
ggtggcagcggcgctgctgttgg

SEQ ID NO: 64 - DNA, 23 bp, Homo sapiens
agaaggtgggcggtggcagcggc

SEQ ID NO: 65 - DNA, 23 bp, Homo sapiens
cggcagaaggtgggcggtggcag

SEQ ID NO: 66 - DNA, 23 bp, Homo sapiens
ggcggcagaaggtgggcggtggc

SEQ ID NO: 67 - DNA, 23 bp, Homo sapiens
gtggcggcggcagaaggtgggcg

SEQ ID NO: 68 - DNA, 22 bp, Homo sapiens
gtggtggcggcggcagaaggtg

SEQ ID NO: 69 - DNA, 23 bp, Homo sapiens
gctgtggtggcggcggcagaagg

SEQ ID NO: 70 - DNA, 22 bp, Homo sapiens
gtggctgtggtggcggcggcag

SEQ ID NO: 71 - DNA, 23 bp, Homo sapiens
ggtggctgtggtggcggcggcag

SEQ ID NO: 72 - DNA, 22 bp, Homo sapiens
aaggtggctgtggtggcggcgg

SEQ ID NO: 73 - DNA, 22 bp, Homo sapiens
gagaaggtggctgtggtggcgg

SEQ ID NO: 74 - DNA, 22 bp, Homo sapiens
gaggagaaggtggctgtggtgg

SEQ ID NO: 75 - DNA, 23 bp, Homo sapiens
aggacgggagaggacagcggagg

SEQ ID NO: 76 - DNA, 22 bp, Homo sapiens
gaggcgaggacgggagaggaca

SEQ ID NO: 77 - DNA, 19 bp, Homo sapiens
cctctgtcgactatcaggt

SEQ ID NO: 78 - DNA, 23 bp, Homo sapiens
ctgtcgactatcaggtaagcgcc

SEQ ID NO: 79 - DNA, 23 bp, Homo sapiens
ggtaagcgccgcggctccgaaat

SEQ ID NO: 80 - DNA, 19 bp, Homo sapiens
cggacggcgaggcagattt

SEQ ID NO: 81 - DNA, 22 bp, Homo sapiens
aggcggacggcgaggcagattt

SEQ ID NO: 82 - DNA, 24 bp, Homo sapiens
gtgcacagaggcggacggcgaggc

SEQ ID NO: 83 - DNA, 22 bp, Homo sapiens
ggggcggcggcgcaggggtgca

SEQ ID NO: 84 - DNA, 23 bp, Homo sapiens
gcgaggggcggcggcgcaggggt

SEQ ID NO: 85 - DNA, 23 bp, Homo sapiens
agggcgaggggcggcggcgcagg

SEQ ID NO: 86 - DNA, 23 bp, Homo sapiens
gggagggcgaggggcggcggcgc

SEQ ID NO: 87 - DNA, 22 bp, Homo sapiens
gcgccgggcatcggtcggggcc

SEQ ID NO: 88 - DNA, 22 bp, Homo sapiens
gccagggcggcctggaaaggga

SEQ ID NO: 89 - protein, 7 aa, Artificial Sequence
  source: /note=“Description of Artificial Sequence: Synthetic peptide”
RSADLTR

SEQ ID NO: 90 - protein, 7 aa, Artificial Sequence
  source: /note=“Description of Artificial Sequence: Synthetic peptide”
ERGTLAR

SEQ ID NO: 91 - protein, 7 aa, Artificial Sequence
  source: /note=“Description of Artificial Sequence: Synthetic peptide”
RSDNLAR

SEQ ID NO: 92 - protein, 7 aa, Artificial Sequence
  source: /note=“Description of Artificial Sequence: Synthetic peptide”
LRHHLTR

SEQ ID NO: 93 - protein, 7 aa, Artificial Sequence
  source: /note=“Description of Artificial Sequence: Synthetic peptide”
RRSDLKR

SEQ ID NO: 94 - protein, 7 aa, Artificial Sequence
  source: /note=“Description of Artificial Sequence: Synthetic peptide”
RSDSLSR

SEQ ID NO: 95 - protein, 7 aa, Artificial Sequence
  source: /note=“Description of Artificial Sequence: Synthetic peptide”
RSDDLTR

SEQ ID NO: 96 - protein, 7 aa, Artificial Sequence
  source: /note=“Description of Artificial Sequence: Synthetic peptide”
QAHHLRT

SEQ ID NO: 97 - protein, 7 aa, Artificial Sequence
  source: /note=“Description of Artificial Sequence: Synthetic peptide”
DRSYRNT

SEQ ID NO: 98 - protein, 7 aa, Artificial Sequence
  source: /note=“Description of Artificial Sequence: Synthetic peptide”
QSGHLSR

SEQ ID NO: 99 - protein, 7 aa, Artificial Sequence
  source: /note=“Description of Artificial Sequence: Synthetic peptide”
QNAHRKT

SEQ ID NO: 100 - protein, 7 aa, Artificial Sequence
  source: /note=“Description of Artificial Sequence: Synthetic peptide”
MSCCRSA

SEQ ID NO: 101 - protein, 7 aa, Artificial Sequence
  source: /note=“Description of Artificial Sequence: Synthetic peptide”
DSSHRTR

SEQ ID NO: 102 - protein, 7 aa, Artificial Sequence
  source: /note=“Description of Artificial Sequence: Synthetic peptide”
SSGHLPP

SEQ ID NO: 103 - protein, 7 aa, Artificial Sequence
  source: /note=“Description of Artificial Sequence: Synthetic peptide”
TSGHLSR

SEQ ID NO: 104 - protein, 7 aa, Artificial Sequence
  source: /note=“Description of Artificial Sequence: Synthetic peptide”
RSAHLSR

SEQ ID NO: 105 - protein, 7 aa, Artificial Sequence
  source: /note=“Description of Artificial Sequence: Synthetic peptide”
QSSDLSR

SEQ ID NO: 106 - protein, 7 aa, Artificial Sequence
  source: /note=“Description of Artificial Sequence: Synthetic peptide”
QSGHLAR

SEQ ID NO: 107 - protein, 7 aa, Artificial Sequence
  source: /note=“Description of Artificial Sequence: Synthetic peptide”
TSGSLSR

SEQ ID NO: 108 - protein, 7 aa, Artificial Sequence
  source: /note=“Description of Artificial Sequence: Synthetic peptide”
RSDDLSK

SEQ ID NO: 109 - protein, 7 aa, Artificial Sequence
  source: /note=“Description of Artificial Sequence: Synthetic peptide”
DRSHLTR

SEQ ID NO: 110 - protein, 7 aa, Artificial Sequence
  source: /note=“Description of Artificial Sequence: Synthetic peptide”
RSDHLSQ

SEQ ID NO: 111 - protein, 7 aa, Artificial Sequence
  source: /note=“Description of Artificial Sequence: Synthetic peptide”
QSGNLAR

SEQ ID NO: 112 - protein, 7 aa, Artificial Sequence
  source: /note=“Description of Artificial Sequence: Synthetic peptide”
DRSHLSR

SEQ ID NO: 113 - protein, 7 aa, Artificial Sequence
  source: /note=“Description of Artificial Sequence: Synthetic peptide”
RSDNLSE

SEQ ID NO: 114 - protein, 7 aa, Artificial Sequence
  source: /note=“Description of Artificial Sequence: Synthetic peptide”
QSSNLAR

SEQ ID NO: 115 - protein, 7 aa, Artificial Sequence
  source: /note=“Description of Artificial Sequence: Synthetic peptide”
TSGNLTR

SEQ ID NO: 116 - protein, 7 aa, Artificial Sequence
  source: /note=“Description of Artificial Sequence: Synthetic peptide”
RSANLAR

SEQ ID NO: 117 - protein, 7 aa, Artificial Sequence
  source: /note=“Description of Artificial Sequence: Synthetic peptide”
RSDSLSA

SEQ ID NO: 118 - protein, 7 aa, Artificial Sequence
  source: /note=“Description of Artificial Sequence: Synthetic peptide”
RSDHLSK

SEQ ID NO: 119 - protein, 7 aa, Artificial Sequence
  source: /note=“Description of Artificial Sequence: Synthetic peptide”
QSGDLTR

SEQ ID NO: 120 - protein, 7 aa, Artificial Sequence
  source: /note=“Description of Artificial Sequence: Synthetic peptide”
RSDHLSN

SEQ ID NO: 121 - protein, 7 aa, Artificial Sequence
  source: /note=“Description of Artificial Sequence: Synthetic peptide”
RSDNLSN

SEQ ID NO: 122 - protein, 7 aa, Artificial Sequence
  source: /note=“Description of Artificial Sequence: Synthetic peptide”
TSANLSR

SEQ ID NO: 123 - protein, 7 aa, Artificial Sequence
  source: /note=“Description of Artificial Sequence: Synthetic peptide”
DRSHLAR

SEQ ID NO: 124 - protein, 7 aa, Artificial Sequence
  source: /note=“Description of Artificial Sequence: Synthetic peptide”
RRFTLSK

SEQ ID NO: 125 - protein, 7 aa, Artificial Sequence
  source: /note=“Description of Artificial Sequence: Synthetic peptide”
DQSNLRA

SEQ ID NO: 126 - protein, 7 aa, Artificial Sequence
  source: /note=“Description of Artificial Sequence: Synthetic peptide”
DRSDRTK

SEQ ID NO: 127 - protein, 7 aa, Artificial Sequence
  source: /note=“Description of Artificial Sequence: Synthetic peptide”
MSYQLGR

SEQ ID NO: 128 - protein, 7 aa, Artificial Sequence
  source: /note=“Description of Artificial Sequence: Synthetic peptide”
DRSNLTR

SEQ ID NO: 129 - protein, 7 aa, Artificial Sequence
  source: /note=“Description of Artificial Sequence: Synthetic peptide”
QAATLSR

SEQ ID NO: 130 - protein, 7 aa, Artificial Sequence
  source: /note=“Description of Artificial Sequence: Synthetic peptide”
RSADRKK

SEQ ID NO: 131 - protein, 7 aa, Artificial Sequence
  source: /note=“Description of Artificial Sequence: Synthetic peptide”
RSADLSR

SEQ ID NO: 132 - protein, 7 aa, Artificial Sequence
  source: /note=“Description of Artificial Sequence: Synthetic peptide”
MQNYLSR

SEQ ID NO: 133 - protein, 7 aa, Artificial Sequence
  source: /note=“Description of Artificial Sequence: Synthetic peptide”
FREGLYK

SEQ ID NO: 134 - protein, 7 aa, Artificial Sequence
  source: /note=“Description of Artificial Sequence: Synthetic peptide”
CPKCRNK

SEQ ID NO: 135 - protein, 7 aa, Artificial Sequence
  source: /note=“Description of Artificial Sequence: Synthetic peptide”
TSGSLTR

SEQ ID NO: 136 - protein, 7 aa, Artificial Sequence
  source: /note=“Description of Artificial Sequence: Synthetic peptide”
HRSARKR

SEQ ID NO: 137 - protein, 7 aa, Artificial Sequence
  source: /note=“Description of Artificial Sequence: Synthetic peptide”
HSSDLNR

SEQ ID NO: 138 - protein, 7 aa, Artificial Sequence
  source: /note=“Description of Artificial Sequence: Synthetic peptide”
QSATRTK

SEQ ID NO: 139 - protein, 7 aa, Artificial Sequence
  source: /note=“Description of Artificial Sequence: Synthetic peptide”
LKQHLTR

SEQ ID NO: 140 - protein, 7 aa, Artificial Sequence
  source: /note=“Description of Artificial Sequence: Synthetic peptide”
MACCRYA

SEQ ID NO: 141 - protein, 7 aa, Artificial Sequence
  source: /note=“Description of Artificial Sequence: Synthetic peptide”
QNAHRIT

SEQ ID NO: 142 - protein, 7 aa, Artificial Sequence
  source: /note=“Description of Artificial Sequence: Synthetic peptide”
HKQHRDA

SEQ ID NO: 143 - protein, 7 aa, Artificial Sequence
  source: /note=“Description of Artificial Sequence: Synthetic peptide”
SPCCRYH

SEQ ID NO: 144 - protein, 7 aa, Artificial Sequence
  source: /note=“Description of Artificial Sequence: Synthetic peptide”
QLTHLNS

SEQ ID NO: 145 - protein, 7 aa, Artificial Sequence
  source: /note=“Description of Artificial Sequence: Synthetic peptide”
TSSNRKT

SEQ ID NO: 146 - protein, 7 aa, Artificial Sequence
  source: /note=“Description of Artificial Sequence: Synthetic peptide”
QSANRTK

SEQ ID NO: 147 - protein, 7 aa, Artificial Sequence
  source: /note=“Description of Artificial Sequence: Synthetic peptide”
DSSDRKK

SEQ ID NO: 148 - protein, 7 aa, Artificial Sequence
  source: /note=“Description of Artificial Sequence: Synthetic peptide”
QQNTLTM

SEQ ID NO: 149 - protein, 7 aa, Artificial Sequence
  source: /note=“Description of Artificial Sequence: Synthetic peptide”
RSDHLSR

SEQ ID NO: 150 - protein, 7 aa, Artificial Sequence
  source: /note=“Description of Artificial Sequence: Synthetic peptide”
QNATRTK

SEQ ID NO: 151 - protein, 7 aa, Artificial Sequence
  source: /note=“Description of Artificial Sequence: Synthetic peptide”
TQGYLRK

SEQ ID NO: 152 - protein, 7 aa, Artificial Sequence
  source: /note=“Description of Artificial Sequence: Synthetic peptide”
RSDDRKK

SEQ ID NO: 153 - protein, 7 aa, Artificial Sequence
  source: /note=“Description of Artificial Sequence: Synthetic peptide”
TSSSRIN

SEQ ID NO: 154 - protein, 7 aa, Artificial Sequence
  source: /note=“Description of Artificial Sequence: Synthetic peptide”
RSDHLSE

SEQ ID NO: 155 - protein, 7 aa, Artificial Sequence
  source: /note=“Description of Artificial Sequence: Synthetic peptide”
RSDVLSE

SEQ ID NO: 156 - protein, 7 aa, Artificial Sequence
  source: /note=“Description of Artificial Sequence: Synthetic peptide”
RKSDRIK

SEQ ID NO: 157 - protein, 7 aa, Artificial Sequence
  source: /note=“Description of Artificial Sequence: Synthetic peptide”
RKQDLRT

SEQ ID NO: 158 - protein, 7 aa, Artificial Sequence
  source: /note=“Description of Artificial Sequence: Synthetic peptide”
RSDNLSQ

SEQ ID NO: 159 - protein, 7 aa, Artificial Sequence
  source: /note=“Description of Artificial Sequence: Synthetic peptide”
RSDNLSA

SEQ ID NO: 160 - protein, 7 aa, Artificial Sequence
  source: /note=“Description of Artificial Sequence: Synthetic peptide”
RSANLTR

SEQ ID NO: 161 - protein, 7 aa, Artificial Sequence
  source: /note=“Description of Artificial Sequence: Synthetic peptide”
DRSNLSR

SEQ ID NO: 162 - protein, 7 aa, Artificial Sequence
  source: /note=“Description of Artificial Sequence: Synthetic peptide”
RSDVLSK

SEQ ID NO: 163 - protein, 7 aa, Artificial Sequence
  source: /note=“Description of Artificial Sequence: Synthetic peptide”
RQDCLSL

SEQ ID NO: 164 - protein, 7 aa, Artificial Sequence
  source: /note=“Description of Artificial Sequence: Synthetic peptide”
WRSCRSA

SEQ ID NO: 165 - protein, 7 aa, Artificial Sequence
  source: /note=“Description of Artificial Sequence: Synthetic peptide”
RSDNLTR

SEQ ID NO: 166 - protein, 7 aa, Artificial Sequence
  source: /note=“Description of Artificial Sequence: Synthetic peptide”
RSDHLST

SEQ ID NO: 167 - protein, 7 aa, Artificial Sequence
  source: /note=“Description of Artificial Sequence: Synthetic peptide”
RSDALSV

SEQ ID NO: 168 - protein, 7 aa, Artificial Sequence
  source: /note=“Description of Artificial Sequence: Synthetic peptide”
HRTSLTD

SEQ ID NO: 169 - protein, 7 aa, Artificial Sequence
  source: /note=“Description of Artificial Sequence: Synthetic peptide”
QSNTRIM

SEQ ID NO: 170 - protein, 7 aa, Artificial Sequence
  source: /note=“Description of Artificial Sequence: Synthetic peptide”
KHSTRRV

SEQ ID NO: 171 - protein, 7 aa, Artificial Sequence
  source: /note=“Description of Artificial Sequence: Synthetic peptide”
RSNARTN

SEQ ID NO: 172 - protein, 7 aa, Artificial Sequence
  source: /note=“Description of Artificial Sequence: Synthetic peptide”
RSNDRKK

SEQ ID NO: 173 - protein, 7 aa, Artificial Sequence
  source: /note=“Description of Artificial Sequence: Synthetic peptide”
HRSTRNR

SEQ ID NO: 174 - protein, 7 aa, Artificial Sequence
  source: /note=“Description of Artificial Sequence: Synthetic peptide”
RSDDRIK

SEQ ID NO: 175 - protein, 7 aa, Artificial Sequence
  source: /note=“Description of Artificial Sequence: Synthetic peptide”
RNDNRKT

SEQ ID NO: 176 - protein, 7 aa, Artificial Sequence
  source: /note=“Description of Artificial Sequence: Synthetic peptide”
DSSARNT

SEQ ID NO: 177 - protein, 7 aa, Artificial Sequence
  source: /note=“Description of Artificial Sequence: Synthetic peptide”
RNRDRIT

SEQ ID NO: 178 - protein, 7 aa, Artificial Sequence
  source: /note=“Description of Artificial Sequence: Synthetic peptide”
TNSNRIT

SEQ ID NO: 179 - protein, 7 aa, Artificial Sequence
  source: /note=“Description of Artificial Sequence: Synthetic peptide”
RSHSLLR

SEQ ID NO: 180 - protein, 7 aa, Artificial Sequence
  source: /note=“Description of Artificial Sequence: Synthetic peptide”
YDYGRYT

SEQ ID NO: 181 - protein, 7 aa, Artificial Sequence
  source: /note=“Description of Artificial Sequence: Synthetic peptide”
DDWNLSQ

SEQ ID NO: 182 - protein, 7 aa, Artificial Sequence
  source: /note=“Description of Artificial Sequence: Synthetic peptide”
RSDTLSE

SEQ ID NO: 183 - protein, 7 aa, Artificial Sequence
  source: /note=“Description of Artificial Sequence: Synthetic peptide”
RSDNLST

SEQ ID NO: 184 - protein, 7 aa, Artificial Sequence
  source: /note=“Description of Artificial Sequence: Synthetic peptide”
RNSDRTK

SEQ ID NO: 185 - protein, 7 aa, Artificial Sequence
  source: /note=“Description of Artificial Sequence: Synthetic peptide”
RSDALSE

SEQ ID NO: 186 - protein, 7 aa, Artificial Sequence
  source: /note=“Description of Artificial Sequence: Synthetic peptide”
HPSARKR

SEQ ID NO: 187 - protein, 7 aa, Artificial Sequence
  source: /note=“Description of Artificial Sequence: Synthetic peptide”
RLYTLHK

SEQ ID NO: 188 - protein, 7 aa, Artificial Sequence
  source: /note=“Description of Artificial Sequence: Synthetic peptide”
DRSVLHR

SEQ ID NO: 189 - protein, 7 aa, Artificial Sequence
  source: /note=“Description of Artificial Sequence: Synthetic peptide”
RNQHRKN

SEQ ID NO: 190 - protein, 7 aa, Artificial Sequence
  source: /note=“Description of Artificial Sequence: Synthetic peptide”
HHSALRV

SEQ ID NO: 191 - protein, 7 aa, Artificial Sequence
  source: /note=“Description of Artificial Sequence: Synthetic peptide”
NSRNLRN

SEQ ID NO: 192 - protein, 7 aa, Artificial Sequence
  source: /note=“Description of Artificial Sequence: Synthetic peptide”
FHSCLSA

SEQ ID NO: 193 - protein, 7 aa, Artificial Sequence
  source: /note=“Description of Artificial Sequence: Synthetic peptide”
RNQHRKT

SEQ ID NO: 194 - protein, 7 aa, Artificial Sequence
  source: /note=“Description of Artificial Sequence: Synthetic peptide”
DAFTRTR

SEQ ID NO: 195 - protein, 7 aa, Artificial Sequence
  source: /note=“Description of Artificial Sequence: Synthetic peptide”
DRSDLSR

SEQ ID NO: 196 - protein, 7 aa, Artificial Sequence
  source: /note=“Description of Artificial Sequence: Synthetic peptide”
AKWNLDA

SEQ ID NO: 197 - protein, 237 aa, Artificial Sequence
  source: /note=“Description of Artificial Sequence: Synthetic polypeptide”
MAPKKKRKVGVPAAMAERPFQCRICMRNFSRSADLTRHIRTHTGEKPFACDICGRKFAQSGDLTRHTKIHTGSQKPFQCRICMRNFSRSDHLSEHIRTHTGEKPFACDICGRKFARSAHLSRHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 198 - protein, 298 aa, Artificial Sequence
  source: /note=“Description of Artificial Sequence: Synthetic polypeptide”
MAPKKKRKVGVPAAMAERPFQCRICMRNFSERGTLARHIRTHTGEKPFACDICGRKFATSANLSRHTKIHTHPRAPIPKPFQCRICMRNFSTSGNLTRHIRTHTGEKPFACDICGRKFAHRTSLTDHTKIHTGSQKPFQCRICMRNFSRSHSLLRHIRTHTGEKPFACDICGRKFAHPSARKRHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 199 - protein, 237 aa, Artificial Sequence
  source: /note=“Description of Artificial Sequence: Synthetic polypeptide”
MAPKKKRKVGVPAAMAERPFQCRICMRNFSRSDNLARHIRTHTGEKPFACDICGRKFADRSHLARHTKIHTGSQKPFQCRICMRNFSQSGNLARHIRTHTGEKPFACDICGRKFAQSNTRIMHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 200 - protein, 294 aa, Artificial Sequence
  source: /note=“Description of Artificial Sequence: Synthetic polypeptide”
MAPKKKRKVGVPAAMAERPFQCRICMRNFSLRHHLTRHIRTHTGEKPFACDICGRKFARRFTLSKHTKIHTGSQKPFQCRICMRNFSRSDVLSEHIRTHTGEKPFACDICGRKFAKHSTRRVHTKIHTGSQKPFQCRICMRNFSRSDVLSEHIRTHTGEKPFACDICGRKFARLYTLHKHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 201 - protein, 265 aa, Artificial Sequence
  source: /note=“Description of Artificial Sequence: Synthetic polypeptide”
MAPKKKRKVGVPAAMAERPFQCRICMRKFARRSDLKRHTKIHTGEKPFQCRICMRNFSDQSNLRAHIRTHTGEKPFACDICGRKFARKSDRIKHTKIHTGSQKPFQCRICMRNFSRSDSLSRHIRTHTGEKPFACDICGKKFADRSDRTKHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 202 - protein, 294 aa, Artificial Sequence
  source: /note=“Description of Artificial Sequence: Synthetic polypeptide”
MAPKKKRKVGVPAAMAERPFQCRICMRNFSRSDSLSRHIRTHTGEKPFACDICGKKFADRSDRTKHTKIHTGSQKPFQCRICMRNFSRKQTRTTHIRTHTGEKPFACDICGRKFARRSDLKRHTKIHTGSQKPFQCRICMRNFSDQSNLRAHIRTHTGEKPFACDICGRKFARRSDLKRHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 203 - protein, 294 aa, Artificial Sequence
  source: /note=“Description of Artificial Sequence: Synthetic polypeptide”
MAPKKKRKVGVPAAMAERPFQCRICMRNFSRSDDLTRHIRTHTGEKPFACDICGRKFAMSYQLGRHTKIHTGSQKPFQCRICMRNFSQAHHLRTHIRTHTGEKPFACDICGRKFADRSNLTRHTKIHTGSQKPFQCRICMRNFSQSSDLSRHIRTHTGEKPFACDICGRKFADRSHLARHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 204 - protein, 294 aa, Artificial Sequence
  source: /note=“Description of Artificial Sequence: Synthetic polypeptide”
MAPKKKRKVGVPAAMAERPFQCRICMRNFSQAHHLRTHIRTHTGEKPFACDICGRKFADRSNLTRHTKIHTGSQKPFQCRICMRNFSQSSDLSRHIRTHTGEKPFACDICGRKFADRSHLARHTKIHTGSQKPFQCRICMRNFSYDYGRYTHIRTHTGEKPFACDICGRKFADRSHLARHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 205 - protein, 298 aa, Artificial Sequence
  source: /note=“Description of Artificial Sequence: Synthetic polypeptide”
MAPKKKRKVGVPAAMAERPFQCRICMRNFSDRSYRNTHIRTHTGEKPFACDICGRKFARRSDLKRHTKIHTHPRAPIPKPFQCRICMRNFSRSDNLSQHIRTHTGEKPFACDICGRKFARSNARTNHTKIHTGSQKPFQCRICMRNFSRSDNLARHIRTHTGEKPFACDICGRKFADRSVLHRHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 206 - protein, 265 aa, Artificial Sequence
  source: /note=“Description of Artificial Sequence: Synthetic polypeptide”
MAPKKKRKVGVPAAMAERPFQCRICMRKFAQSGHLSRHTKIHTGEKPFQCRICMRNFSRSADLTRHIRTHTGEKPFACDICGRKFAQSGDLTRHTKIHTGSQKPFQCRICMRNFSQNAHRKTHIRTHTGEKPFACDICGRKFARRSDLKRHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 207 - protein, 298 aa, Artificial Sequence
  source: /note=“Description of Artificial Sequence: Synthetic polypeptide”
MAPKKKRKVGVPAAMAERPFQCRICMRNFSRSDDLTRHIRTHTGEKPFACDICGRKFAQAATLSRHTKIHTGSQKPFQCRICMRNFSQNAHRKTHIRTHTGEKPFACDICGRKFARRSDLKRHTKIHTHPRAPIPKPFQCRICMRNFSQSGDLTRHIRTHTGEKPFACDICGRKFATQGYLRKHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 208 - protein, 265 aa, Artificial Sequence
  source: /note=“Description of Artificial Sequence: Synthetic polypeptide”
MAPKKKRKVGVPAAMAERPFQCRICMRNFSQNAHRKTHIRTHTGEKPFACDICGRKFARSADRKKHTKIHTGSQKPFQCRICMRNFSRSDNLSAHIRTHTGEKPFACDICGRKFARSNDRKKHTKIHTGEKPFQCRICMRKFARSDDLTRHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 209 - protein, 294 aa, Artificial Sequence
  source: /note=“Description of Artificial Sequence: Synthetic polypeptide”
MAPKKKRKVGVPAAMAERPFQCRICMRNFSMSCCRSAHIRTHTGEKPFACDICGRKFARSADLSRHTKIHTGSQKPFQCRICMRNFSDRSHLTRHIRTHTGEKPFACDICGRKFAQSGDLTRHTKIHTGSQKPFQCRICMRNFSDRSHLSRHIRTHTGEKPFACDICGRKFADRSHLARHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 210 - protein, 265 aa, Artificial Sequence
  source: /note=“Description of Artificial Sequence: Synthetic polypeptide”
MAPKKKRKVGVPAAMAERPFQCRICMRNFSRSADLTRHIRTHTGEKPFACDICGRKFADRSHLARHTKIHTGSQKPFQCRICMRKFARSANLTRHTKIHTGEKPFQCRICMRNFSSSGHLPPHIRTHTGEKPFACDICGRKFADRSNLTRHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 211 - protein, 294 aa, Artificial Sequence
  source: /note=“Description of Artificial Sequence: Synthetic polypeptide”
MAPKKKRKVGVPAAMAERPFQCRICMRNFSDSSHRTRHIRTHTGEKPFACDICGRKFAMQNYLSRHTKIHTGSQKPFQCRICMRNFSSSGHLPPHIRTHTGEKPFACDICGRKFADRSNLTRHTKIHTGSQKPFQCRICMRNFSTSGHLSRHIRTHTGEKPFACDICGRKFADRSHLARHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 212 - protein, 298 aa, Artificial Sequence
  source: /note=“Description of Artificial Sequence: Synthetic polypeptide”
MAPKKKRKVGVPAAMAERPFQCRICMRNFSDSSHRTRHIRTHTGEKPFACDICGRKFAMQNYLSRHTKIHTGSQKPFQCRICMRNFSSSGHLPPHIRTHTGEKPFACDICGRKFADRSNLTRHTKIHTHPRAPIPKPFQCRICMRNFSRSDHLSEHIRTHTGEKPFACDICGKKFARNQHRKNHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 213 - protein, 294 aa, Artificial Sequence
  source: /note=“Description of Artificial Sequence: Synthetic polypeptide”
MAPKKKRKVGVPAAMAERPFQCRICMRNFSRSDNLARHIRTHTGEKPFACDICGRKFAFREGLYKHTKIHTGSQKPFQCRICMRNFSDRSNLSRHIRTHTGEKPFACDICGRKFALKQHLTRHTKIHTGSQKPFQCRICMRNFSDRSHLTRHIRTHTGEKPFACDICGRKFALKQHLTRHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 214 - protein, 294 aa, Artificial Sequence
  source: /note=“Description of Artificial Sequence: Synthetic polypeptide”
MAPKKKRKVGVPAAMAERPFQCRICMRNFSSSGHLPPHIRTHTGEKPFACDICGRKFADRSNLTRHTKIHTGSQKPFQCRICMRNFSTSGHLSRHIRTHTGEKPFACDICGRKFADRSHLARHTKIHTGSQKPFQCRICMRNFSLRHHLTRHIRTHTGEKPFACDICGRKFARRFTLSKHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 215 - protein, 298 aa, Artificial Sequence
  source: /note=“Description of Artificial Sequence: Synthetic polypeptide”
MAPKKKRKVGVPAAMAERPFQCRICMRNFSSSGHLPPHIRTHTGEKPFACDICGRKFADRSNLTRHTKIHTGSQKPFQCRICMRNFSTSGHLSRHIRTHTGEKPFACDICGRKFADRSHLARHTKIHTHPRAPIPKPFQCRICMRNFSRSAHLSRHIRTHTGEKPFACDICGRKFATSGHLSRHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 216 - protein, 294 aa, Artificial Sequence
  source: /note=“Description of Artificial Sequence: Synthetic polypeptide”
MAPKKKRKVGVPAAMAERPFQCRICMRNFSTSGHLSRHIRTHTGEKPFACDICGRKFADRSHLARHTKIHTGSQKPFQCRICMRNFSLRHHLTRHIRTHTGEKPFACDICGRKFARRFTLSKHTKIHTGSQKPFQCRICMRNFSDDWNLSQHIRTHTGEKPFACDICGRKFARSANLTRHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 217 - protein, 294 aa, Artificial Sequence
  source: /note=“Description of Artificial Sequence: Synthetic polypeptide”
MAPKKKRKVGVPAAMAERPFQCRICMRNFSLRHHLTRHIRTHTGEKPFACDICGRKFARRFTLSKHTKIHTGSQKPFQCRICMRNFSRSDVLSEHIRTHTGEKPFACDICGRKFAKHSTRRVHTKIHTGSQKPFQCRICMRNFSRSDTLSEHIRTHTGEKPFACDICGRKFAHHSALRVHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 218 - protein, 294 aa, Artificial Sequence
  source: /note=“Description of Artificial Sequence: Synthetic polypeptide”
MAPKKKRKVGVPAAMAERPFQCRICMRNFSRSAHLSRHIRTHTGEKPFACDICGRKFATSGHLSRHTKIHTGSQKPFQCRICMRNFSQSSDLSRHIRTHTGEKPFACDICGRKFAQSGHLSRHTKIHTGSQKPFQCRICMRNFSERGTLARHIRTHTGEKPFACDICGRKFATSGSLTRHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 219 - protein, 294 aa, Artificial Sequence
  source: /note=“Description of Artificial Sequence: Synthetic polypeptide”
MAPKKKRKVGVPAAMAERPFQCRICMRNFSQSSDLSRHIRTHTGEKPFACDICGRKFAQSGHLSRHTKIHTGSQKPFQCRICMRNFSERGTLARHIRTHTGEKPFACDICGRKFATSGSLTRHTKIHTGSQKPFQCRICMRNFSTSGSLSRHIRTHTGEKPFACDICGRKFAHSSDLNRHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 220 - protein, 294 aa, Artificial Sequence
  source: /note=“Description of Artificial Sequence: Synthetic polypeptide”
MAPKKKRKVGVPAAMAERPFQCRICMRNFSQSGHLARHIRTHTGEKPFACDICGRKFACPKCRNKHTKIHTGSQKPFQCRICMRNFSLRHHLTRHIRTHTGEKPFACDICGRKFAHRSARKRHTKIHTGSQKPFQCRICMRNFSQSSDLSRHIRTHTGEKPFACDICGRKFAHRSTRNRHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 221 - protein, 294 aa, Artificial Sequence
  source: /note=“Description of Artificial Sequence: Synthetic polypeptide”
MAPKKKRKVGVPAAMAERPFQCRICMRNFSERGTLARHIRTHTGEKPFACDICGRKFATSGSLTRHTKIHTGSQKPFQCRICMRNFSTSGSLSRHIRTHTGEKPFACDICGRKFAHSSDLNRHTKIHTGSQKPFQCRICMRNFSQSSDLSRHIRTHTGEKPFACDICGRKFADRSHLARHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 222 - protein, 294 aa, Artificial Sequence
  source: /note=“Description of Artificial Sequence: Synthetic polypeptide”
MAPKKKRKVGVPAAMAERPFQCRICMRNFSLRHHLTRHIRTHTGEKPFACDICGRKFAHRSARKRHTKIHTGSQKPFQCRICMRNFSQSSDLSRHIRTHTGEKPFACDICGRKFAHRSTRNRHTKIHTGSQKPFQCRICMRNFSDRSHLTRHIRTHTGEKPFACDICGRKFANSRNLRNHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 223 - protein, 298 aa, Artificial Sequence
  source: /note=“Description of Artificial Sequence: Synthetic polypeptide”
MAPKKKRKVGVPAAMAERPFQCRICMRNFSTSGSLSRHIRTHTGEKPFACDICGRKFAHSSDLNRHTKIHTGSQKPFQCRICMRNFSQSSDLSRHIRTHTGEKPFACDICGRKFADRSHLARHTKIHTHPRAPIPKPFQCRICMRNFSRSDNLSTHIRTHTGEKPFACDICGRKFAFHSCLSAHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 224 - protein, 298 aa, Artificial Sequence
  source: /note=“Description of Artificial Sequence: Synthetic polypeptide”
MAPKKKRKVGVPAAMAERPFQCRICMRNFSRSDDLSKHIRTHTGEKPFACDICGRKFAQSATRTKHTKIHTGSQKPFQCRICMRNFSRSDVLSKHIRTHTGEKPFACDICGRKFARSDDRIKHTKIHTHPRAPIPKPFQCRICMRNFSRQDCLSLHIRTHTGEKPFACDICGRKFARNDNRKTHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 225 - protein, 298 aa, Artificial Sequence
  source: /note=“Description of Artificial Sequence: Synthetic polypeptide”
MAPKKKRKVGVPAAMAERPFQCRICMRNFSDRSHLTRHIRTHTGEKPFACDICGRKFALKQHLTRHTKIHTHPRAPIPKPFQCRICMRNFSRSAHLSRHIRTHTGEKPFACDICGRKFATSGHLSRHTKIHTGSQKPFQCRICMRNFSQSGNLARHIRTHTGEKPFACDICGRKFAQSGDLTRHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 226 - protein, 298 aa, Artificial Sequence
  source: /note=“Description of Artificial Sequence: Synthetic polypeptide”
MAPKKKRKVGVPAAMAERPFQCRICMRNFSRSDVLSKHIRTHTGEKPFACDICGRKFARSDDRIKHTKIHTHPRAPIPKPFQCRICMRNFSRSDVLSEHIRTHTGEKPFACDICGRKFAKRWNRQQHTKIHTGSQKPFQCRICMRNFSRSDNLSQHIRTHTGEKPFACDICGRKFARSNARTNHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 227 - protein, 298 aa, Artificial Sequence
  source: /note=“Description of Artificial Sequence: Synthetic polypeptide”
MAPKKKRKVGVPAAMAERPFQCRICMRNFSRSAHLSRHIRTHTGEKPFACDICGRKFATSGHLSRHTKIHTGSQKPFQCRICMRNFSQSGNLARHIRTHTGEKPFACDICGRKFAQSGDLTRHTKIHTHPRAPIPKPFQCRICMRNFSDRSHLSRHIRTHTGEKPFACDICGRKFADRSHLARHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 228 - protein, 294 aa, Artificial Sequence
  source: /note=“Description of Artificial Sequence: Synthetic polypeptide”
MAPKKKRKVGVPAAMAERPFQCRICMRNFSRSDHLSQHIRTHTGEKPFACDICGRKFAQNAHRITHTKIHTGSQKPFQCRICMRNFSDRSHLSRHIRTHTGEKPFACDICGRKFADRSHLARHTKIHTGSQKPFQCRICMRNFSDRSHLTRHIRTHTGEKPFACDICGRKFALKQHLTRHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 229 - protein, 298 aa, Artificial Sequence
  source: /note=“Description of Artificial Sequence: Synthetic polypeptide”
MAPKKKRKVGVPAAMAERPFQCRICMRNFSQSGNLARHIRTHTGEKPFACDICGRKFAQSGDLTRHTKIHTHPRAPIPKPFQCRICMRNFSDRSHLSRHIRTHTGEKPFACDICGRKFADRSHLARHTKIHTGSQKPFQCRICMRNFSLRHHLTRHIRTHTGEKPFACDICGRKFAHKQHRDAHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 230 - protein, 294 aa, Artificial Sequence
  source: /note=“Description of Artificial Sequence: Synthetic polypeptide”
MAPKKKRKVGVPAAMAERPFQCRICMRNFSDRSHLSRHIRTHTGEKPFACDICGRKFADRSHLARHTKIHTGSQKPFQCRICMRNFSDRSHLTRHIRTHTGEKPFACDICGRKFALKQHLTRHTKIHTGSQKPFQCRICMRNFSWRSCRSAHIRTHTGEKPFACDICGRKFADSSHRTRHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 231 - protein, 298 aa, Artificial Sequence
  source: /note=“Description of Artificial Sequence: Synthetic polypeptide”
MAPKKKRKVGVPAAMAERPFQCRICMRNFSDRSHLSRHIRTHTGEKPFACDICGRKFADRSHLARHTKIHTGSQKPFQCRICMRNFSDRSHLTRHIRTHTGEKPFACDICGRKFALKQHLTRHTKIHTHPRAPIPKPFQCRICMRNFSRSDVLSEHIRTHTGEKPFACDICGRKFARNQHRKTHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 232 - protein, 294 aa, Artificial Sequence
  source: /note=“Description of Artificial Sequence: Synthetic polypeptide”
MAPKKKRKVGVPAAMAERPFQCRICMRNFSDRSHLSRHIRTHTGEKPFACDICGRKFADRSHLARHTKIHTGSQKPFQCRICMRNFSLRHHLTRHIRTHTGEKPFACDICGRKFAHKQHRDAHTKIHTGSQKPFQCRICMRNFSDRSHLTRHIRTHTGEKPFACDICGRKFALKQHLTRHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 233 - protein, 294 aa, Artificial Sequence
  source: /note=“Description of Artificial Sequence: Synthetic polypeptide”
MAPKKKRKVGVPAAMAERPFQCRICMRNFSDRSHLTRHIRTHTGEKPFACDICGRKFALKQHLTRHTKIHTGSQKPFQCRICMRNFSWRSCRSAHIRTHTGEKPFACDICGRKFADSSHRTRHTKIHTGSQKPFQCRICMRNFSTSGHLSRHIRTHTGEKPFACDICGRKFAQSGNLARHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 234 - protein, 294 aa, Artificial Sequence
  source: /note=“Description of Artificial Sequence: Synthetic polypeptide”
MAPKKKRKVGVPAAMAERPFQCRICMRNFSLRHHLTRHIRTHTGEKPFACDICGRKFAHKQHRDAHTKIHTGSQKPFQCRICMRNFSDRSHLTRHIRTHTGEKPFACDICGRKFALKQHLTRHTKIHTGSQKPFQCRICMRNFSQSSNLARHIRTHTGEKPFACDICGRKFAQSGHLSRHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 235 - protein, 298 aa, Artificial Sequence
  source: /note=“Description of Artificial Sequence: Synthetic polypeptide”
MAPKKKRKVGVPAAMAERPFQCRICMRNFSQSGHLARHIRTHTGEKPFACDICGRKFASPCCRYHHTKIHTGSQKPFQCRICMRNFSDRSNLSRHIRTHTGEKPFACDICGRKFATSANLSRHTKIHTHPRAPIPKPFQCRICMRNFSRSAHLSRHIRTHTGEKPFACDICGRKFADRSNLTRHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 236 - protein, 294 aa, Artificial Sequence
  source: /note=“Description of Artificial Sequence: Synthetic polypeptide”
MAPKKKRKVGVPAAMAERPFQCRICMRNFSQSGHLARHIRTHTGEKPFACDICGRKFAQLTHLNSHTKIHTGSQKPFQCRICMRNFSRSAHLSRHIRTHTGEKPFACDICGRKFADRSNLTRHTKIHTGSQKPFQCRICMRNFSRSANLARHIRTHTGEKPFACDICGRKFADRSHLARHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 237 - protein, 265 aa, Artificial Sequence
  source: /note=“Description of Artificial Sequence: Synthetic polypeptide”
MAPKKKRKVGVPAAMAERPFQCRICMRNFSRSDNLSEHIRTHTGEKPFACDICGRKFATSSNRKTHTKIHTGSQKPFQCRICMRNFSDRSNLSRHIRTHTGEKPFACDICGRKFADSSARNTHTKIHTGEKPFQCRICMRKFARNSDRTKHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 238 - protein, 298 aa, Artificial Sequence
  source: /note=“Description of Artificial Sequence: Synthetic polypeptide”
MAPKKKRKVGVPAAMAERPFQCRICMRNFSRSADLTRHIRTHTGEKPFACDICGRKFAQSANRTKHTKIHTHPRAPIPKPFQCRICMRNFSRSDNLSEHIRTHTGEKPFACDICGRKFATSSNRKTHTKIHTGSQKPFQCRICMRNFSDRSNLSRHIRTHTGEKPFACDICGRKFADSSARNTHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 239 - protein, 298 aa, Artificial Sequence
  source: /note=“Description of Artificial Sequence: Synthetic polypeptide”
MAPKKKRKVGVPAAMAERPFQCRICMRNFSQSSNLARHIRTHTGEKPFACDICGRKFADSSDRKKHTKIHTHPRAPIPKPFQCRICMRNFSRSDHLSEHIRTHTGEKPFACDICGRKFARNRDRITHTKIHTGSQKPFQCRICMRNFSRSADLTRHIRTHTGEKPFACDICGRKFAQSANRTKHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 240 - protein, 265 aa, Artificial Sequence
  source: /note=“Description of Artificial Sequence: Synthetic polypeptide”
MAPKKKRKVGVPAAMAERPFQCRICMRNFSTSGNLTRHIRTHTGEKPFACDICGRKFAQQNTLTMHTKIHTGSQKPFQCRICMRKFARSDNLTRHTKIHTGEKPFQCRICMRNFSDRSHLTRHIRTHTGEKPFACDICGRKFADRSNLTRHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 241 - protein, 294 aa, Artificial Sequence
  source: /note=“Description of Artificial Sequence: Synthetic polypeptide”
MAPKKKRKVGVPAAMAERPFQCRICMRNFSTSGNLTRHIRTHTGEKPFACDICGRKFAQQNTLTMHTKIHTGSQKPFQCRICMRNFSRSDNLARHIRTHTGEKPFACDICGRKFADRSHLARHTKIHTGSQKPFQCRICMRNFSDRSNLSRHIRTHTGEKPFACDICGRKFADAFTRTRHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 242 - protein, 302 aa, Artificial Sequence
  source: /note=“Description of Artificial Sequence: Synthetic polypeptide”
MAPKKKRKVGVPAAMAERPFQCRICMRNFSRSANLARHIRTHTGEKPFACDICGRKFADRSHLARHTKIHTHPRAPIPKPFQCRICMRNFSDRSHLTRHIRTHTGEKPFACDICGRKFADRSHLARHTKIHTHPRAPIPKPFQCRICMRNFSRSDALSEHIRTHTGEKPFACDICGRKFAQNATRTKHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 243 - protein, 294 aa, Artificial Sequence
  source: /note=“Description of Artificial Sequence: Synthetic polypeptide”
MAPKKKRKVGVPAAMAERPFQCRICMRNFSRSDSLSAHIRTHTGEKPFACDICGRKFARSDHLSRHTKIHTGSQKPFQCRICMRNFSQSGDLTRHIRTHTGEKPFACDICGRKFATQGYLRKHTKIHTGSQKPFQCRICMRNFSDRSHLSRHIRTHTGEKPFACDICGRKFADRSHLARHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 244 - protein, 298 aa, Artificial Sequence
  source: /note=“Description of Artificial Sequence: Synthetic polypeptide”
MAPKKKRKVGVPAAMAERPFQCRICMRNFSRSDHLSKHIRTHTGEKPFACDICGRKFAQNATRTKHTKIHTGSQKPFQCRICMRNFSDRSHLSRHIRTHTGEKPFACDICGRKFADRSHLARHTKIHTHPRAPIPKPFQCRICMRNFSRSAHLSRHIRTHTGEKPFACDICGRKFATSANLSRHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 245 - protein, 298 aa, Artificial Sequence
  source: /note=“Description of Artificial Sequence: Synthetic polypeptide”
MAPKKKRKVGVPAAMAERPFQCRICMRNFSQSGDLTRHIRTHTGEKPFACDICGRKFATQGYLRKHTKIHTGSQKPFQCRICMRNFSDRSHLSRHIRTHTGEKPFACDICGRKFADRSHLARHTKIHTHPRAPIPKPFQCRICMRNFSRSDNLARHIRTHTGEKPFACDICGRKFADRSHLARHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 246 - protein, 298 aa, Artificial Sequence
  source: /note=“Description of Artificial Sequence: Synthetic polypeptide”
MAPKKKRKVGVPAAMAERPFQCRICMRNFSDRSHLSRHIRTHTGEKPFACDICGRKFADRSHLARHTKIHTHPRAPIPKPFQCRICMRNFSRSAHLSRHIRTHTGEKPFACDICGRKFATSANLSRHTKIHTGSQKPFQCRICMRNFSDSSHRTRHIRTHTGEKPFACDICGRKFAMQNYLSRHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 247 - protein, 294 aa, Artificial Sequence
  source: /note=“Description of Artificial Sequence: Synthetic polypeptide”
MAPKKKRKVGVPAAMAERPFQCRICMRNFSRSDHLSNHIRTHTGEKPFACDICGRKFARSDDRKKHTKIHTGSQKPFQCRICMRNFSRSDHLSTHIRTHTGEKPFACDICGRKFATNSNRITHTKIHTGSQKPFQCRICMRNFSRSAHLSRHIRTHTGEKPFACDICGRKFADRSDLSRHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 248 - protein, 294 aa, Artificial Sequence
  source: /note=“Description of Artificial Sequence: Synthetic polypeptide”
MAPKKKRKVGVPAAMAERPFQCRICMRNFSRSDNLSNHIRTHTGEKPFACDICGRKFATSSSRINHTKIHTGSQKPFQCRICMRNFSRSDALSVHIRTHTGEKPFACDICGRKFADSSHRTRHTKIHTGSQKPFQCRICMRNFSDSSHRTRHIRTHTGEKPFACDICGRKFAAKWNLDAHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 249 - DNA, 23 bp, Homo sapiens
cggcagaaggtgggcggtggcgg

SEQ ID NO: 250 - protein, 7 aa, Artificial Sequence
  source: /note=“Description of Artificial Sequence: Synthetic peptide”
DRSHLTR

SEQ ID NO: 251 - protein, 7 aa, Artificial Sequence
  source: /note=“Description of Artificial Sequence: Synthetic peptide”
LRHHLTR

SEQ ID NO: 252 - protein, 7 aa, Artificial Sequence
  source: /note=“Description of Artificial Sequence: Synthetic peptide”
RSDDLSK

SEQ ID NO: 253 - protein, 7 aa, Artificial Sequence
  source: /note=“Description of Artificial Sequence: Synthetic peptide”
RSDNLSE

SEQ ID NO: 254 - protein, 7 aa, Artificial Sequence
  source: /note=“Description of Artificial Sequence: Synthetic peptide”
RSADLTR

SEQ ID NO: 255 - protein, 7 aa, Artificial Sequence
  source: /note=“Description of Artificial Sequence: Synthetic peptide”
RSDHLSN

SEQ ID NO: 256 - protein, 7 aa, Artificial Sequence
  source: /note=“Description of Artificial Sequence: Synthetic peptide”
RSDNLSN

SEQ ID NO: 257 - protein, 7 aa, Artificial Sequence
  source: /note=“Description of Artificial Sequence: Synthetic peptide”
QSSDLSR

SEQ ID NO: 258 - protein, 7 aa, Artificial Sequence
  source: /note=“Description of Artificial Sequence: Synthetic peptide”
RSDVLSK

SEQ ID NO: 259 - protein, 7 aa, Artificial Sequence
  source: /note=“Description of Artificial Sequence: Synthetic peptide”
DRSNLSR

SEQ ID NO: 260 - protein, 7 aa, Artificial Sequence
  source: /note=“Description of Artificial Sequence: Synthetic peptide”
RSDHLST

SEQ ID NO: 261 - protein, 7 aa, Artificial Sequence
  source: /note=“Description of Artificial Sequence: Synthetic peptide”
RSDALSV

SEQ ID NO: 262 - protein, 7 aa, Artificial Sequence
  source: /note=“Description of Artificial Sequence: Synthetic peptide”
QSGNLAR

SEQ ID NO: 263 - protein, 7 aa, Artificial Sequence
  source: /note=“Description of Artificial Sequence: Synthetic peptide”
RQDCLSL

SEQ ID NO: 264 - protein, 7 aa, Artificial Sequence
  source: /note=“Description of Artificial Sequence: Synthetic peptide”
RNSDRTK

SEQ ID NO: 265 - protein, 7 aa, Artificial Sequence
  source: /note=“Description of Artificial Sequence: Synthetic peptide”
RSAHLSR

SEQ ID NO: 266 - protein, 7 aa, Artificial Sequence
  source: /note=“Description of Artificial Sequence: Synthetic peptide”
DSSHRTR

SEQ ID NO: 267 - protein, 298 aa, Artificial Sequence
  source: /note=“Description of Artificial Sequence: Synthetic polypeptide”
MAPKKKRKVGVPAAMAERPFQCRICMQNFSDRSHLTRHIRTHTGEKPFACDICGRKFALKQHLTRHTKIHTHPRAPIPKPFQCRICMRNFSRSAHLSRHIRTHTGEKPFACDICGRKFATSGHLSRHTKIHTGSQKPFQCRICMQNFSQSGNLARHIRTHTGEKPFACDICGRKFAQSGDLTRHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 268 - protein, 294 aa, Artificial Sequence
  source: /note=“Description of Artificial Sequence: Synthetic polypeptide”
MAPKKKRKVGVPAAMAERPFQCRICMQNFSLRHHLTRHIRTHTGEKPFACDICGRKFAHRSARKRHTKIHTGSQKPFQCRICMRNFSQSSDLSRHIRTHTGEKPFACDICGRKFAHRSTRNRHTKIHTGSQKPFQCRICMRNFSDRSHLTRHIRTHTGEKPFACDICGRKFANSRNLRNHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 269 - protein, 294 aa, Artificial Sequence
  source: /note=“Description of Artificial Sequence: Synthetic polypeptide”
MAPKKKRKVGVPAAMAERPFQCRICMRNFSLRHHLTRHIRTHTGEKPFACDICGRKFAHRSARKRHTKIHTGSQKPFQCRICMQNFSQSSDLSRHIRTHTGEKPFACDICGRKFAHRSTRNRHTKIHTGSQKPFQCRICMRNFSDRSHLTRHIRTHTGEKPFACDICGRKFANSRNLRNHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 270 - protein, 294 aa, Artificial Sequence
  source: /note=“Description of Artificial Sequence: Synthetic polypeptide”
MAPKKKRKVGVPAAMAERPFQCRICMRNFSLRHHLTRHIRTHTGEKPFACDICGRKFAHRSARKRHTKIHTGSQKPFQCRICMRNFSQSSDLSRHIRTHTGEKPFACDICGRKFAHRSTRNRHTKIHTGSQKPFQCRICMQNFSDRSHLTRHIRTHTGEKPFACDICGRKFANSRNLRNHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 271 - protein, 294 aa, Artificial Sequence
  source: /note=“Description of Artificial Sequence: Synthetic polypeptide”
MAPKKKRKVGVPAAMAERPFQCRICMQNFSLRHHLTRHIRTHTGEKPFACDICGRKFAHRSARKRHTKIHTGSQKPFQCRICMQNFSQSSDLSRHIRTHTGEKPFACDICGRKFAHRSTRNRHTKIHTGSQKPFQCRICMRNFSDRSHLTRHIRTHTGEKPFACDICGRKFANSRNLRNHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 272 - protein, 294 aa, Artificial Sequence
  source: /note=“Description of Artificial Sequence: Synthetic polypeptide”
MAPKKKRKVGVPAAMAERPFQCRICMQNFSLRHHLTRHIRTHTGEKPFACDICGRKFAHRSARKRHTKIHTGSQKPFQCRICMRNFSQSSDLSRHIRTHTGEKPFACDICGRKFAHRSTRNRHTKIHTGSQKPFQCRICMQNFSDRSHLTRHIRTHTGEKPFACDICGRKFANSRNLRNHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 273 - protein, 294 aa, Artificial Sequence
  source: /note=“Description of Artificial Sequence: Synthetic polypeptide”
MAPKKKRKVGVPAAMAERPFQCRICMRNFSLRHHLTRHIRTHTGEKPFACDICGRKFAHRSARKRHTKIHTGSQKPFQCRICMQNFSQSSDLSRHIRTHTGEKPFACDICGRKFAHRSTRNRHTKIHTGSQKPFQCRICMQNFSDRSHLTRHIRTHTGEKPFACDICGRKFANSRNLRNHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 274 - protein, 294 aa, Artificial Sequence
  source: /note=“Description of Artificial Sequence: Synthetic polypeptide”
MAPKKKRKVGVPAAMAERPFQCRICMQNFSLRHHLTRHIRTHTGEKPFACDICGRKFAHRSARKRHTKIHTGSQKPFQCRICMQNFSQSSDLSRHIRTHTGEKPFACDICGRKFAHRSTRNRHTKIHTGSQKPFQCRICMQNFSDRSHLTRHIRTHTGEKPFACDICGRKFANSRNLRNHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 275 - protein, 298 aa, Artificial Sequence
  source: /note=“Description of Artificial Sequence: Synthetic polypeptide”
MAPKKKRKVGVPAAMAERPFQCRICMQNFSRSDDLSKHIRTHTGEKPFACDICGRKFAQSATRTKHTKIHTGSQKPFQCRICMRNFSRSDVLSKHIRTHTGEKPFACDICGRKFARSDDRIKHTKIHTHPRAPIPKPFQCRICMRNFSRQDCLSLHIRTHTGEKPFACDICGRKFARNDNRKTHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 276 - protein, 298 aa, Artificial Sequence
  source: /note=“Description of Artificial Sequence: Synthetic polypeptide”
MAPKKKRKVGVPAAMAERPFQCRICMRNFSRSDDLSKHIRTHTGEKPFACDICGRKFAQSATRTKHTKIHTGSQKPFQCRICMQNFSRSDVLSKHIRTHTGEKPFACDICGRKFARSDDRIKHTKIHTHPRAPIPKPFQCRICMRNFSRQDCLSLHIRTHTGEKPFACDICGRKFARNDNRKTHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 277 - protein, 298 aa, Artificial Sequence
  source: /note=“Description of Artificial Sequence: Synthetic polypeptide”
MAPKKKRKVGVPAAMAERPFQCRICMRNFSRSDDLSKHIRTHTGEKPFACDICGRKFAQSATRTKHTKIHTGSQKPFQCRICMRNFSRSDVLSKHIRTHTGEKPFACDICGRKFARSDDRIKHTKIHTHPRAPIPKPFQCRICMQNFSRQDCLSLHIRTHTGEKPFACDICGRKFARNDNRKTHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 278 - protein, 298 aa, Artificial Sequence
  source: /note=“Description of Artificial Sequence: Synthetic polypeptide”
MAPKKKRKVGVPAAMAERPFQCRICMQNFSRSDDLSKHIRTHTGEKPFACDICGRKFAQSATRTKHTKIHTGSQKPFQCRICMQNFSRSDVLSKHIRTHTGEKPFACDICGRKFARSDDRIKHTKIHTHPRAPIPKPFQCRICMRNFSRQDCLSLHIRTHTGEKPFACDICGRKFARNDNRKTHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 279 - protein, 298 aa, Artificial Sequence
  source: /note=“Description of Artificial Sequence: Synthetic polypeptide”
MAPKKKRKVGVPAAMAERPFQCRICMRNFSRSDDLSKHIRTHTGEKPFACDICGRKFAQSATRTKHTKIHTGSQKPFQCRICMQNFSRSDVLSKHIRTHTGEKPFACDICGRKFARSDDRIKHTKIHTHPRAPIPKPFQCRICMQNFSRQDCLSLHIRTHTGEKPFACDICGRKFARNDNRKTHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 280 - protein, 298 aa, Artificial Sequence
  source: /note=“Description of Artificial Sequence: Synthetic polypeptide”
MAPKKKRKVGVPAAMAERPFQCRICMQNFSRSDDLSKHIRTHTGEKPFACDICGRKFAQSATRTKHTKIHTGSQKPFQCRICMQNFSRSDVLSKHIRTHTGEKPFACDICGRKFARSDDRIKHTKIHTHPRAPIPKPFQCRICMQNFSRQDCLSLHIRTHTGEKPFACDICGRKFARNDNRKTHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 281 - protein, 265 aa, Artificial Sequence
  source: /note=“Description of Artificial Sequence: Synthetic polypeptide”
MAPKKKRKVGVPAAMAERPFQCRICMQNFSRSDNLSEHIRTHTGEKPFACDICGRKFATSSNRKTHTKIHTGSQKPFQCRICMRNFSDRSNLSRHIRTHTGEKPFACDICGRKFADSSARNTHTKIHTGEKPFQCRICMRKFARNSDRTKHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 282 - protein, 265 aa, Artificial Sequence
  source: /note=“Description of Artificial Sequence: Synthetic polypeptide”
MAPKKKRKVGVPAAMAERPFQCRICMRNFSRSDNLSEHIRTHTGEKPFACDICGRKFATSSNRKTHTKIHTGSQKPFQCRICMRNFSDRSNLSRHIRTHTGEKPFACDICGRKFADSSARNTHTKIHTGEKPFQCRICMQKFARNSDRTKHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 283 - protein, 265 aa, Artificial Sequence
  source: /note=“Description of Artificial Sequence: Synthetic polypeptide”
MAPKKKRKVGVPAAMAERPFQCRICMQNFSRSDNLSEHIRTHTGEKPFACDICGRKFATSSNRKTHTKIHTGSQKPFQCRICMQNFSDRSNLSRHIRTHTGEKPFACDICGRKFADSSARNTHTKIHTGEKPFQCRICMRKFARNSDRTKHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 284 - protein, 265 aa, Artificial Sequence
  source: /note=“Description of Artificial Sequence: Synthetic polypeptide”
MAPKKKRKVGVPAAMAERPFQCRICMQNFSRSDNLSEHIRTHTGEKPFACDICGRKFATSSNRKTHTKIHTGSQKPFQCRICMRNFSDRSNLSRHIRTHTGEKPFACDICGRKFADSSARNTHTKIHTGEKPFQCRICMQKFARNSDRTKHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 285 - protein, 265 aa, Artificial Sequence
  source: /note=“Description of Artificial Sequence: Synthetic polypeptide”
MAPKKKRKVGVPAAMAERPFQCRICMRNFSRSDNLSEHIRTHTGEKPFACDICGRKFATSSNRKTHTKIHTGSQKPFQCRICMQNFSDRSNLSRHIRTHTGEKPFACDICGRKFADSSARNTHTKIHTGEKPFQCRICMQKFARNSDRTKHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 286 - protein, 265 aa, Artificial Sequence
  source: /note=“Description of Artificial Sequence: Synthetic polypeptide”
MAPKKKRKVGVPAAMAERPFQCRICMQNFSRSDNLSEHIRTHTGEKPFACDICGRKFATSSNRKTHTKIHTGSQKPFQCRICMQNFSDRSNLSRHIRTHTGEKPFACDICGRKFADSSARNTHTKIHTGEKPFQCRICMQKFARNSDRTKHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 287 - protein, 298 aa, Artificial Sequence
  source: /note=“Description of Artificial Sequence: Synthetic polypeptide”
MAPKKKRKVGVPAAMAERPFQCRICMQNFSRSADLTRHIRTHTGEKPFACDICGRKFAQSANRTKHTKIHTHPRAPIPKPFQCRICMRNFSRSDNLSEHIRTHTGEKPFACDICGRKFATSSNRKTHTKIHTGSQKPFQCRICMRNFSDRSNLSRHIRTHTGEKPFACDICGRKFADSSARNTHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 288 - protein, 298 aa, Artificial Sequence
  source: /note=“Description of Artificial Sequence: Synthetic polypeptide”
MAPKKKRKVGVPAAMAERPFQCRICMRNFSRSADLTRHIRTHTGEKPFACDICGRKFAQSANRTKHTKIHTHPRAPIPKPFQCRICMQNFSRSDNLSEHIRTHTGEKPFACDICGRKFATSSNRKTHTKIHTGSQKPFQCRICMRNFSDRSNLSRHIRTHTGEKPFACDICGRKFADSSARNTHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 289 - protein, 298 aa, Artificial Sequence
  source: /note=“Description of Artificial Sequence: Synthetic polypeptide”
MAPKKKRKVGVPAAMAERPFQCRICMRNFSRSADLTRHIRTHTGEKPFACDICGRKFAQSANRTKHTKIHTHPRAPIPKPFQCRICMRNFSRSDNLSEHIRTHTGEKPFACDICGRKFATSSNRKTHTKIHTGSQKPFQCRICMQNFSDRSNLSRHIRTHTGEKPFACDICGRKFADSSARNTHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 290 - protein, 298 aa, Artificial Sequence
  source: /note=“Description of Artificial Sequence: Synthetic polypeptide”
MAPKKKRKVGVPAAMAERPFQCRICMQNFSRSADLTRHIRTHTGEKPFACDICGRKFAQSANRTKHTKIHTHPRAPIPKPFQCRICMQNFSRSDNLSEHIRTHTGEKPFACDICGRKFATSSNRKTHTKIHTGSQKPFQCRICMRNFSDRSNLSRHIRTHTGEKPFACDICGRKFADSSARNTHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 291 - protein, 298 aa, Artificial Sequence
  source: /note=“Description of Artificial Sequence: Synthetic polypeptide”
MAPKKKRKVGVPAAMAERPFQCRICMQNFSRSADLTRHIRTHTGEKPFACDICGRKFAQSANRTKHTKIHTHPRAPIPKPFQCRICMRNFSRSDNLSEHIRTHTGEKPFACDICGRKFATSSNRKTHTKIHTGSQKPFQCRICMQNFSDRSNLSRHIRTHTGEKPFACDICGRKFADSSARNTHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 292 - protein, 298 aa, Artificial Sequence
  source: /note=“Description of Artificial Sequence: Synthetic polypeptide”
MAPKKKRKVGVPAAMAERPFQCRICMQNFSRSADLTRHIRTHTGEKPFACDICGRKFAQSANRTKHTKIHTHPRAPIPKPFQCRICMQNFSRSDNLSEHIRTHTGEKPFACDICGRKFATSSNRKTHTKIHTGSQKPFQCRICMQNFSDRSNLSRHIRTHTGEKPFACDICGRKFADSSARNTHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 293 - protein, 294 aa, Artificial Sequence
  source: /note=“Description of Artificial Sequence: Synthetic polypeptide”
MAPKKKRKVGVPAAMAERPFQCRICMQNFSRSDHLSNHIRTHTGEKPFACDICGRKFARSDDRKKHTKIHTGSQKPFQCRICMRNFSRSDHLSTHIRTHTGEKPFACDICGRKFATNSNRITHTKIHTGSQKPFQCRICMRNFSRSAHLSRHIRTHTGEKPFACDICGRKFADRSDLSRHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 294 - protein, 294 aa, Artificial Sequence
  source: /note=“Description of Artificial Sequence: Synthetic polypeptide”
MAPKKKRKVGVPAAMAERPFQCRICMRNFSRSDHLSNHIRTHTGEKPFACDICGRKFARSDDRKKHTKIHTGSQKPFQCRICMQNFSRSDHLSTHIRTHTGEKPFACDICGRKFATNSNRITHTKIHTGSQKPFQCRICMRNFSRSAHLSRHIRTHTGEKPFACDICGRKFADRSDLSRHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 295 - protein, 294 aa, Artificial Sequence
  source: /note=“Description of Artificial Sequence: Synthetic polypeptide”
MAPKKKRKVGVPAAMAERPFQCRICMRNFSRSDHLSNHIRTHTGEKPFACDICGRKFARSDDRKKHTKIHTGSQKPFQCRICMRNFSRSDHLSTHIRTHTGEKPFACDICGRKFATNSNRITHTKIHTGSQKPFQCRICMQNFSRSAHLSRHIRTHTGEKPFACDICGRKFADRSDLSRHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 296 - protein, 294 aa, Artificial Sequence
  source: /note=“Description of Artificial Sequence: Synthetic polypeptide”
MAPKKKRKVGVPAAMAERPFQCRICMQNFSRSDHLSNHIRTHTGEKPFACDICGRKFARSDDRKKHTKIHTGSQKPFQCRICMQNFSRSDHLSTHIRTHTGEKPFACDICGRKFATNSNRITHTKIHTGSQKPFQCRICMRNFSRSAHLSRHIRTHTGEKPFACDICGRKFADRSDLSRHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 297 - protein, 294 aa, Artificial Sequence
  source: /note=“Description of Artificial Sequence: Synthetic polypeptide”
MAPKKKRKVGVPAAMAERPFQCRICMQNFSRSDHLSNHIRTHTGEKPFACDICGRKFARSDDRKKHTKIHTGSQKPFQCRICMRNFSRSDHLSTHIRTHTGEKPFACDICGRKFATNSNRITHTKIHTGSQKPFQCRICMQNFSRSAHLSRHIRTHTGEKPFACDICGRKFADRSDLSRHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 298 - protein, 294 aa, Artificial Sequence
  source: /note=“Description of Artificial Sequence: Synthetic polypeptide”
MAPKKKRKVGVPAAMAERPFQCRICMRNFSRSDHLSNHIRTHTGEKPFACDICGRKFARSDDRKKHTKIHTGSQKPFQCRICMQNFSRSDHLSTHIRTHTGEKPFACDICGRKFATNSNRITHTKIHTGSQKPFQCRICMQNFSRSAHLSRHIRTHTGEKPFACDICGRKFADRSDLSRHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 299 - protein, 294 aa, Artificial Sequence
  source: /note=“Description of Artificial Sequence: Synthetic polypeptide”
MAPKKKRKVGVPAAMAERPFQCRICMQNFSRSDHLSNHIRTHTGEKPFACDICGRKFARSDDRKKHTKIHTGSQKPFQCRICMQNFSRSDHLSTHIRTHTGEKPFACDICGRKFATNSNRITHTKIHTGSQKPFQCRICMQNFSRSAHLSRHIRTHTGEKPFACDICGRKFADRSDLSRHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 300 - protein, 294 aa, Artificial Sequence
  source: /note=“Description of Artificial Sequence: Synthetic polypeptide”
MAPKKKRKVGVPAAMAERPFQCRICMQNFSRSDNLSNHIRTHTGEKPFACDICGRKFATSSSRINHTKIHTGSQKPFQCRICMRNFSRSDALSVHIRTHTGEKPFACDICGRKFADSSHRTRHTKIHTGSQKPFQCRICMRNFSDSSHRTRHIRTHTGEKPFACDICGRKFAAKWNLDAHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 301 - protein, 294 aa, Artificial Sequence
  source: /note=“Description of Artificial Sequence: Synthetic polypeptide”
MAPKKKRKVGVPAAMAERPFQCRICMRNFSRSDNLSNHIRTHTGEKPFACDICGRKFATSSSRINHTKIHTGSQKPFQCRICMQNFSRSDALSVHIRTHTGEKPFACDICGRKFADSSHRTRHTKIHTGSQKPFQCRICMRNFSDSSHRTRHIRTHTGEKPFACDICGRKFAAKWNLDAHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 302 - protein, 294 aa, Artificial Sequence
  source: /note=“Description of Artificial Sequence: Synthetic polypeptide”
MAPKKKRKVGVPAAMAERPFQCRICMRNFSRSDNLSNHIRTHTGEKPFACDICGRKFATSSSRINHTKIHTGSQKPFQCRICMRNFSRSDALSVHIRTHTGEKPFACDICGRKFADSSHRTRHTKIHTGSQKPFQCRICMQNFSDSSHRTRHIRTHTGEKPFACDICGRKFAAKWNLDAHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 303 - protein, 294 aa, Artificial Sequence
  source: /note=“Description of Artificial Sequence: Synthetic polypeptide”
MAPKKKRKVGVPAAMAERPFQCRICMQNFSRSDNLSNHIRTHTGEKPFACDICGRKFATSSSRINHTKIHTGSQKPFQCRICMQNFSRSDALSVHIRTHTGEKPFACDICGRKFADSSHRTRHTKIHTGSQKPFQCRICMRNFSDSSHRTRHIRTHTGEKPFACDICGRKFAAKWNLDAHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 304 - protein, 294 aa, Artificial Sequence
  source: /note=“Description of Artificial Sequence: Synthetic polypeptide”
MAPKKKRKVGVPAAMAERPFQCRICMQNFSRSDNLSNHIRTHTGEKPFACDICGRKFATSSSRINHTKIHTGSQKPFQCRICMRNFSRSDALSVHIRTHTGEKPFACDICGRKFADSSHRTRHTKIHTGSQKPFQCRICMQNFSDSSHRTRHIRTHTGEKPFACDICGRKFAAKWNLDAHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 305 - protein, 294 aa, Artificial Sequence
  source: /note=“Description of Artificial Sequence: Synthetic polypeptide”
MAPKKKRKVGVPAAMAERPFQCRICMRNFSRSDNLSNHIRTHTGEKPFACDICGRKFATSSSRINHTKIHTGSQKPFQCRICMQNFSRSDALSVHIRTHTGEKPFACDICGRKFADSSHRTRHTKIHTGSQKPFQCRICMQNFSDSSHRTRHIRTHTGEKPFACDICGRKFAAKWNLDAHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 306 - protein, 294 aa, Artificial Sequence
  source: /note=“Description of Artificial Sequence: Synthetic polypeptide”
MAPKKKRKVGVPAAMAERPFQCRICMQNFSRSDNLSNHIRTHTGEKPFACDICGRKFATSSSRINHTKIHTGSQKPFQCRICMQNFSRSDALSVHIRTHTGEKPFACDICGRKFADSSHRTRHTKIHTGSQKPFQCRICMQNFSDSSHRTRHIRTHTGEKPFACDICGRKFAAKWNLDAHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 307 - protein, 616 aa, Artificial Sequence
  source: /note=“Description of Artificial Sequence: Synthetic polypeptide”
MAPKKKRKVGVPAAMAERPFQCRICMQNFSTDRSHLRHIRTHTGEKPFACDICGRKFALKQHLTRHTKIHTHPRAPIPKPFQCRICMRNFSRSAHLSRHIRTHTGEKPFACDICGRKFATSGHLSRHTKIHTGSQKPFQCRICMQNFSQSGNLARHIRTHTGEKPFACDICGRKFAQSGDLTRHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRSGSGEGRGSLLTCGDVEENPGPRATMAPKKKRKVGVPAAMAERPFQCRICMRNFSLRHHLTRHIRTHTGEKPFACDICGRKFARRFTLSKHTKIHTGSQKPFQCRICMRNFSRSDVLSEHIRTHTGEKPFACDICGRKFAKHSTRRVHTKIHTGSQKPFQCRICMRNFSRSDVLSEHIRTHTGEKPFACDICGRKFARLYTLHKHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVKL

SEQ ID NO: 308 - DNA, 18 bp, Homo sapiens
tacccaacgcgaattgcg

SEQ ID NO: 309 - DNA, 285 bp, Mus sp.
tctccgccacccaccagctccagcaccagcagcagcgccgccgccaccgcccaccttctgccgccgccgccacaaccaccttctcctccgctgtcctcttctgtcctcgccttctgtcgattatcaggtaagcgccgcagctctgaaatccagtcccctctggccctgtgcgccgtcctcctgccacccctcgccccagagatggatttagggagctgggcatcaatcaggcgcaacggcgggggcctctctgcgccctaggcgctggggagctgggaaggaacg

SEQ ID NO: 310 - DNA, 313 bp, Unknown
  source: /note=“Description of Unknown: NHP(mf) sequence”
tcaccgccacccaccagctccggcaccaacagcagcgccgctgccaccgcccaccttctgccgccgccaccacagccaccttctcctcctccgctgtcctctcccgtcctcgcctctgtcgactatcaggtaagcgccgcggctccgaaatctgcctcgccgtccgcccctgtgcacccctgcgccgccgcccctcgccctcagcctccacagactggggcttcgtgcgccgggcatcggtcggggcgaccgcagggcccctccttgcctcccctgctcgggggctggggccagggcggcctggaaagggacg

SEQ ID NO: 311 - DNA, 313 bp, Homo sapiens
tcaccgccacccaccagctccggcaccaacagcagcgccgctgccaccgcccaccttctgccgccgccaccacagccaccttctcctcctccgctgtcctctcccgtcctcgcctctgtcgactatcaggtaagcgccgcggctccgaaatctgcctcgccgtccgcctctgtgcacccctgcgccgccgcccctcgccctccctctccgcagactggggcttcgtgcgccgggcatcggtcggggccaccgcagggcccctccctgcctcccctgctcgggggctggggccagggcggcctggaaagggacc

(19) United States
(12) Patent Application Publication
(10) Pub. No.: US 2023/0242602 A1
(43) Pub. Date: Aug. 3, 2023

(54) ZINC FINGER PROTEIN TRANSCRIPTION FACTORS FOR REPRESSING TAU EXPRESSION

(71) Applicant: Sangamo Therapeutics, Inc., Brisbane, CA (US)

(72) Inventors: Bryan Zeitler, El Sobrante, CA (US); Asa Hatami, Pleasanton, CA (US); Lei Zhang, Davis, CA (US)

(73) Assignee: Sangamo Therapeutics, Inc., Brisbane, CA (US)

(21) Appl. No.: 17/791,395

(22) PCT Filed: Jan. 22, 2021
(86) PCT No.: PCT/US2021/014780

§ 371 Date: Jul. 7, 2022

Related U.S. Application Data
(60) Provisional application No. 62/964,501, filed on Jan. 22, 2020.

(51) Int. Cl. C07K 14/47; C12N 15/86; A61P 25/28

(52) U.S. Cl. CPC C07K 14/4703; C07K 14/4711; C12N 15/86; A61P 25/28; C12N 2750/14143; C07K 2319/71

(57) ABSTRACT

The present disclosure provides zinc finger fusion proteins that inhibit expression of tau in the nervous system, and methods of using the proteins to treat neurodegenerative diseases such as Alzheimer's disease, frontotemporal dementia, and other tauopathies.

### CROSS REFERENCE TO RELATED APPLICATIONS

The present application claims priority from U.S. Provisional Application 62/964,501, filed on Jan. 22, 2020. The contents of the aforementioned provisional application are incorporated herein by reference in their entirety.

### SEQUENCE LISTING

The instant application contains a Sequence Listing which has been submitted electronically in ASCII format and is hereby incorporated by reference in its entirety. Said ASCII copy, created on Jan. 19, 2021, is named 025297_WO016_SL.txt and is 304,337 bytes in size.

### BACKGROUND OF THE INVENTION

Microtubule-associated protein tau (MAPT), also known as tau, plays an important role in certain brain pathologies. The aggregation of misfolded tau into neurofibrillary tangles (NFTs) and other pathological tau inclusions is implicated in a number of neurodegenerative conditions collectively referred to as tauopathies. These include Alzheimer's disease (AD), frontotemporal dementia (FTD), progressive supranuclear palsy (PSP), intractable genetic epilepsies (e.g., Dravet syndrome), traumatic brain injury (TBI), corticobasal degeneration (CBD), and chronic traumatic encephalopathy (CTE). See, e.g., Benussi et al., Front Aging Neurosci. (2015) 7:171; Gheyara et al., Ann Neurol. (2014) 76:443-56; Scholz and Bras, Int J Mol Sci. (2015) 16(10):24629-55; and McKee et al., Brain Pathol. (2015) 25(3):350-64.

It has been suggested that tau has prion-like properties. Several studies show that hyperphosphorylation of tau can cause tau misfolding. Misfolded tau aggregates can spread throughout the brain (Takeda et al., Nat Comm. (2015) 6:8490; Hyman, Neuron (2014) 82:1189; de Calignon et al., Neuron (2012) 73:685-97). These aggregates may be the initial step in the formation of NFTs found in tauopathies. Although NFTs are restricted to the entorhinal cortex and medial temporal lobe in the early stages of AD, by the time severe clinical symptoms appear, NFTs are widespread throughout the brain. Both NFTs and amyloid-beta plaques are found in patients with AD, and amyloid deposition has been shown to increase tau pathology and deposition in distal brain regions (Bennett et al., Am J Pathol. (2017) 187(7):1601-12). Regional tau accumulation and spreading is closely linked to neuronal loss in rodent models and human disease (Pooler et al., Acta Neuropathol Commun. (2015) 3:14; La Joie et al., Sci Transl Med. (2020) 12:524). In addition to the neurotoxicity exerted by the accumulation of aggregated tau, soluble oligomeric forms of tau appear to be toxic as well (Guerrero-Munoz et al., Front Cell Neurosci. (2015) 9:464). Soluble misfolded endogenous tau seems to play a role as a mediator of neurotoxicity in various neuronal stress conditions.

Reduction of endogenous tau has been shown to be beneficial for AD-like pathology in different genetic mouse models (Roberson et al., Science (2007) 316:750-4; DeVos et al., Sci Transl Med. (2017) 9(374):eaag0481; Wegmann et al., EMBO J. (2015) 1-14). Decreasing tau levels in the brain also appears to protect against stress-induced and seizure-induced neuronal damage, and against learning and memory deficits resulting from traumatic brain injury (Lopes et al., PNAS (2016) 113:e3755-63; Gheyara, supra; DeVos et al., J Neurosci. (2013) 33(31):12887-97; Cheng et al., PLoS One (2014) 9(12):e115765). However, there has been no effective treatment for tauopathies. Tau knock-down in vivo has been achieved through administration of antisense oligonucleotides (ASOs) that bind tau mRNA and prevent its translation (DeVos (2017) ibid; DeVos et al., Neurotherapeutics (2013) 10(3):486-97) or by intravenous injections of anti-tau antibodies (Asuni et al., J Neurosci. (2007) 27:9115-29; Ittner et al., J Neurochem. (2015) 132:135-45; Herrmann et al., J Neurochem. (2015) 132:1-4; Yanamandra et al., Neuron (2013) 80(2):402-14). Although both approaches may facilitate tau protein reduction in the brain, they require chronic administrations for the lifetime of the patient. Antibodies have poor blood-brain barrier and cell membrane permeability, which can limit both their spread within the central nervous system and their ability to engage intraneuronal tau. Moreover, the development of anti-tau antibody therapeutics has been difficult because the identity and number of pathogenic tau species is currently unknown and may vary among tauopathies.

Given the significant role of tau in brain pathologies and the lack of effective treatment, there is an urgent need to develop therapeutics targeting this protein for the prevention and treatment of tauopathies, including AD.

### SUMMARY OF THE INVENTION

The present disclosure provides zinc finger proteins (ZFPs) that target sites in or near the human MAPT gene. The ZFPs of the present disclosure may be fused to a transcription factor to specifically inhibit expression of a microtubule-associated protein tau (MAPT) gene at the DNA level. These fusion proteins contain (i) a ZFP domain that binds specifically to a target region in the MAPT gene and (ii) at least one transcription repressor domain that reduces the transcription of the gene.

In one aspect, the present disclosure provides a fusion protein comprising a zinc finger protein (ZFP) domain and a transcription repressor domain, wherein the ZFP domain binds to a target region of a human MAPT gene. In some embodiments, the target region is within 1.5 kb of a transcription start site (TSS) in the MAPT gene, for example, within 1000 bps upstream of the TSS, and/or within 500 bps downstream of the TSS of the MAPT gene.

In some embodiments, the ZFP domain comprises one or more (e.g., one, two, three, four, five, six, or more) zinc fingers and the fusion protein optionally represses expression of the MAPT gene by at least about 40%, 75%, 90%, 95%, or 99%, optionally with no or minimal off-target binding or activity (e.g., binding to a gene that is not the MAPT gene) detectable by a well-known method. Nonlimiting examples of DNA-binding recognition helix amino acid sequences of the present ZFPs are shown in FIG. 14 or FIG. 16. In some embodiments, the fusion protein comprises one or more recognition helix sequence shown in FIG. 14 or FIG. 16. In further embodiments, the fusion protein comprises some or all the recognition helix sequences shown in a single row in FIG. 14 or FIG. 16, with or without the indicated backbone mutation(s) shown in FIG. 16. In certain embodiments, the fusion protein comprises an amino acid sequence shown in FIG. 15 or FIG. 17.

In some embodiments, the transcription repressor domain of the present fusion protein comprises a KRAB domain, wherein the KRAB domain optionally is from a human KOX1 protein. In some embodiments, the ZFP domain is linked to the transcription repressor through a peptide linker. In some embodiments, the fusion protein comprises a nuclear localization signal.

In some embodiments, the ZFP domain of the fusion protein comprises four, five, or six zinc finger recognition helix sequences shown in a single row of FIG. 14 or FIG. 16; binds to a target sequence shown in FIG. 14 or FIG. 16; comprises the zinc finger recognition helix sequences of a ZFP transcription factor shown in FIG. 15 or FIG. 17 (e.g., ZFP-TFs 71377, 71385, 73034, 73122, 73131, or 73133); and/or comprises the zinc finger recognition helix sequences linked as shown in FIG. 14, FIG. 15, FIG. 16, or FIG. 17 (e.g., ZFP-TFs 71377, 71385, 73034, 73122, 73131, or 73133).

In particular embodiments, the present fusion protein comprises an amino acid sequence selected from SEQ ID NOs: 89-196, 197-248 and 267-307.

In another aspect, the present disclosure provides a nucleic acid construct comprising a coding sequence for the present fusion protein, wherein the coding sequence is linked operably to a transcription regulatory element. In some embodiments, the transcription regulatory element is a mammalian promoter that is constitutively active or inducible in a brain cell. In certain embodiments, the construct is a recombinant viral construct such as a recombinant adeno-associated viral (AAV) construct.

In another aspect, the present disclosure provides a host cell comprising the present nucleic acid construct. The host cell may be a human cell, such as a human brain cell or pluripotent stem cell (e.g., an embryonic stem cell or an inducible pluripotent stem cell (iPSC)).

In yet another aspect, the present disclosure provides a method of inhibiting expression of tau in a human brain cell (e.g., a neuron, a glial cell, an ependymal cell, a neuroepithelial cell, an endothelial cell, or an oligodendrocyte), comprising introducing into the cell a fusion protein herein, optionally through introduction of a nucleic acid construct herein, thereby inhibiting the expression of tau in the cell. In some embodiments, the cell is in the brain of a patient suffering from or at risk of developing Alzheimer's disease, frontotemporal dementia, progressive supranuclear palsy, traumatic brain injury (TBI), seizure disorders, corticobasal degeneration (CBD), chronic traumatic encephalopathy (CTE), or another tauopathy. In particular embodiments, the method comprises introducing into the cell a recombinant virus that expresses the fusion protein (e.g., a recombinant AAV of a neurotrophic serotype or pseudotype such as AAV9 and the like).

In a related aspect, the present disclosure provides a method of treating (e.g., slowing the progression of) a tauopathy in a patient, comprising administering to the patient a recombinant AAV encoding a fusion protein herein. In some embodiments, the AAV is introduced to the patient via an intravenous, intrathecal, intracerebral, intracerebroventricular, intra-cisternal magna, intrahippocampal, intrathalamic, or intraparenchymal route. In some embodiments, the tauopathy is Alzheimer's disease, or frontotemporal dementia, progressive supranuclear palsy, traumatic brain injury (TBI), seizure disorders, corticobasal degeneration (CBD), or chronic traumatic encephalopathy (CTE).

Also provided herein are fusion proteins for use in a treatment method described herein, and the use of a fusion protein herein for the manufacture of a medicament in the treatment method.

Other features, objectives, and advantages of the invention are apparent in the detailed description that follows. It should be understood, however, that the detailed description, while indicating embodiments and aspects of the invention, is given by way of illustration only, not limitation. Various changes and modification within the scope of the invention will become apparent to those skilled in the art from the detailed description.

### BRIEF DESCRIPTION OF THE DRAWINGS

FIG. 1 is a diagram illustrating specific targeting of the MAPT gene by an engineered 6-finger zinc finger protein-transcription factor (ZFP-TF), which recognizes 18 base pairs in the gene. Binding of the ZFP-TF to the gene leads to reduced MAPT transcription, which in turn leads to reduced MAPT mRNA and tau protein levels. The figure discloses SEQ ID NO: 308.

FIG. 2 a diagram illustrating the targeting of the MAPT gene by anti-tau ZFP-TFs. The primary MAPT mRNA is shown in the top bar, with exon 1 represented by a thick arrow. The small pentagons (370; orange and blue) in the clusters underneath the gene and the mRNA depict the regions in the MAPT gene targeted by representative ZFP-TFs exemplified herein. The blue pentagons depict the ZFP-TFs that specifically target the human MAPT gene whereas the orange pentagons target both human and non-human primate (NHP) MAPT genes. Pentagons pointing to the right indicate that the ZFP-TFs bind the sense strand of the gene. Pentagons pointing to the left indicate that the ZFP-TFs bind the antisense strand of the gene.

FIG. 3 is a panel of graphs showing the tau-repressing activity of 48 ZFP-TFs selected from a library of 370 ZFP-TFs (FIG. 14). The y-axis in each graph is tau mRNA expression normalized to the geometric mean of two housekeeping genes (Atp5b and Eif4a2) and assessed 20 hours after transfection with mRNA coding for the different ZFP-TFs in SK-N-MC cells. The mRNA dose increases from left to right (3, 10, 30, 100, 300, and 1,000 ng). The bars represent the mean of four technical replicates and the error bars represent standard deviation. The numbers below the graphs are the internal reference numbers for the ZFP-TFs. An enlarged version of the titration scale is shown at the lower right of the figure.

FIG. 4 is a diagram illustrating an optimization strategy for improving the target specificity of the parent ZFP-TFs. It involves mutating an arginine (R) residue to a glutamine (Q) at positions (−4), (−5), (−9) and/or (−14) in up to three zinc finger modules of the parent ZFP-TFs. The mutation impacts a conserved non-specific contact between the zinc finger and the phosphate backbone of the target DNA.

FIGS. 5A and 5B are diagrams illustrating the target regions of the mutant anti-tau ZFP-TFs in the MAPT gene. The MAPT mRNA is shown as a red bar. In FIG. 5A, the small pentagons (red and white) in the first cluster underneath the gene depict the regions in the MAPT gene targeted by the parent ZFP-TFs. The small pentagons (red) in the second cluster underneath the gene depict the regions in the MAPT gene targeted by the R→Q variants of the parent ZFP-TFs. FIG. 5B shows an enlarged view of the target regions of representative parent and mutant ZFP-TFs in the MAPT gene. The orange pentagons represent the parent ZFP-TFs and the red pentagons represent their R→Q variants. Pentagons pointing to the right indicate that the ZFP-TFs bind the sense strand of the gene. Pentagons pointing to the left indicate that the ZFP-TFs bind the antisense strand of the gene. Parental ZFP-TFs are numbered with 71 ### while mutant ZFP-TFs are numbered with 73 ###. FIG. 5B discloses SEQ ID NOS: 309-311, respectively, in order of appearance.

FIG. 6 is a panel of graphs showing the tau-repressing activity of representative R→Q variant ZFP-TFs (FIG. 16) from a library of ˜340 variants. The y-axis in each graph is tau mRNA expression normalized to the geometric mean of two housekeeping genes (Atp5b and Eif4a2) and assessed 20 hours after transfection with mRNA coding for the different ZFP-TFs in SK-N-MC cells. The mRNA dose increases from left to right (3, 10, 30, 100, 300, and 1,000 ng). Up to seven R→Q variants are shown for each parent ZFP-TF. The bars represent the mean of four technical replicates and the error bars represent standard deviation. The numbers below the graphs are the internal reference numbers for the ZFP-TFs. An enlarged version of the titration scale is shown at the lower right of the figure.

FIG. 7A is a panel of graphs showing the tau-repressing activity of representative ZFP-TFs in human iPSC-derived neurons. The y-axis is tau mRNA expression normalized to the geometric mean of three housekeeping genes (ATP5B, EIF4A2 and GAPDH) and assessed 32 days after transduction with AAV6 for the different ZFP-TFs in human iPSC-derived neurons. The amount of AAV6 used is indicated in the legend on the lower right, with the AAV6 dose increasing from left to right (MOI of 1E3, 3E3, 1E4, 3E4, 1E5, and 3E5). Neurons treated with formulation buffer (Mock) were used as a negative control. The bars represent the mean of four technical replicates and the error bars represent standard deviation.

FIG. 7B is a panel of graphs comparing the human tau-repressing activity of representative R→Q variant ZFP-TFs in human iPSC-derived neurons, human SK-N-MC cells, and primary mouse neurons. The y-axis is tau mRNA expression normalized to the geometric mean of the housekeeping genes ATP5B, EIF4A2 and optionally GAPDH, and assessed 32 days and 7 days after transduction with AAV6 for the different ZFP-TFs in human iPSC-derived neurons and primary mouse neurons, respectively; or assessed 20 hours after transfection with mRNA coding for the different ZFP-TFs in SK-N-MC cells. The amount of AAV6 or mRNA used is indicated under the x-axis, with the AAV6 (MOI of 1E3, 3E3, 1E4, 3E4, 1E5, and 3E5) and mRNA (3, 10, 30, 100, 300, and 1,000 ng) doses increasing from left to right. The bars represent the mean of four technical replicates and the error bars represent standard deviation. Enlarged versions of the titration scales are shown below the figure. The number of mismatches between the targeted sequence in human MAPT and orthologous target site in mouse Mapt is indicated above the bars for each ZFP tested in primary mouse neurons.

FIG. 8 is a panel of volcano/scatter plots of Affymetrix/microarray data showing changes in the transcriptomes of human iPSC-derived neurons and primary mouse neurons after 19 days or 7 days, respectively, following treatment with representative R→Q variant ZFP-TFs. The blue rectangles indicate the level of human tau repression achieved with each representative ZFP-TF at the highest dose tested in neurons. Numbers shown in red and green indicate the counts of downregulated and upregulated off-target genes, respectively. Yellow circles represent different transcripts within the tau locus; red circles represent downregulated off-target genes; and green circles represent upregulated off-target genes. Human and mouse microarray data were derived from at least two independent experiments and 5-8 biological replicates per experiment. CPNE6 was shown to be an artifact of the reference ZFP and therefore excluded from the off-target counts.

FIG. 9 is a panel of graphs showing the effects of representative R→Q variant ZFP-TFs on the expression levels of transcripts within the tau locus (human tau and STH) and off-target genes (CPNE6 and IGF2). Mock treatments and a ZFP-TF that does not bind to the tau locus were used as negative controls. The y-axis is mRNA expression normalized to the geometric mean of three housekeeping genes (ATP5B, EIF4A2, and GAPDH) and assessed 32 days after transfection with mRNA coding for the different ZFP-TFs in human iPSC-derived neurons. The amount of AAV6 used is indicated in the legend on the lower right, with the AAV6 dose increasing from left to right (MOI of 1E3, 3E3, 1E4, 3E4, 1E5, and 3E5). The blue rectangles indicate the level of human tau repression achieved with each representative ZFP-TF at the maximum dose tested in neurons. The bars represent the mean of four technical replicates and the error bars represent standard deviation.

FIG. 10 is a panel of graphs showing ZFP-TF, mouse Mapt, NeuN, and neuroinflammatory marker mRNA expression levels following intraparenchymal (IPa) delivery of AAV9 encoding representative R→Q variant ZFP-TFs in C57BL/6 mice. The y-axis in each graph is absolute or normalized mRNA expression levels of ZFP-TFs, Mapt, Gfap, Iba1, and NeuN in hippocampal tissue obtained from the right hemisphere of the brains of adult mice at four weeks following treatment with AAV9 (dose—3E10 VG per hemisphere) encoding the ZFP-TF under the human synapsin 1 promoter. Vehicle treatment (VEH) and treatment with a ZFP-TF encoding GFP were used as negative controls. The ZFP-TFs used are indicated in the x-axis. The colored bars represent the mean of values from four mice and the error bars represent standard deviation. The arrow in each figure indicates the group to which other groups' values are normalized.

FIG. 11 is a panel of graphs showing ZFP-TF, human MAPT, mouse Mapt, human STH, GFP, NeuN, and neuroinflammatory marker mRNA expression levels following intraparenchymal (IPa) delivery of AAV9 encoding representative R→Q variant ZFP-TFs or a positive control ZFP-TF construct (57890-T2A-65918) in htau mice. The y-axis in each graph is absolute or normalized mRNA expression levels of ZFP-TFs, human MAPT, mouse Mapt, human STH, GFP, GFAP, IBA1, and NeuN in hippocampal tissue obtained from the right hemisphere of the brains of adult mice at either 3 or 6 months following treatment with AAV9 (dose—3E9, 1E10, or 3E10 VG per hemisphere) encoding the ZFP-TF under the human synapsin 1 promoter. Vehicle treatment (VEH) was used as negative control. The ZFP-TFs, doses, and time points evaluated are indicated on the x-axis. The colored bars represent the mean of values from 5-8 mice per group and the error bars represent the standard deviation.

FIG. 12 is a graph showing human tau protein levels following intraparenchymal (IPa) delivery of AAV9 encoding representative R→Q variant ZFP-TFs or a positive control ZFP-TF construct (57890-T2A-65918) in htau mice. The y-axis in each graph is absolute or normalized total tau protein levels in hippocampal tissue obtained from the right hemisphere of the brains of adult mice at either 3 or 6 months following treatment with AAV9 (dose—3E9, 1E10, or 3E10 VG per hemisphere) encoding the ZFP-TF under the human synapsin 1 promoter. Vehicle treatment (VEH) was used as negative control. The ZFP-TFs, doses, and time points evaluated are indicated on the x-axis. The colored bars represent the mean of values from 5-8 mice per group and the error bars represent the standard deviation.

FIG. 13 is a panel of images from multiplexed in situ hybridization/immunofluorescence staining showing human MAPT transcripts, NeuN protein, and DAPI following intraparenchymal (IPa) delivery of AAV9 encoding representative R→Q variant ZFP-TFs in htau mice. Representative images are shown for hippocampal regions from the left hemisphere of the brains of adult mice at 3 months following treatment with AAV9 (dose—3E9 VG per hemisphere) encoding the ZFP-TF under the human synapsin 1 promoter. Vehicle treatment (VEH) was used as negative control.

FIG. 14 is a table showing exemplary ZFP of the present disclosure. Shown in capital letters are the genomic target sequences (i.e., bound sequences) of the DNA-binding recognition helix sequences that are shown in a single row for each four, five, or six finger ZFP shown (i.e., F1-F4, F1-F5, or F1-F6). This figure also indicates illustrative peptide linker sequences as shown in Table 1 between zinc fingers and between the ZFP domain and the repressor domain for each ZFP shown (i.e., L1, L2, L3, L4, L5, or L6). SEQ ID NO for each sequence is shown in parenthesis.

FIG. 15 is a table showing illustrative full protein sequences for ZFP-TFs comprising the ZFPs shown in FIG. 14. DNA-binding recognition helix sequences are in boldface. Zinc finger linkers are underlined, whereas interdomain linkers are double underlined. SEQ ID NO for each sequence is shown in parenthesis.

FIG. 16 is a table showing exemplary R→Q ZFPs of the present disclosure. Shown in capital letters are the genomic target sequences (i.e., bound sequences) of the DNA-binding recognition helix sequences that are shown in a single row for each four, five, or six finger ZFP shown (i.e., F1-F4, F1-F5, or F1-F6). This figure also indicates illustrative peptide linker sequences as shown in Table 1 between zinc fingers and between the ZFP domain and the repressor domain for each ZFP shown (i.e., L1, L2, L3, L4, L5, or L6). The symbol “\hat{ }” indicates that the arginine (R) residue at the 4th position upstream of the 1st amino acid in the indicated finger is changed to glutamine (Q). SEQ ID NO for each sequence is shown in parenthesis.

FIG. 17 is a table showing illustrative full protein sequences for R→Q ZFP-TFs comprising the ZFPs shown in FIG. 16. DNA-binding recognition helix sequences are in boldface. Zinc finger linkers are underlined, whereas interdomain linkers are double underlined. SEQ ID NO for each sequence is shown in parenthesis.

### DETAILED DESCRIPTION OF THE INVENTION

The present disclosure provides ZFP domains that target sites (i.e., bind DNA sequences) in or near the human MAPT gene. A ZFP domain as described herein may be attached or fused to another functional molecule or domain. The ZFP domains of the present disclosure may be fused to a transcription factor to repress transcription of the human MAPT gene into mRNA. The fusion proteins are called zinc finger protein-transcription factors (ZFP-TFs) that target specifically the human MAPT gene and repress its transcription into RNA. These ZFP-TFs comprise a zinc finger protein (ZFP) domain that binds specifically to a target region in or near the MAPT gene and a transcription repressor domain that reduces the transcription of the gene. Reducing the level of tau in neurons by introducing the ZFP-TFs into the brain of a patient is expected to inhibit (e.g., reduce or stop) the assembly of tau into aggregates and NFTs. Cell-to-cell propagation of tau aggregates will be reduced or prevented. The present ZFP-TFs can be used for the prevention and/or treatment of tauopathies.

The present ZFP-TF approach to tau inhibition has several advantages over the current approaches being tested by others, which include administration of (i) antisense oligonucleotides (ASOs) that bind tau mRNA and prevent its translation and (ii) immunotherapeutic anti-tau antibodies. ZFP-TFs may need to be administered only once (by introducing to the patient a ZFP-TF expression construct), while ASOs require repeated dosing. In addition, the ZFP-TF approach only needs to engage the two alleles of the MAPT gene in the genome of each cell. By contrast, ASOs need to engage numerous copies of the MAPT mRNA in each cell. Additionally, the distribution and tropism of ASOs is fixed, whereas the ZFP-TF approach can be targeted to different cell types and brain regions by altering the promoter, serotype, and route of administration.

The present ZFP-TF approach is advantageous over the antibody approach because antibodies can only bind a subset of tau protein species or conformations. This may not be sufficient for a robust therapeutic effect. In contrast, ZFP-TFs repress tau expression at the DNA level and lead to lower levels of all forms of tau, including different tau conformers and post-translationally modified forms found across tauopathies. ZFP-TFs are therefore agnostic to the form of the toxic species, unlike antibodies. In addition, antibodies are thought to primarily act on extracellular tau, whereas ZFP-TFs can reduce total tau levels inside the cell directly, thereby indirectly lowering extracellular tau levels. Thus, the present ZFP-TF approach is expected to be more effective because tau exerts its pathology intracellularly and the pathogenic species are unknown. Further, antibodies require repeated administration, typically into the periphery, which results in inefficient crossing of the blood-brain barrier, while ZFP-TFs require only a one-time delivery of their expression constructs and can be administered via several routes, including directly to the brain parenchyma, into the CSF, or intravenously.

### I. Targets of the ZFP Domains

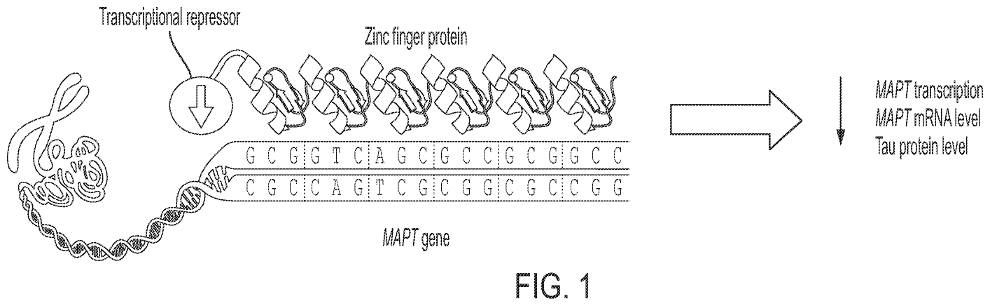

The ZFP domains of the present fusion proteins bind specifically to a target region in or near the human MAPT gene. FIG. 1 illustrates the binding of a ZFP domain to a DNA sequence in the MAPT gene. The ZFP domain in the figure has six zinc fingers; however, as further described below, a ZFP domain that has fewer or more zinc fingers can also be used.

The human MAPT gene spans about 134 kb and has been mapped to chr17q21.31: 45,894,382-46,028,334 (GRCh38.p13). Its nucleotide sequence is available at GenBank accession number ENSG00000186868. The MAPT gene comprises 13-16 exons. Exons 1, 4, 5, 7, 9, 11 and 12 are constitutively expressed whereas exons 2, 3, and 10 can be present in tau protein species derived from alternatively spliced variants, leading to the presence of six different tau protein isoforms in the adult brain. Full-length human tau protein has the following sequence:

(SEQ ID NO: 1; P10636)
MAEPRQEFEV MEDHAGTYGL GDRKDQGGYT
MHQDQEGDTD AGLKESPLQT
PTEDGSEEPG SETSDAKSTP TAEDVTAPLV
DEGAPGKQAA AQPHTEIPEG
TTAEEAGIGD TPSLEDEAAG HVTQEPESGK
VVQEGFLREP GPPGLSHQLM
SGMPGAPLLP EGPREATRQP SGTGPEDTEG
GRHAPELLKH QLLGDLHQEG
PPLKGAGGKE RPGSKEEVDE DRDVDESSPQ
DSPPSKASPA QDGRPPQTAA
REATSIPGFP AEGAIPLPVD FLSKVSTEIP
ASEPDGPSVG RAKGQDAPLE
FTFHVEITPN VQKEQAHSEE HLGRAAFPGA
PGEGPEARGP SLGEDTKEAD
LPEPSEKQPA AAPRGKPVSR VPQLKARMVS
KSKDGTGSDD KKAKTSTRSS
AKTLKNRPCL SPKHPTPGSS DPLIQPSSPA
VCPEPPSSPK YVSSVTSRTG
SSGAKEMKLK GADGKTKIAT PRGAAPPGQK
GQANATRIPA KTPPAPKTPP
SSGEPPKSGD RSGYSSPGSP GTPGSRSRTP
SLPTPPTREP KKVAVVRTPP
KSPSSAKSRL QTAPVPMPDL KNVKSKIGST
ENLKHQPGGG KVQIINKKLD
LSNVQSKCGS KDNIKHVPGG GSVQIVYKPV
DLSKVTSKCG SLGNIHHKPG
GGQVEVKSEK LDFKDRVQSK IGSLDNITHV
PGGGNKKIET HKLTFRENAK
AKTDHGAEIV YKSPVVSGDT SPRHLSNVSS
TGSIDMVDSP QLATLADEVS
ASLAKQGL

The DNA-binding ZFP domains of the ZFP-TFs direct the fusion proteins to a target region of the MAPT gene and bring the transcriptional repression domains of the fusion proteins to the target region. The repression domains recruit transcriptional co-repressor complexes to modify the chromatin into a non-permissive state for transcription by RNA Polymerase II. The target region for the ZFP-TFs can be any suitable site in or near the MAPT gene that allows repression of gene expression. By way of example, the target region includes, or is adjacent to (either downstream or upstream of) a MAPT transcription start site (TSS) or a MAPT transcription regulatory element (e.g., promoter, enhancer, RNA polymerase pause site, and the like).

In some embodiments, the genomic target region is at least 8 bps in length. For example, the target region may be 8 bps to 40 bps in length, such as 12, 15, 16, 17, 18, 19, 20, 21, 24, 27, 30, 33, or 36 bps in length. The targeted sequence may be on the sense strand of the gene, or the antisense strand of the gene. To ensure targeting accuracy and to reduce off-target binding or activity by the ZFP-TFs, the sequence of the selected MAPT target region preferably has less than 75% homology (e.g., less than 70%, less than 65%, less than 60%, or less than 50%) to sequences in other genes. In certain embodiments, the target region of the present ZFP-TFs is 12-20 (e.g., 12-18, 15-19, 15, 18, or 19) bps in length and resides within 1500 bps upstream to 1000 bps downstream (e.g., −1000 bps to +1000 bps, +750, or +500 bps) of the TSS.

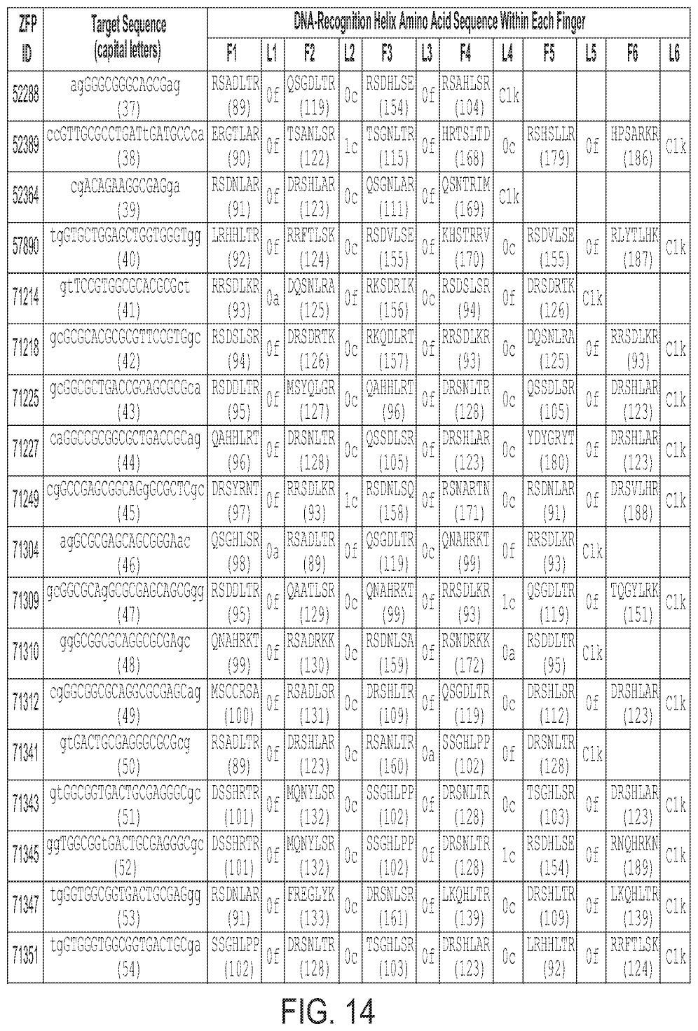

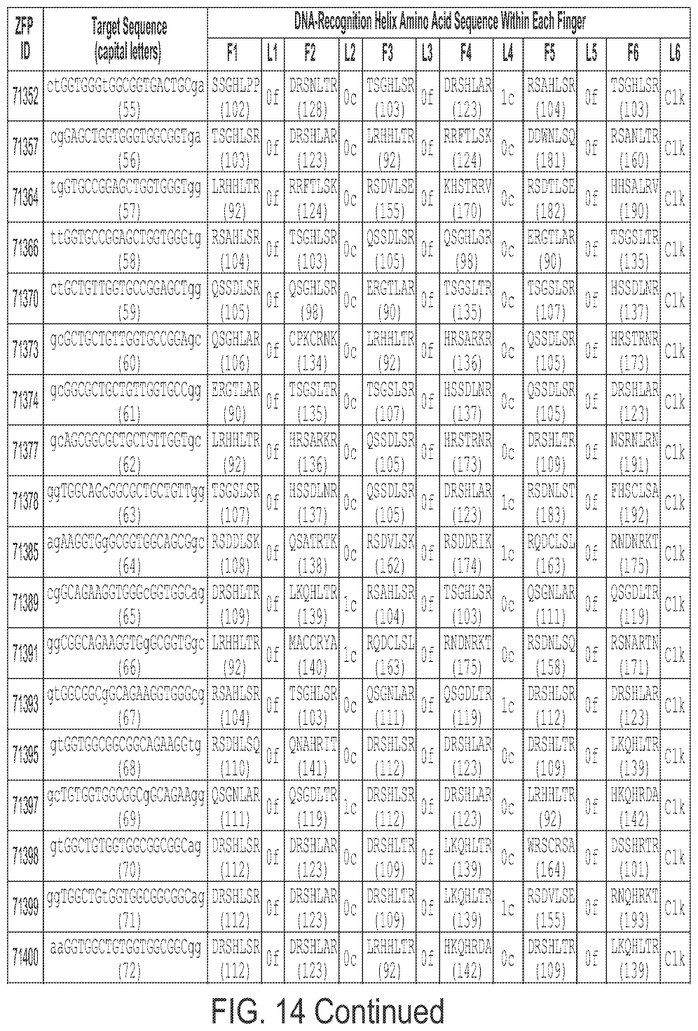

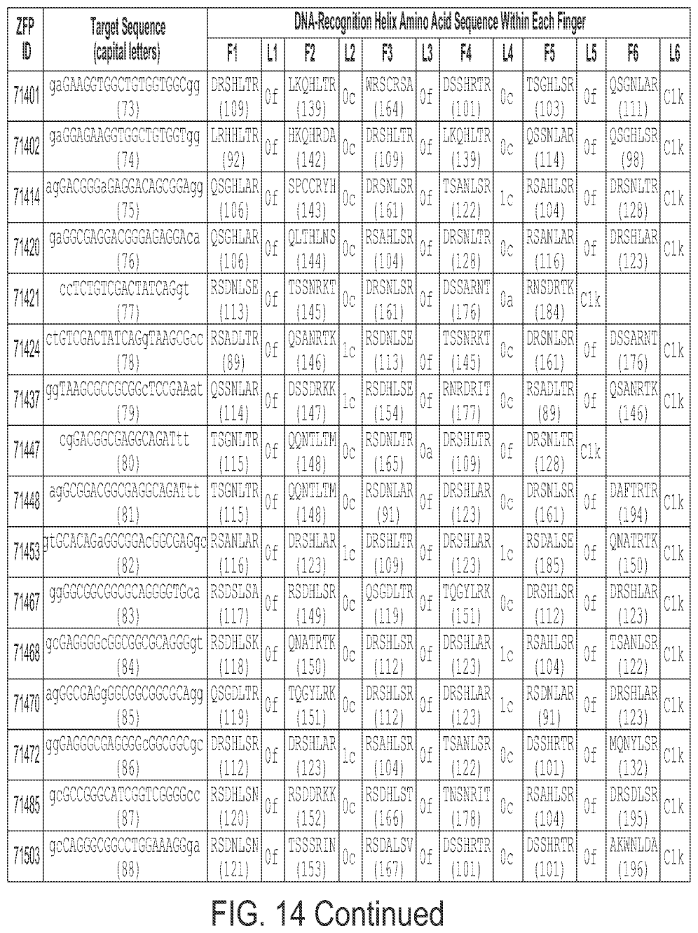

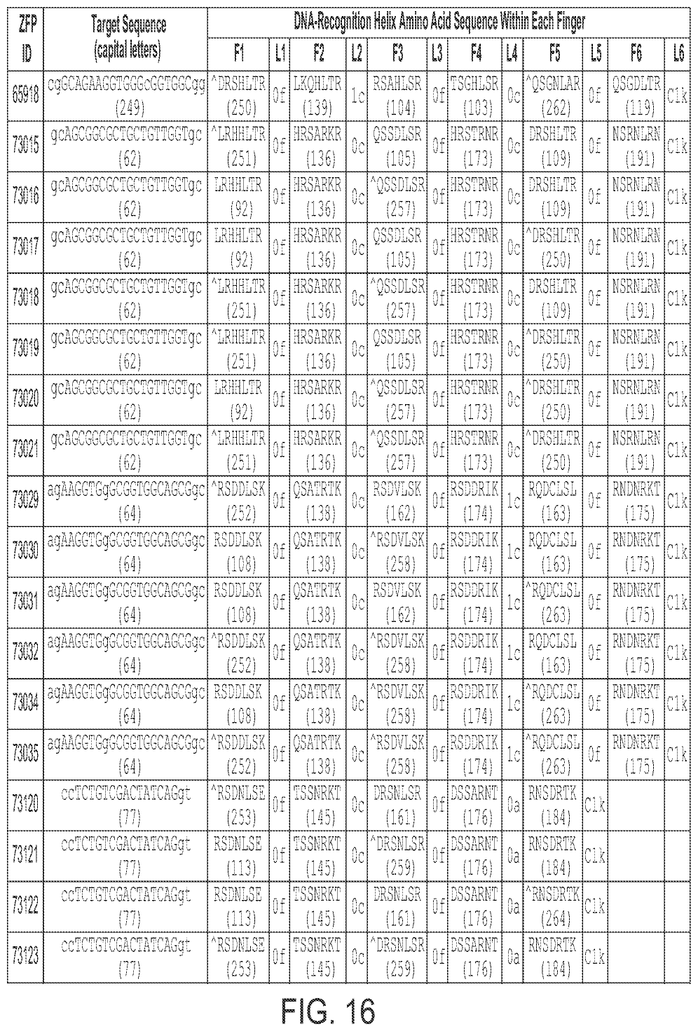

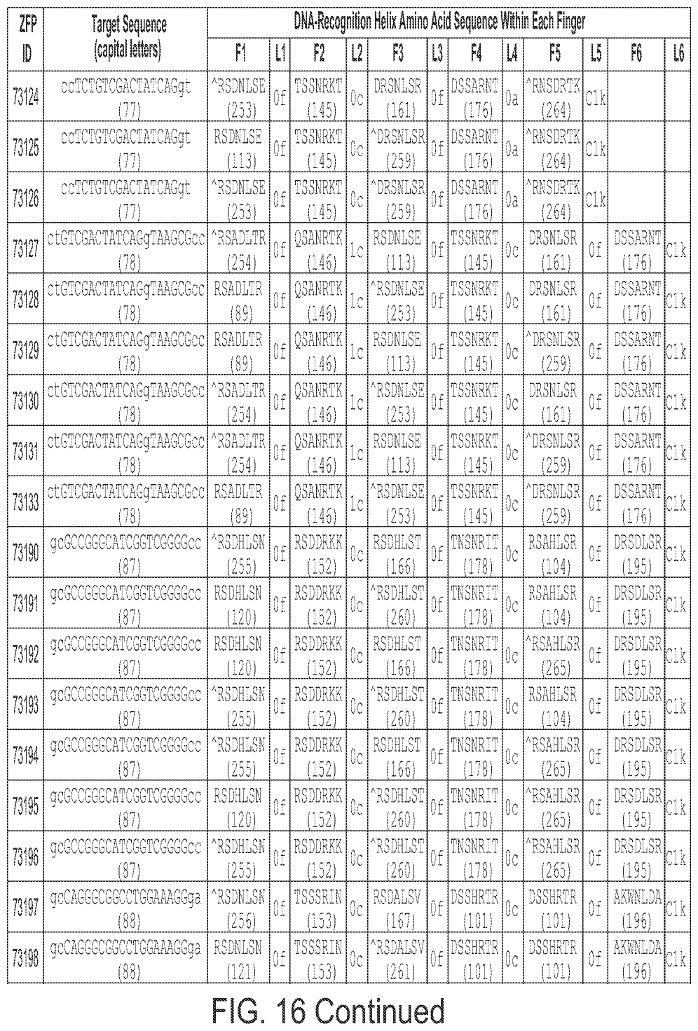

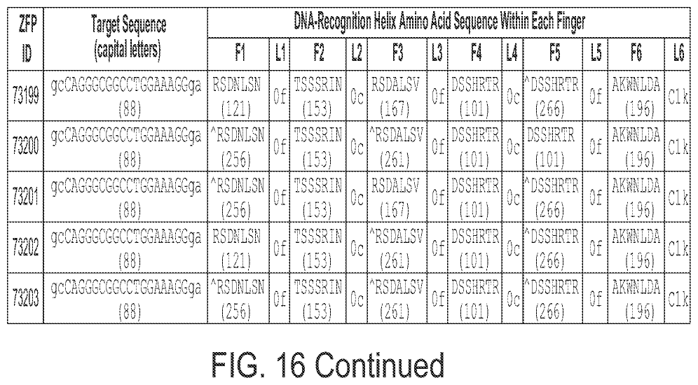

In some embodiments, the present engineered ZFPs bind to a target site (i.e., Target Sequence) as shown in a single row of FIG. 14 or FIG. 16, preferably with no or little detectable off-target binding or activity, including contiguous or non-contiguous sequences within these target sites. In some embodiments, the target site comprises and/or is within any one of SEQ ID NOS: 37-88, and 249.

Other criteria for further evaluating target segments include the prior availability of ZFPs binding to such segments or related segments, ease of designing new ZFPs to bind a given target segment, and off-target binding risk.

### II. Zinc Finger Protein Domains

A “zinc finger protein” or “ZFP” refers to a protein having a DNA-binding domain that is stabilized by zinc. ZFPs bind to DNA in a sequence-specific manner. The individual DNA-binding unit of a ZFP is referred to as a “zinc finger.” Each finger contains a DNA-binding “recognition helix” that is typically comprised of seven amino acid residues and determines DNA binding specificity. A ZFP domain has at least one finger and each finger binds from two to four base pairs of nucleotides, typically three or four base pairs of DNA (contiguous or noncontiguous). Each zinc finger typically comprises approximately 30 amino acids and chelates zinc. An engineered ZFP can have a novel binding specificity, compared to a naturally occurring ZFP. Engineering methods include, but are not limited to, rational design and various types of selection. Rational design includes, for example, using databases comprising triplet (or quadruplet) nucleotide sequences and individual zinc finger amino acid sequences, in which each triplet or quadruplet nucleotide sequence is associated with one or more amino acid sequences of zinc fingers that bind the particular triplet or quadruplet sequence. See, e.g., ZFP design methods described in detail in U.S. Pat. Nos. 5,789,538; 5,925,523; 6,007,988; 6,013,453; 6,140,081; 6,200,759; 6,453,242; 6,534,261; 6,979,539; and 8,586,526; and International Patent Publications WO 95/19431; WO 96/06166; WO 98/53057; WO 98/53058; WO 98/53059; WO 98/53060; WO 98/54311; WO 00/27878; WO 01/60970; WO 01/88197; WO 02/016536; WO 02/099084; and WO 03/016496. A ZFP domain as described herein may be attached or fused to another molecule (e.g., domain), for example, a protein. Such ZFP-fusions may comprise a domain that enables gene activation (e.g., activation domain), gene repression (e.g., repression domain), ligand binding (e.g., ligand-binding domain), high-throughput screening (e.g., ligand-binding domain), localized hypermutation (e.g., activation-induced cytidine deaminase domain), chromatin modification (e.g., histone deacetylase domain), recombination (e.g., recombinase domain), targeted integration (e.g., integrase domain), DNA modification (e.g., DNA methyl-transferase domain), base editing (e.g., base editor domain), or targeted DNA cleavage (e.g., nuclease domain). Examples of engineered ZFP domains are shown in FIG. 14 and FIG. 16.

The ZFP domain of the present engineered ZFP fusions may include at least one (e.g., one, two, three, four, five, six, seven, eight, nine, ten, eleven, twelve, thirteen, or more) zinc finger(s). A ZFP domain having one finger typically recognizes a target site that includes 3 or 4 nucleotides. A ZFP domain having two fingers typically recognizes a target site that includes 6 or 8 nucleotides. A ZFP domain having three fingers typically recognizes a target site that includes 9 or 12 nucleotides. A ZFP domain having four fingers typically recognizes a target site that includes 12 to 15 nucleotides. A ZFP domain having five fingers typically recognizes a target site that includes 15 to 18 nucleotides. A ZFP domain having six fingers can recognize target sites that include 18 to 21 nucleotides.

In some embodiments, the present engineered ZFPs comprise a DNA-binding recognition helix sequence having at least 4 of the amino acids of any recognition helix as shown in FIG. 14 or FIG. 16. In other embodiments, the present engineered ZFPs comprise a DNA-binding recognition helix sequence shown in FIG. 14 or FIG. 16. For example, an engineered ZFP may comprise the sequence of F1, F2, F3, F4, F5, or F6 as shown in FIG. 14 or FIG. 16.

In some embodiments, the present engineered ZFPs comprise two adjacent DNA-binding recognition helix sequences shown in a single row of FIG. 14 or FIG. 16. For example, an engineered ZFP may comprise the sequences of F1-F2, F2-F3, F3-F4, F4-F5, or F5-F6 as shown in a single row of FIG. 14 or FIG. 16.

In some embodiments, the present engineered ZFPs comprise the DNA-binding recognition helix sequences shown in a single row of FIG. 14 or FIG. 16. For example, an engineered ZFP may comprise the sequences of F1, F2, F3, F4, F5, and F6 (e.g., F1-F4, F1-F5, or F1-F6) as shown in a single row of FIG. 14 or FIG. 16.

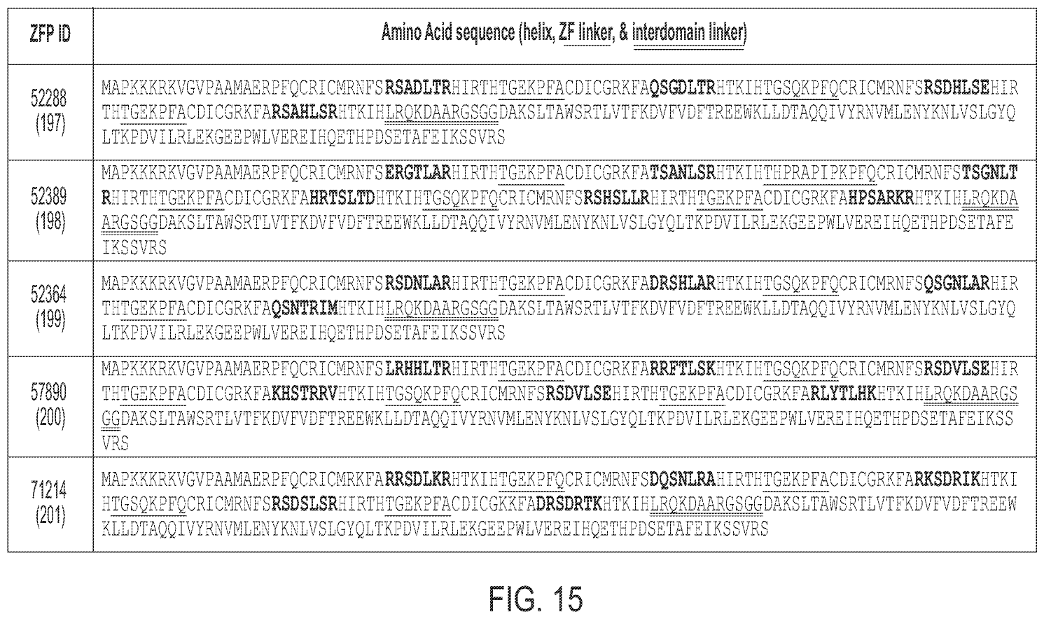

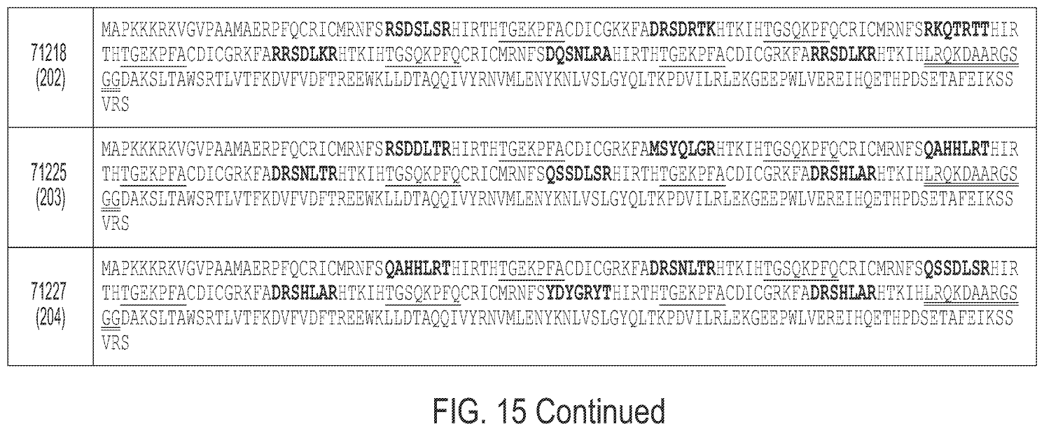

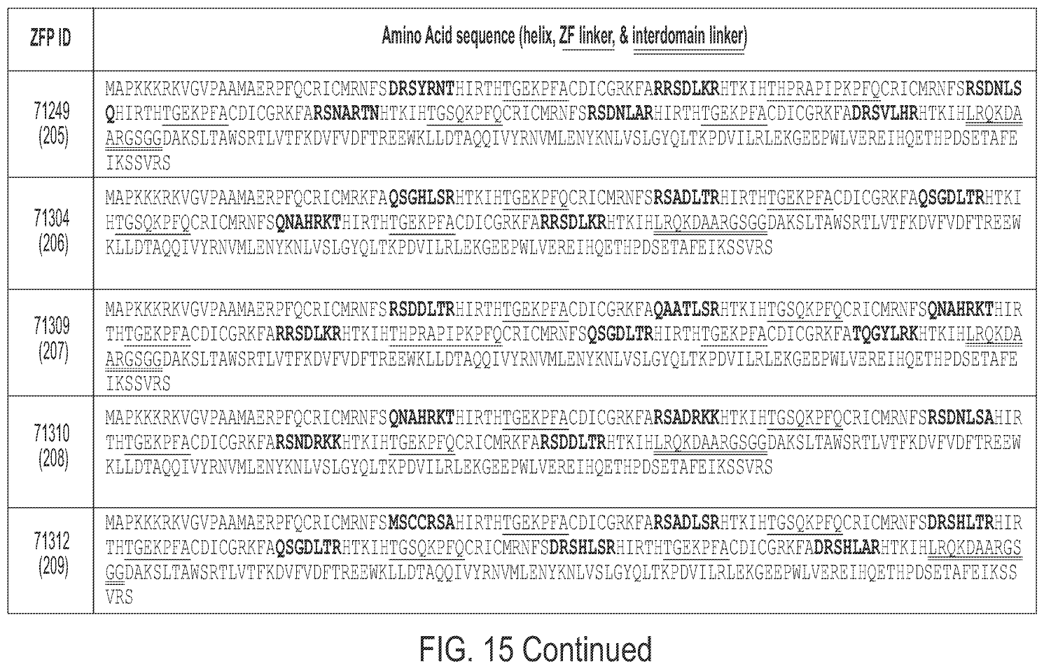

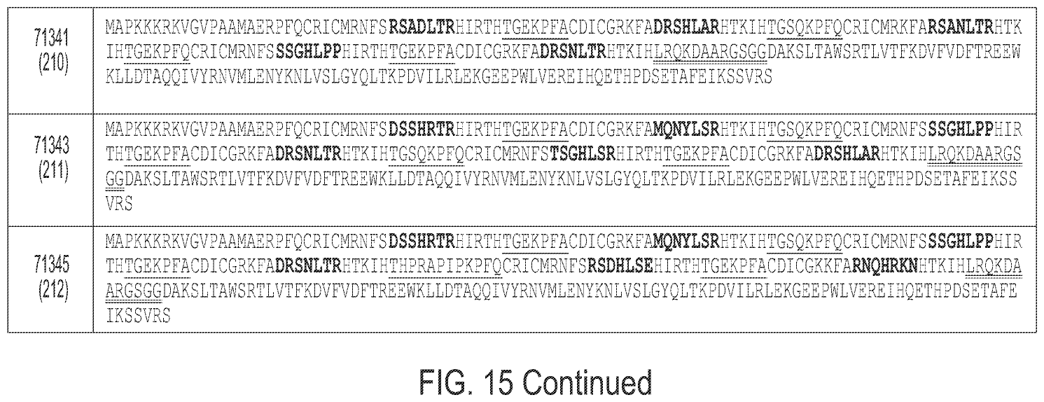

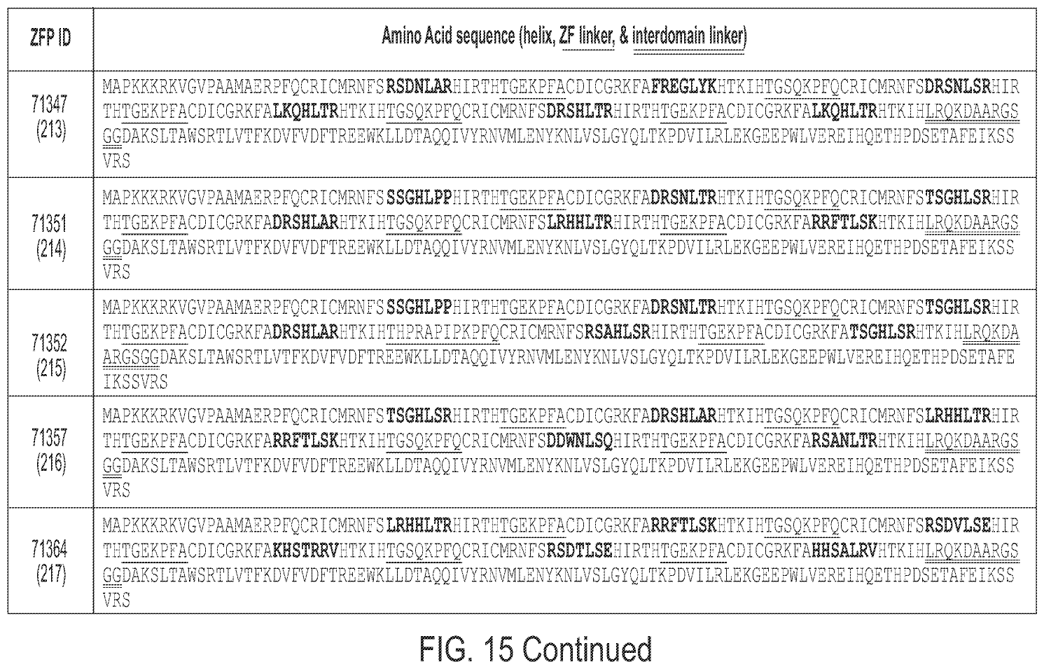

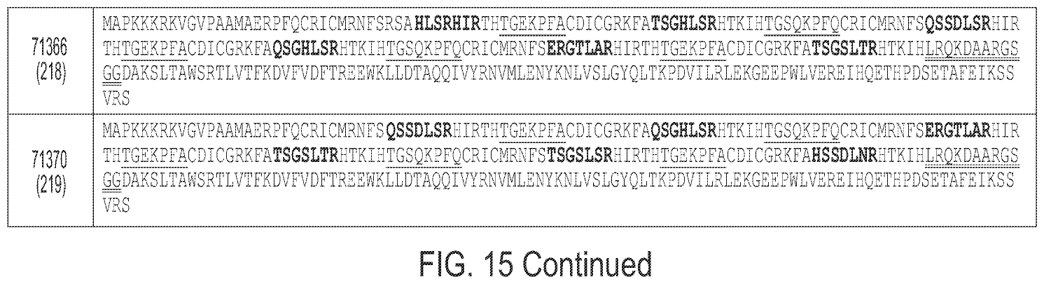

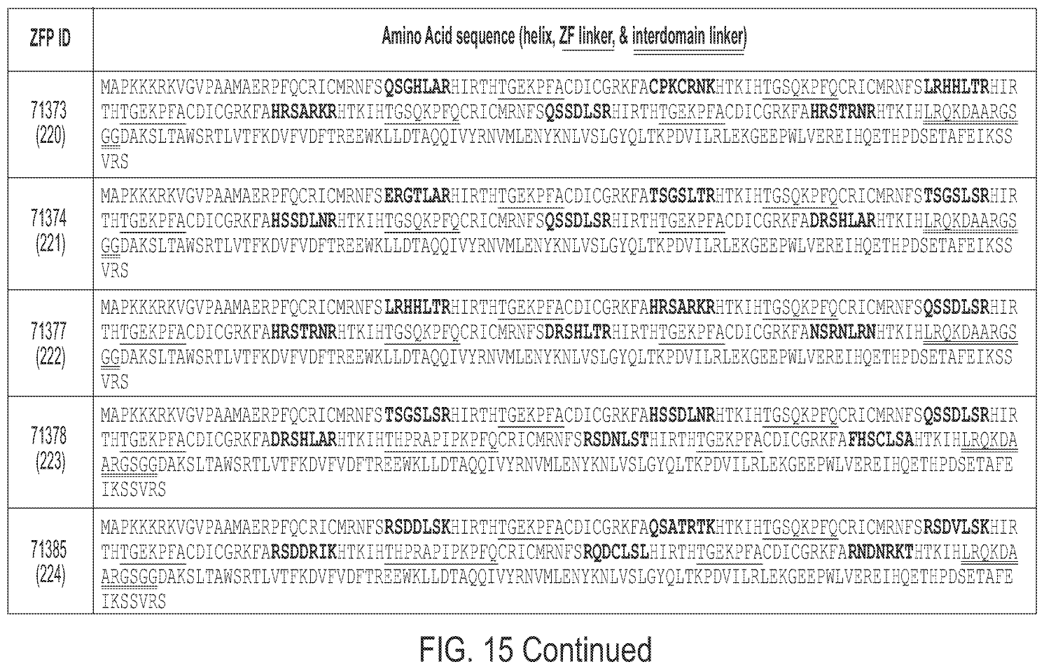

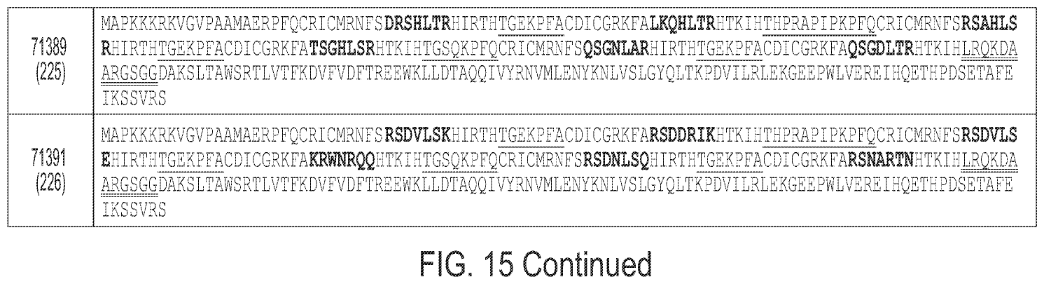

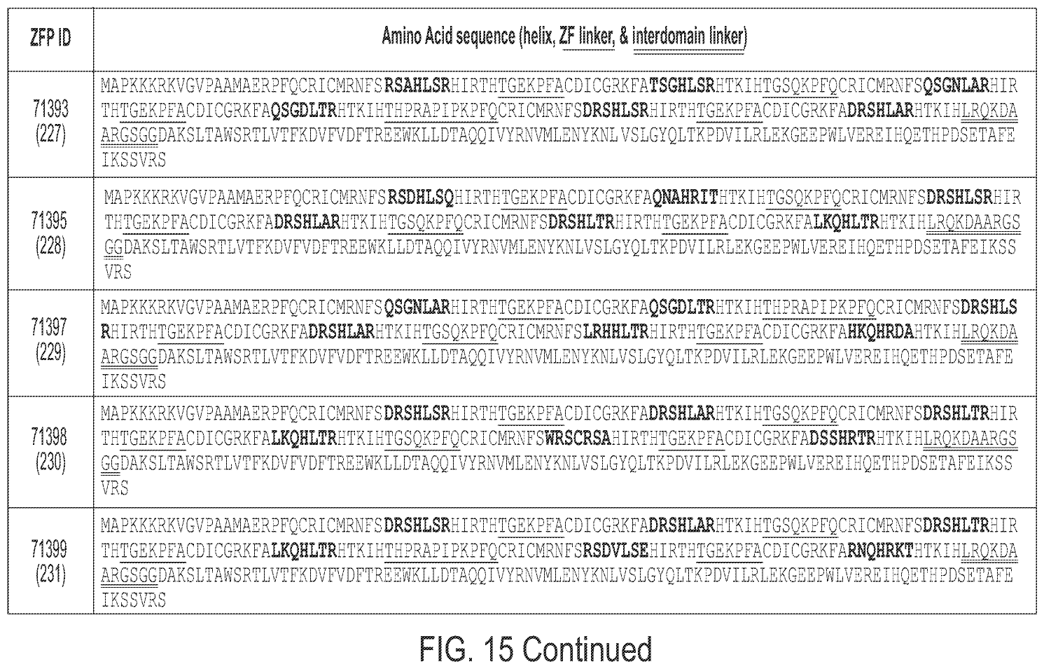

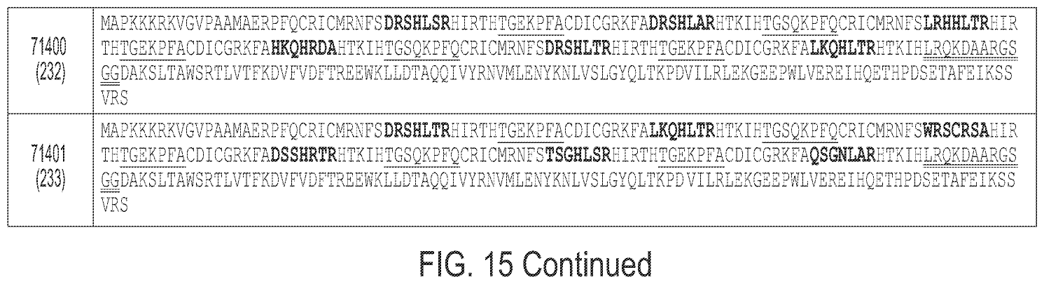

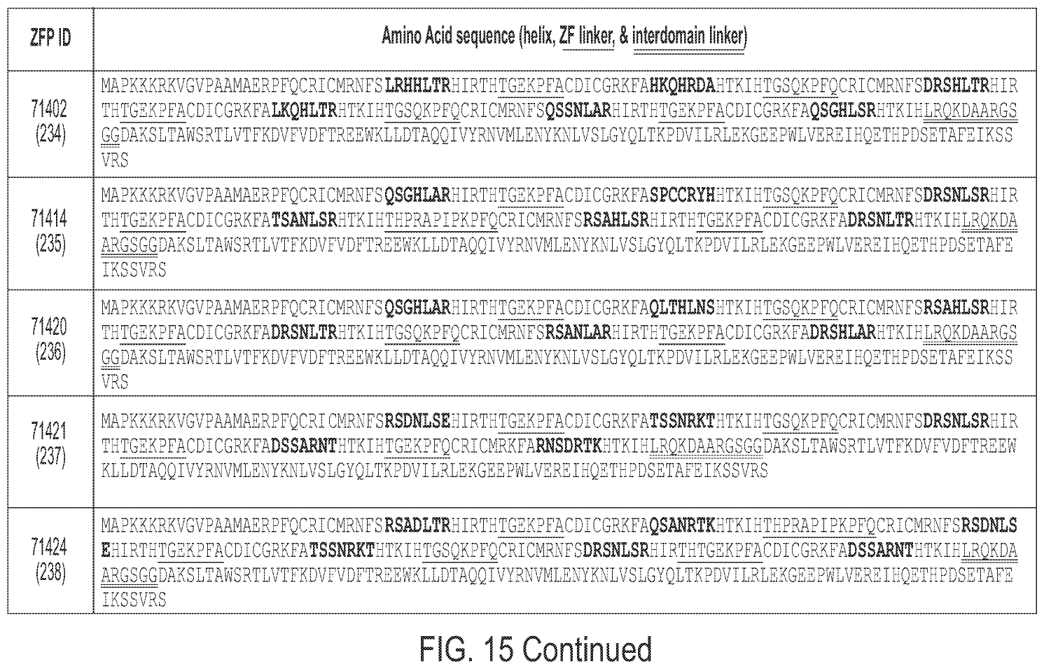

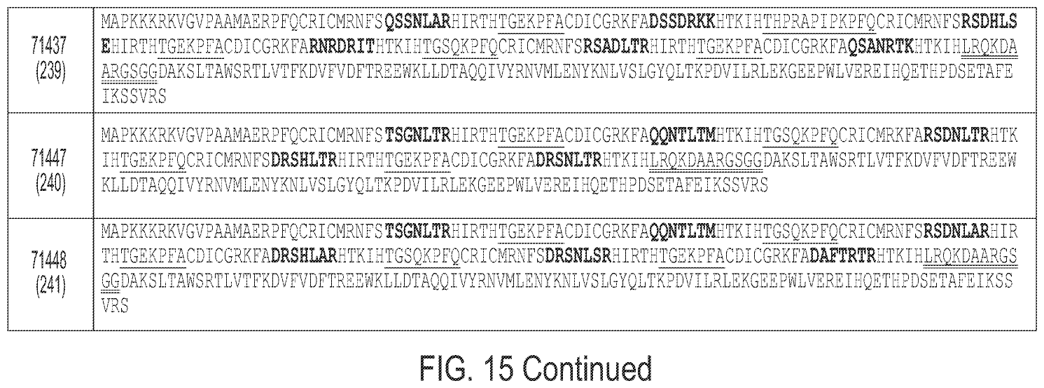

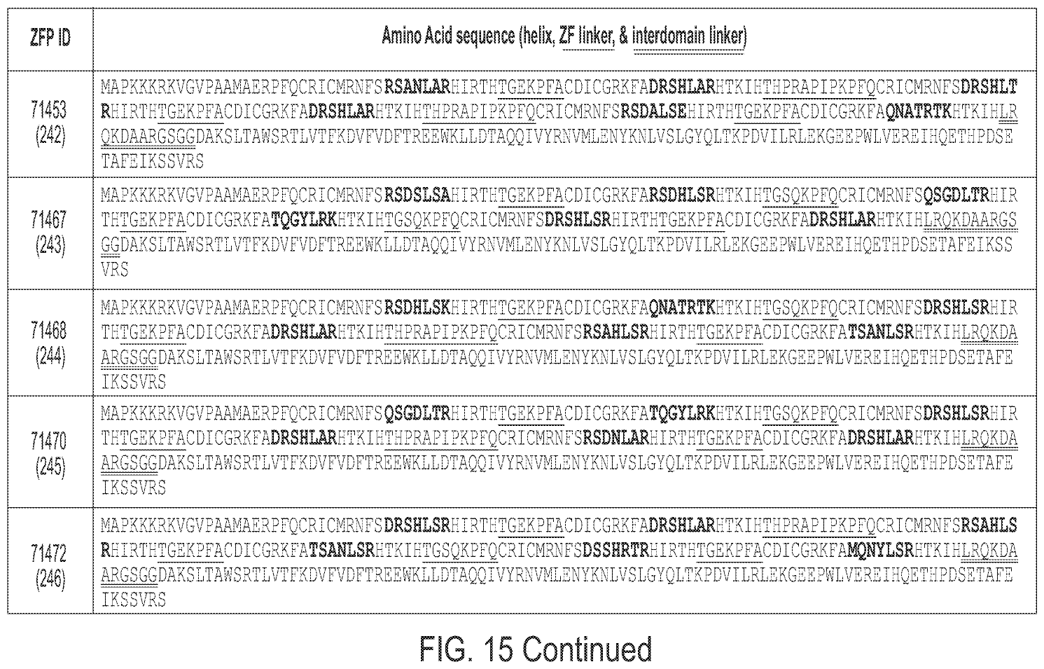

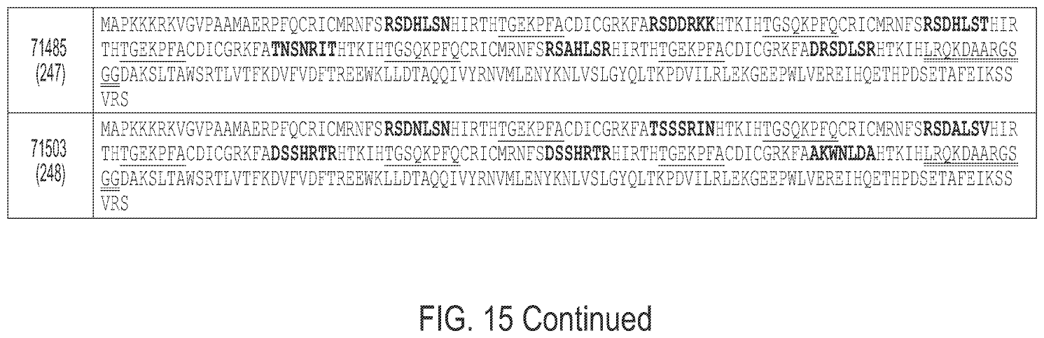

In some embodiments, an engineered ZFP described herein comprises the recognition helix and backbone portions of a sequence shown in a single row of FIG. 15. In some embodiments, an engineered ZFP described herein comprises the recognition helix and backbone portions of a sequence shown in a single row of FIG. 15 as the sequence would appear following post-translational modification. For example, post-translational modification may remove the initiator methionine residue from a sequence as shown in FIG. 15.

The target specificity of the ZFP domain may be improved by mutations to the ZFP backbone sequence as described in, e.g., U.S. Pat. Pub. 2018/0087072. The mutations include those made to residues in the ZFP backbone that can interact non-specifically with phosphates on the DNA backbone but are not involved in nucleotide target specificity. In some embodiments, these mutations comprise mutating a cationic amino acid residue to a neutral or anionic amino acid residue. In some embodiments, these mutations comprise mutating a polar amino acid residue to a neutral or non-polar amino acid residue. In further embodiments, mutations are made at positions (−4), (−5), (−9) and/or (−14) relative to the DNA-binding helix. In some embodiments, a zinc finger may comprise one or more mutations at positions (−4), (−5), (−9) and/or (−14). In further embodiments, one or more zinc fingers in a multi-finger ZFP domain may comprise mutations at positions (−4), (−5), (−9) and/or (−14). In some embodiments, the amino acids at positions (−4), (−5), (−9) and/or (−14) (e.g., an arginine (R) or lysine (K)) are mutated to an alanine (A), leucine (L), serine (S), aspartate (D), glutamate (E), tyrosine (Y), and/or glutamine (Q). In some embodiments, the R residue at position (−5) is mutated to Q. The symbol “\hat{ }” in FIG. 16 indicates that the arginine (R) residue at the 4th position upstream of the 1st amino acid in the indicated recognition helix is changed to glutamine (Q). In each recognition helix sequence, the positions of the seven DNA-binding amino acids are numbered −1, +1, +2, +3, +4, +5, and +6. Thus, the position for the R-to-Q substitution is numbered as (−5).

In some embodiments, the present engineered ZFPs comprise a DNA-binding recognition helix sequence and associated backbone mutation as shown in FIG. 16. In some embodiments, the present engineered ZFPs comprise the DNA-binding recognition helix sequences and associated backbone mutations as shown in a single row of FIG. 16.

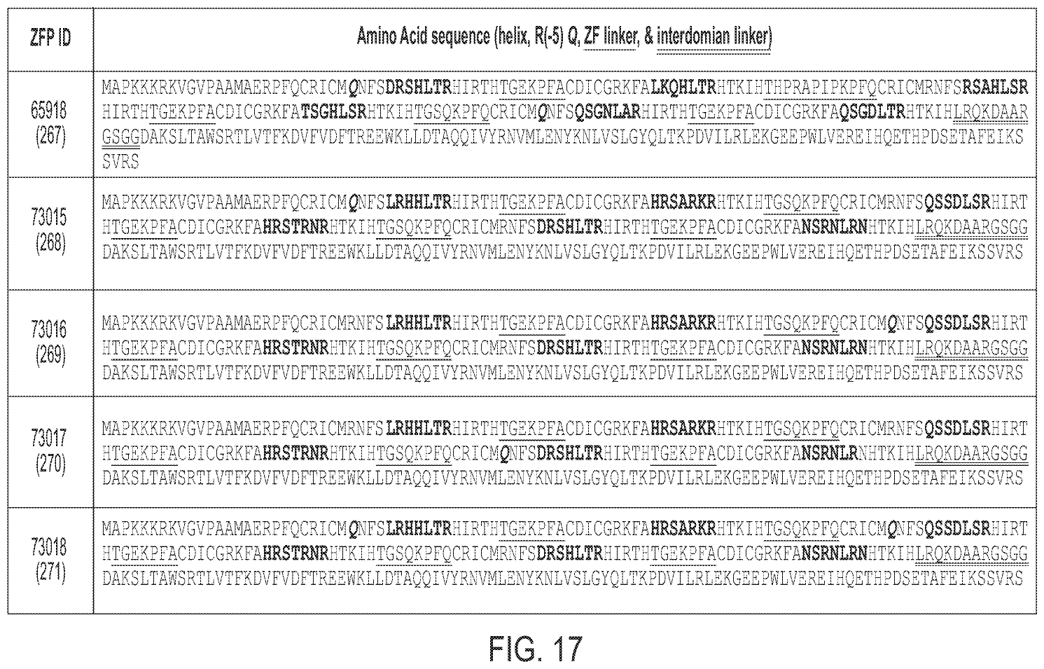

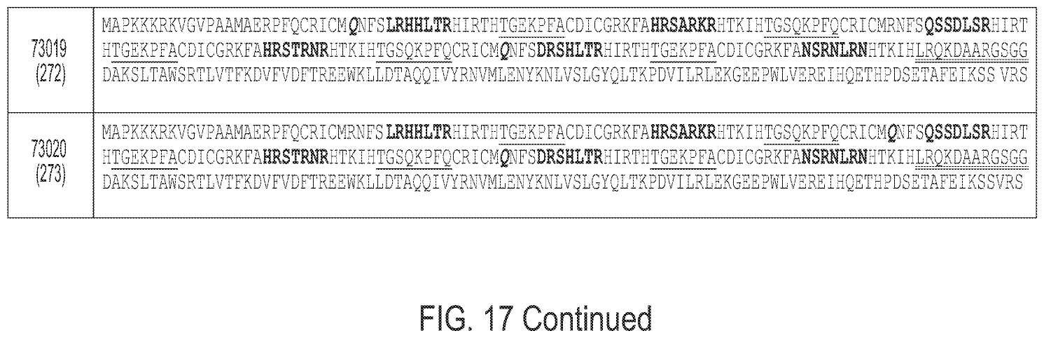

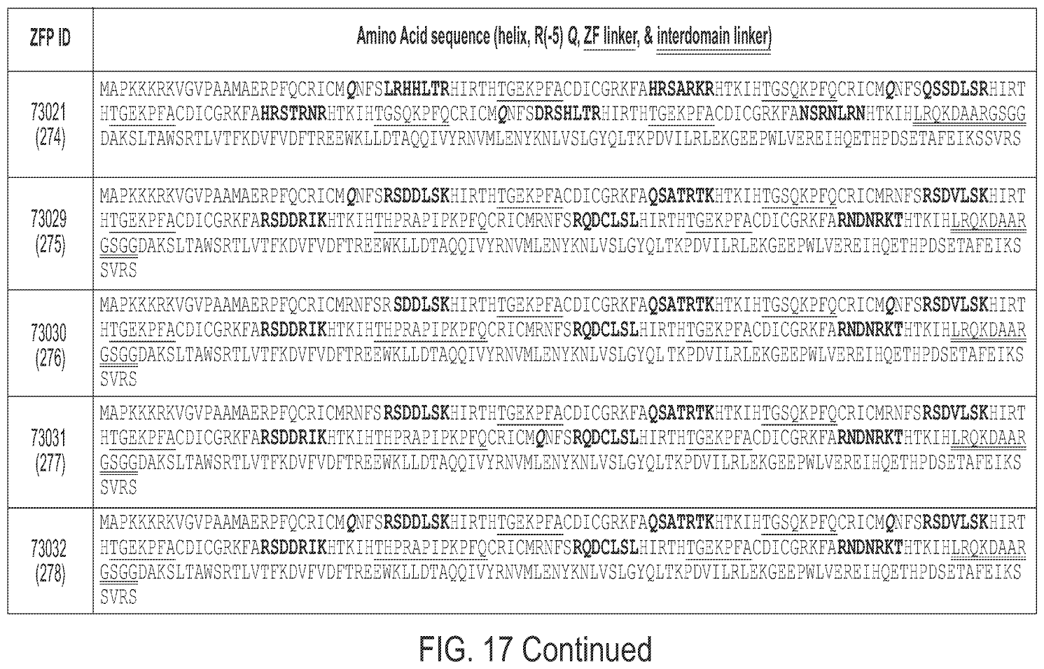

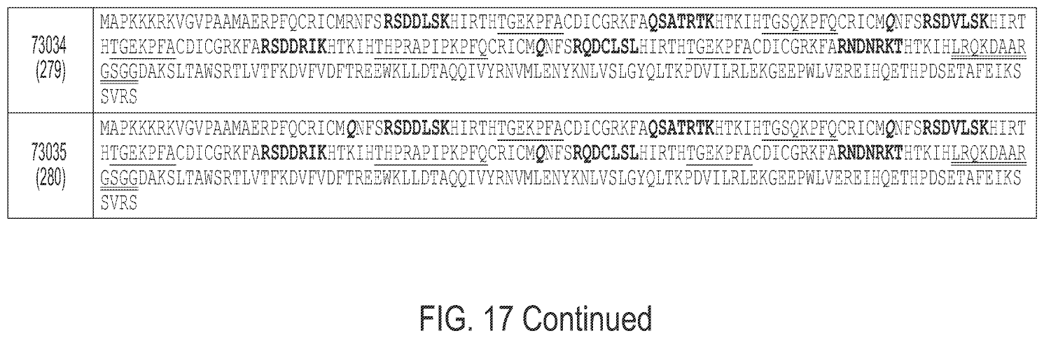

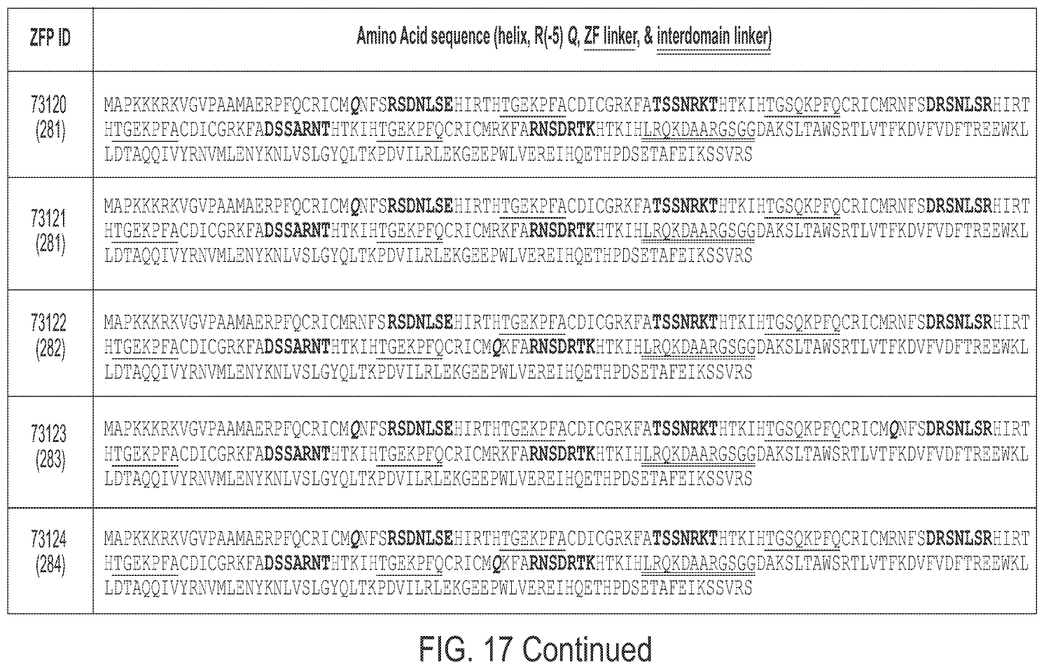

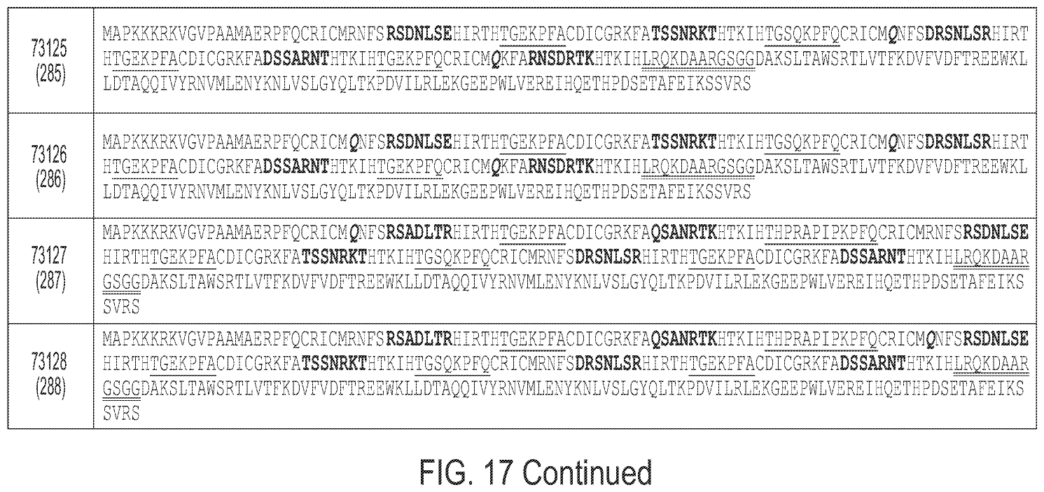

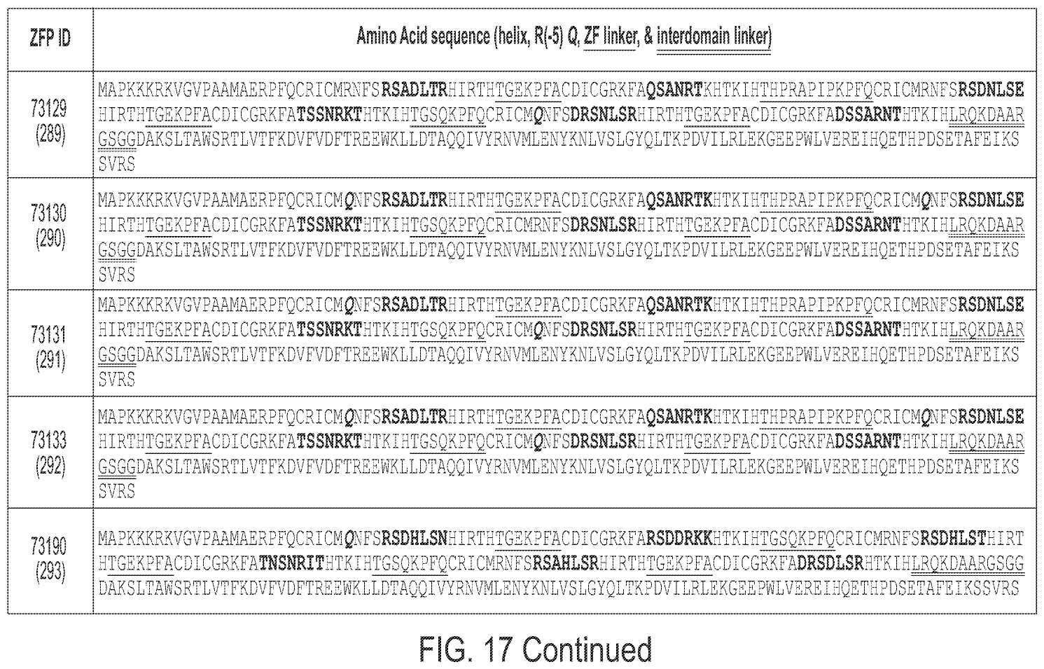

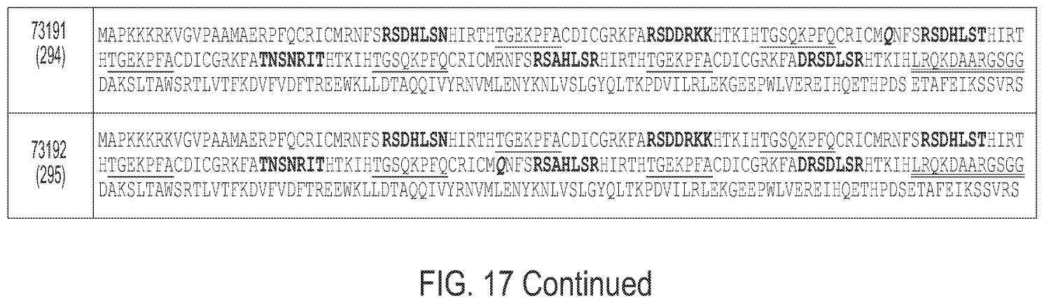

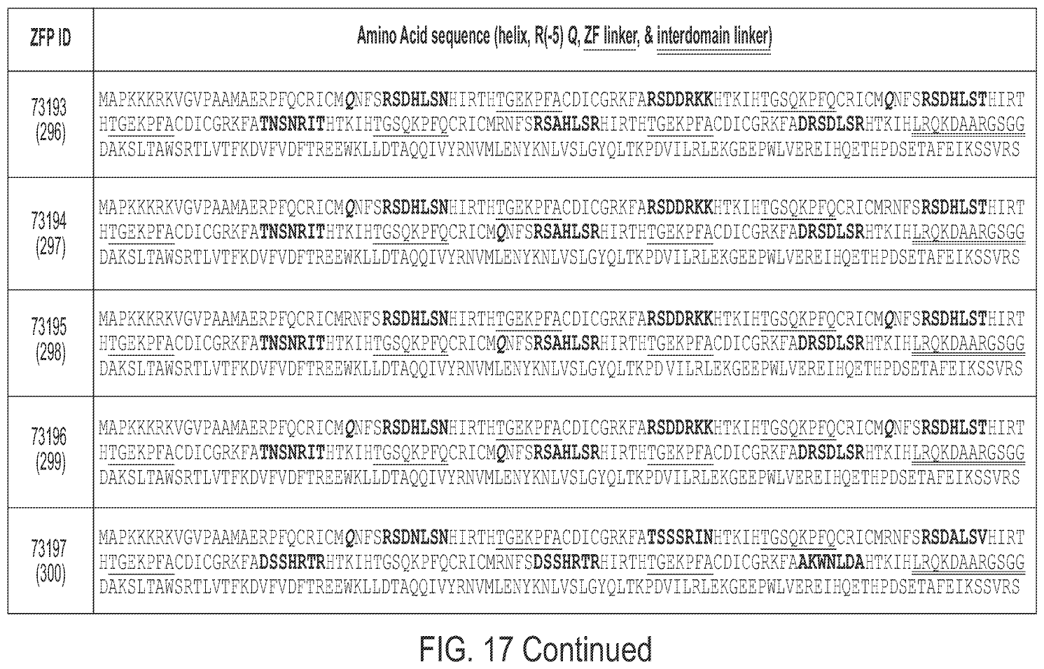

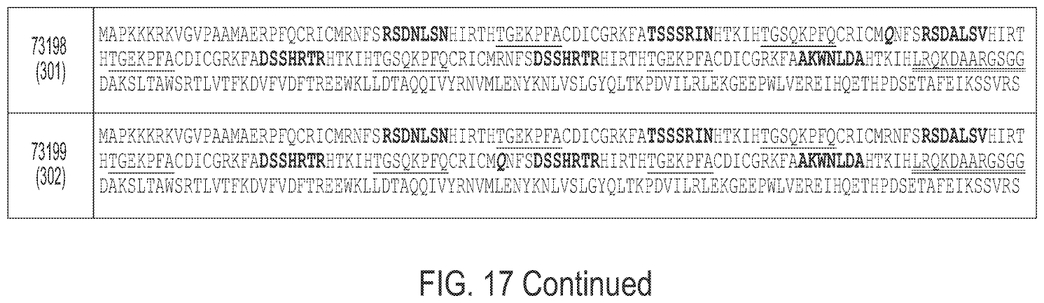

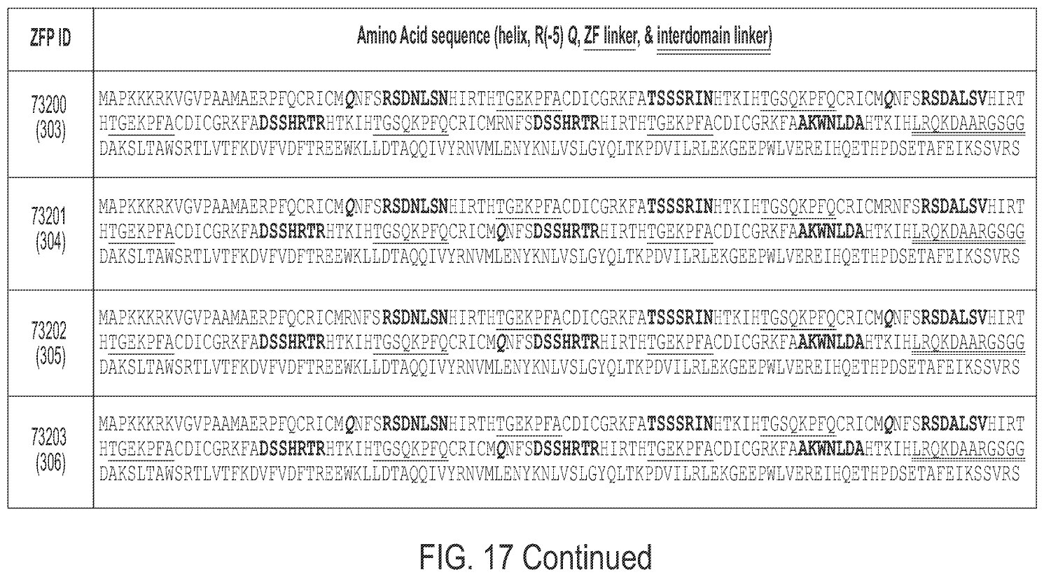

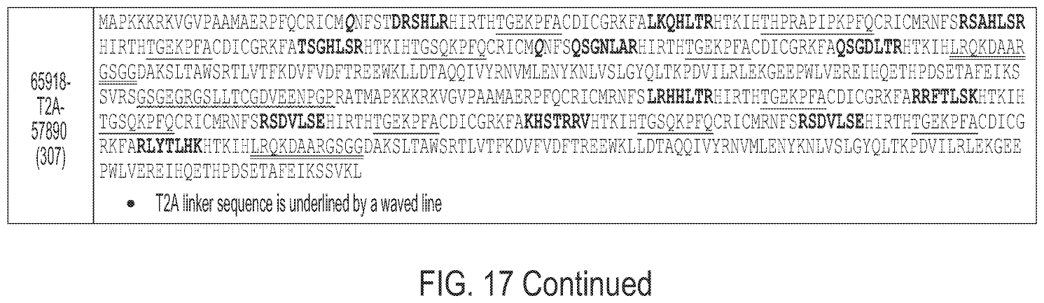

In some embodiments, an engineered ZFP described herein comprises the recognition helix and backbone portions of a sequence shown in a single row of FIG. 17. In some embodiments, an engineered ZFP described herein comprises the recognition helix and backbone portions of a sequence shown in a single row of FIG. 17 as the sequence would appear following post-translational modification. For example, post-translational modification may remove the initiator methionine residue from a sequence as shown in FIG. 17.

### III. Zinc Finger Protein Transcription Factors

The ZFP domains described herein may be fused to a transcription factor. In some embodiments, the present fusion proteins contain a DNA-binding zinc finger protein (ZFP) domain and a transcription factor domain (i.e., ZFP-TF). In some embodiments, the transcription factor may be a transcription repressor domain, wherein the ZFP and repressor domains may be associated with each other by a direct peptidyl linkage or a peptide linker, or by dimerization (e.g., through a leucine zipper, a STAT protein N terminal domain, or an FK506 binding protein). As used herein, a “fusion protein” refers to a polypeptide with covalently linked domains as well as a complex of polypeptides associated with each other through non-covalent bonds. The transcription repressor domain can be associated with the ZFP domain at any suitable position, including the C- or N-terminus of the ZFP domain.

In some embodiments, the present ZFP-TFs bind to their target with a K_{D} of less than about 25 nM and repress transcription of a human MAPT gene by 20% or more (e.g., by 30%, 40%, 50%, 60%, 70%, 80%, 90%, 95%, 99%, or more). In some embodiments, two or more of the present ZFP-TFs are expressed in a cell to synergistically modulate MAPT expression in the cell (see, e.g., U.S. Patent Application Publication Nos. 2020/0101133 and 2020/0109406). Such synergy ZFPs may be linked by a 2A linker peptide, e.g., T2A GSGEGRGSLLTCGDVEENPGP (SEQ ID NO:19). Thus, two or more of the present ZFP-TFs may be used concurrently in a patient, where the ZFP-TFs bind to different target regions in the MAPT gene, so as to achieve optimal repression of MAPT expression.

In some embodiments, the present ZFP-TFs comprise one or more zinc finger domains. The domains may be linked together via an extendable flexible linker such that, for example, one domain comprises one or more (e.g., 4, 5, or 6) zinc fingers and another domain comprises additional one or more (e.g., 4, 5, or 6) zinc fingers. In some embodiments, the linker is a standard inter-finger linker such that the finger array comprises one DNA-binding domain comprising 8, 9, 10, 11 or 12 or more fingers. In other embodiments, the linker is an atypical linker such as a flexible linker. For example, two ZFP domains may be linked to a transcription repressor TF in the configuration (from N terminus to C terminus) ZFP-ZFP-TF, TF-ZFP-ZFP, ZFP-TF-ZFP, or ZFP-TF-ZFP-TF (two ZFP-TF fusion proteins are fused together via a linker).

In some embodiments, the ZFP-TFs are “two-handed,” i.e., they contain two zinc finger clusters (two ZFP domains) separated by intervening amino acids so that the two ZFP domains bind to two discontinuous target sites. An example of a two-handed type of zinc finger binding protein is SIP1, where a cluster of four zinc fingers is located at the amino terminus of the protein and a cluster of three fingers is located at the carboxyl terminus (see Remade et al., EMBO J. (1999) 18(18):5073-84). Each cluster of zinc fingers in these proteins is able to bind to a unique target sequence and the spacing between the two target sequences can comprise many nucleotides.

In some embodiments, an engineered ZFP-TF described herein binds to a target site as shown in a single row of FIG. 14 or FIG. 16, preferably with no or little detectable off-target binding or activity. Off-target binding may be determined, for example, by measuring the activity of ZFP-TFs at off-target genes. In some embodiments, an engineered ZFP-TF described herein comprises a DNA-binding recognition helix sequence shown in FIG. 14 or FIG. 16. In some embodiments, an engineered ZFP-TF described herein comprises two adjacent DNA-binding recognition helix sequences shown in a single row of FIG. 14 or FIG. 16. In some embodiments, an engineered ZFP-TF described herein comprises the DNA-binding recognition helix sequences shown in a single row of FIG. 14 or FIG. 16.

A. Transcription Repressor Domains

The present ZFP-TFs comprise an engineered ZFP domain as described herein and one or more transcription repressor domains that dampen the transcription activity of the MAPT gene. One or more engineered ZFP domains and one or more transcription repressor domains may be joined by a flexible linker. Non-limiting examples of transcription repressor domains are the KRAB domain of KOX1 or ZIM3 (or any other KRAB domain containing protein. See, e.g., Alerasool et al., Nature Methods (2020) 17:1093-6), KAP-1, MAD, FKHR, EGR-1, ERD, SID, TGF-beta-inducible early gene (TIEG), v-ERB-A, MBD2, MBD3, TRa, histone methyltransferase, histone deacetylase (HDAC), nuclear hormone receptor (e.g., estrogen receptor or thyroid hormone receptor), members of the DNMT family (e.g., DNMT1, DNMT3A, DNMT3B), Rb, and MeCP2. See, e.g., Bird et al., Cell (1999) 99:451-4; Tyler et al., Cell (1999) 99:443-6; Knoepfler et al., Cell (1999) 99:447-50; and Robertson et al., Nature Genet. (2000) 25:338-42. Additional exemplary repression domains include, but are not limited to, ROM2 and AtHD2A. See, e.g., Chem et al., Plant Cell (1996) 8:305-21; and Wu et al., Plant J. (2000) 22:19-27.

In some embodiments, the transcription repressor domain comprises a sequence from the Kruppel-associated box (KRAB) domain of the human zinc finger protein 10/KOX1 (ZNF10/KOX1) (e.g., GenBank No. NM_015394.4). An exemplary KRAB domain sequence is:

(SEQ ID NO: 2)
DAKSLTAWSR TLVTFKDVFV DFTREEWKLL DTAQQIVYRN
VMLENYKNLV SLGYQLTKPD VILRLEKGEE PWLVEREIHQ
ETHPDSETAF EIKSSV

Variants of this KRAB sequence may also be used so long as they have the same or similar transcription repressor function.

B. Peptide Linkers

The ZFP domain and the transcription repressor domain of the present ZFP-TFs and/or the zinc fingers within the ZFP domains may be linked through a peptide linker, e.g., a noncleavable peptide linker of about 5 to 200 amino acids (e.g., 5, 6, 7, 8, 9, 10, 11, 12, 13, 14, 15, 16, 17, 18, 19, 20 or more amino acids). Preferred linkers are typically flexible amino acid subsequences that are synthesized as a recombinant fusion protein. In some embodiments, zinc fingers are linked such that there is no gap between the linked module target subsites in the target nucleic acid molecule. In other embodiments, zinc fingers are linked by linkers designed to allow the linked modules to bind to target sites with 1, 2 or 3 base pair gaps between the linked module target subsites in the target nucleic acid molecule. See, e.g., U.S. Pat. No. 8,772,453.

In some embodiments, the peptide linker is three to 20 amino acid residues in length and is rich in G and/or S. Non-limiting examples of such linkers are G4S-type linkers (SEQ ID NO: 18), i.e., linkers containing one or more (e.g., 2, 3, or 4) GGGGS (SEQ ID NO: 15) motifs, or variations of the motif (such as ones that have one, two, or three amino acid insertions, deletions, and substitutions from the motif).

Linker design methods and illustrative linkers that may be used to link the ZFP domain and the transcription repressor domain of the present ZFP-TFs and/or the zinc fingers within the ZFP domains are described in U.S. Pat. Nos. 6,479,626; 7,851,216; 8,772,453; 9,394,531; 9,567,609; and 10,724,020; and PCT Publication Nos. WO 1999/045132; WO 2001/053480; WO 2009/154686; WO 2011/139349; WO 2015/031619; and WO 2017/136049. The proteins described herein may include any combination of suitable linkers.

Non-limiting examples of linkers are DGGGS (SEQ ID NO: 3), TGEKP (SEQ ID NO: 4), LRQKDGERP (SEQ ID NO: 5), GGRR (SEQ ID NO: 6), GGRRGGGS (SEQ ID NO: 7), LRQRDGERP (SEQ ID NO: 8), LRQKDGGGSERP (SEQ ID NO: 9), LRQKD(G₃S)₂ ERP (SEQ ID NO: 10), TGSQKP (SEQ ID NO: 11), LRQKDAARGS (SEQ ID NO: 13), LRQKDAARGSGG (SEQ ID NO: 14). Additional illustrative linkers for linking zinc fingers and/or for linking domains are listed in Table 1. The finger-finger linkers listed in Table 1 include portions of backbone sequence, e.g., FQ or FA.

Table 1 shows illustrative alternate peptide linkers that may be used to link zinc finger amino acid sequences and/or ZFP and functional domain sequences as shown in FIG. 14 or FIG. 16.

TABLE 1

| | | Exemplary Linker | | |
|---|---|---|---|---|
| Linker | Linker | Linker | | SEQ ID |
| Position | Category | Code | Peptide sequence | NO: |
| Finger-Finger | No base skipping | 0a | TGEKPFQ | 20 |
| | | 0b | TGGQRPFQ | 21 |
| | | 0c | TGSQKPFQ | 22 |
| | | 0d | TGSQRPFQ | 23 |
| | | 0f | TGEKPFA | 24 |
| | 1 base skipping | 1a | TGGGGSQRPFQ | 25 |
| | | 1b | TGGGGSQKPFQ | 26 |
| | | 1c | THPRAPIPKPFQ | 27 |
| | | 1d | TPNRRPAPKPFQ | 28 |
| | | 1e | TVPRPTPPKPFQ | 29 |
| | | 1f | TYPRPIAAKPFQ | 30 |
| | 2 base skipping | 2a | TGGGGSGGSQRPFQ | 31 |
| | | 2b | TGGGGSGGSQKPFQ | 32 |
| | | 2d | TLAPRPYRPPKPFQ | 33 |
| | | 2e | TPGGKSSRTDRNKPFQ | 34 |
| | | 2f | TPNPHRRTDPSHKPFQ | 35 |
| ZFP-functional | ZFP-TF | C0 | LRGSGG | 36 |
| domain | | C1 | LRQKDAARGS | 13 |
| (interdomain) | | C1k | LRQKDAARGSGG | 14 |

In some embodiments, the present engineered ZFPs described herein comprise two adjacent DNA-binding recognition helix sequences linked as shown in a single row of FIG. 14 or FIG. 16. For example, an engineered ZFP may comprise the sequences of F1-F2, F2-F3, F3-F4, F4-F5, or F5-F6 as shown in a single row of FIG. 14 or FIG. 16. In other embodiments, a different linker may be used from the same linker category. In some embodiments, the present engineered ZFPs described herein comprise the DNA-binding recognition helix sequences linked as shown in a single row of FIG. 14 or FIG. 16. For example, an engineered ZFP may comprise the linked sequences of F1-F4, F1-F5, or F1-F6 as shown in a single row of FIG. 14 or FIG. 16. In other embodiments, one or more different linkers may be used from the same linker category.

In some embodiments, an engineered ZFP-TF described herein comprises the recognition helix and linker portions of a sequence as shown in a single row of FIG. 14 or FIG. 16. In other embodiments, one or more different linkers may be used from the same linker category. In some embodiments, an engineered ZFP-TF described herein comprises the recognition helix, backbone, and linker portions of a sequence as shown in a single row of FIG. 15 or FIG. 17. In other embodiments, one or more different linkers may be used from the same linker category. In some embodiments, an engineered ZFP-TF described herein comprises an amino acid sequence as shown in a single row of FIG. 15 or FIG. 17. In some embodiments, an engineered ZFP-TF described herein comprises the recognition helix, backbone, and linker portions of a sequence shown in a single row of FIG. 15 or FIG. 17 as the sequence would appear following post-translational modification. In some embodiments, an engineered ZFP-TF described herein comprises an amino acid sequence as shown in a single row of FIG. 15 or FIG. 17 as the sequence would appear following post-translational modification. For example, post-translational modification may remove the initiator methionine residue from a sequence as shown in FIG. 15 or FIG. 17.

### IV. Expression of the ZFP-TFs

A ZFP-TF of the present disclosure may be introduced to a patient through a nucleic acid molecule encoding it. The nucleic acid molecule may be an RNA or cDNA molecule. The nucleic acid may be introduced into the brain of the patient through injection of a composition comprising a lipid:nucleic acid complex (e.g., a liposome). Alternatively, the ZFP-TF may be introduced to the patient through a nucleic acid expression vector comprising a sequence encoding the ZFP-TF. The expression vectors may include expression control sequences such as promoters, enhancers, transcription signal sequences, and transcription termination sequences that allow expression of the coding sequence for the ZFP-TFs in the cells of the nervous system. In some embodiments, the expression vector remains present in the cell as a stable episome. In other embodiments, the expression vector is integrated into the genome of the cell.

In some embodiments, the promoter on the vector for directing the ZFP-TF expression in the brain is a constitutive active promoter or an inducible promoter. Suitable promoters include, without limitation, a Rous sarcoma virus (RSV) long terminal repeat (LTR) promoter (optionally with an RSV enhancer), a cytomegalovirus (CMV) promoter (optionally with a CMV enhancer), a CMV immediate early promoter, a simian virus 40 (SV40) promoter, a dihydrofolate reductase (DHFR) promoter, a β-actin promoter, a phosphoglycerate kinase (PGK) promoter, an EFlα promoter, a Moloney murine leukemia virus (MoMLV) LTR, a creatine kinase-based (CK6) promoter, a transthyretin promoter (TTR), a thymidine kinase (TK) promoter, a tetracycline responsive promoter (TRE), a hepatitis B Virus (HBV) promoter, a human al-antitrypsin (hAAT) promoter, chimeric liver-specific promoters (LSPs), an E2 factor (E2F) promoter, the human telomerase reverse transcriptase (hTERT) promoter, a CMV enhancer/chicken β-actin/rabbit β-globin promoter (CAG promoter; Niwa et al., Gene (1991) 108(2):193-9), and an RU-486-responsive promoter. Neuron-specific promoters such as a synapsin I promoter, a calcium/calmodulin-dependent protein kinase II (CamKII) promoter, a methyl CpG-binding protein 2 (MeCP2) promoter, a choline acetyltransferase (ChAT) promoter, a Calbindin (Calb) promoter, a CAMKII promoter, a PrP promoter, a GFAP promoter, or an engineered or natural promoter that restricts expression to neuron and glial cells may also be used. Astrocyte-specific promoters such as the glial fibrillary acidic protein (GFAP) promoter or the aldehyde dehydrogenase 1 family, member L1 (Aldh1L1) promoter may also be used. Oligodendrocyte-specific promoters such as the Olig2 promoter may also be used. In addition, the promoter may include one or more self-regulating elements whereby the ZFP-TF can bind to and repress its own expression level to a preset threshold. See U.S. Pat. No. 9,624,498.

Any method of introducing the nucleotide sequence into a cell may be employed, including but not limited to, electroporation, calcium phosphate precipitation, microinjection, cationic or anionic liposomes, liposomes in combination with a nuclear localization signal, naturally occurring liposomes (e.g., exosomes), or viral transduction.

For in vivo delivery of an expression vector, viral transduction may be used. A variety of viral vectors known in the art may be adapted by one of skill in the art for use in the present disclosure, for example, vaccinia vectors, adenoviral vectors, lentiviral vectors, poxyviral vectors, adeno-associated viral (AAV) vectors, retroviral vectors, and hybrid viral vectors. In some embodiments, the viral vector used herein is a recombinant AAV (rAAV) vector. AAV vectors are especially suitable for CNS gene delivery because they infect both dividing and non-dividing cells, exist as stable episomal structures for long term expression, and have very low immunogenicity (Hadaczek et al., Mol Ther. (2010) 18:1458-61; Zaiss, et al., Gene Ther. (2008) 15:808-16). Any suitable AAV serotype may be used. For example, the AAV may be AAV1, AAV2, AAV3, AAV3B, AAV4, AAV5, AAV6, AAV7, AAV8, AAV8.2, AAV9, AAV.PHP.B, AAV.PHP.eB, or AAVrh10, or of a novel serotype or a pseudotype such as AAV2/8, AAV2/5, AAV2/6, AAV2/9, or AAV2/6/9, or a serotype that is the variant or derivative of one of the AAV serotypes listed herein (i.e., AAV derived from multiple serotypes; for example, the rAAV comprises AAV2 inverted terminal repeats (ITR) in its genome and an AAV8, 5, 6, or 9 capsid). In some embodiments, the expression vector is an AAV viral vector and is introduced to the target human cell by a recombinant AAV virion whose genome comprises the construct, including having the ITR sequences on both ends to allow the production of the AAV virion in a production system such as an insect cell/baculovirus production system or a mammalian cell production system. The AAV may be engineered such that its capsid proteins have reduced immunogenicity or enhanced transduction ability in humans or nonhuman primates. In some embodiments, AAV9 is used. Viral vectors described herein may be produced using methods known in the art. Any suitable permissive or packaging cell type may be employed to produce the viral particles. For example, mammalian (e.g., 293) or insect (e.g., sf9) cells may be used as the packaging cell line.

### V. Pharmaceutical Applications

The present ZFP-TFs can be used to treat patients in need of downregulation of tau expression. The patients suffer from, or are at risk of developing, neurodegenerative diseases such as Alzheimer's disease, frontotemporal dementia, progressive supranuclear palsy, traumatic brain injury, seizure disorders, corticobasal degeneration, Parkinson's disease, dementia with Lewy bodies (DLB) and/or any other tauopathies. Patients at risk include those who are genetically predisposed, those who have suffered repeated brain injuries such as concussions, and those who have been exposed to environmental neurotoxins. The present disclosure provides a method of treating a neurological disease (e.g., a tauopathy such as a neurodegenerative disease) in a subject such as a human patient in need thereof, comprising introducing to the nervous system of the subject a therapeutically effective amount (e.g., an amount that allows sufficient repression of MAPT expression) of the ZFP-TF (e.g., an rAAV vector expressing it). The term “treating” encompasses alleviation of symptoms, prevention of onset of symptoms, slowing of disease progression, improvement of quality of life, and increased survival.

The present disclosure provides a pharmaceutical composition comprising a viral vector such as an rAAV whose recombinant genome comprises an expression cassette for the ZFP-TFs. The pharmaceutical composition (e.g., an artificial cerebrospinal fluid or aCSF), may further comprise a pharmaceutically acceptable carrier such as water, saline (e.g., phosphate-buffered saline), dextrose, glycerol, sucrose, lactose, gelatin, dextran, albumin, or pectin. In addition, the composition may contain auxiliary substances, such as, wetting or emulsifying agents, pH-buffering agents, stabilizing agents, or other reagents that enhance the effectiveness of the pharmaceutical composition. The pharmaceutical composition may contain delivery vehicles such as liposomes, nanocapsules, microparticles, microspheres, lipid particles, and vesicles.

The cells targeted by the therapeutics of the present disclosure are cells in the brain, including, without limitation, a neuronal cell (e.g., a motor neuron, a sensory neuron, a dopaminergic neuron, a cholinergic neuron, a glutamatergic neuron, a GABAergic neuron, or a serotonergic neuron); a glial cell (e.g., an oligodendrocyte, an astrocyte, a pericyte, a Schwann cell, or a microglial cell); an ependymal cell; or a neuroepithelial cell.

The brain regions targeted by the therapeutics may be those most significantly affected in tauopathies, such as certain cortical regions, the entorhinal cortex, the hippocampus, the cerebellum, the globus pallidus, the thalamus, the midbrain, the caudate, the putamen, the substantia nigra, the pons, and the medulla. These regions can be reached directly through intrahippocampal injection, intracerebral injection, intra-cisterna magna (ICM) injection, or more generally through intraparenchymal injection, intrathalamic injection, intracerebroventricular (ICV) injection, intrathecal injection, or intravenous injection. Other routes of administration include, without limitation, intracerebral, intraventricular, intranasal, or intraocular administration. In some embodiments, the viral vector spreads throughout the CNS tissue following direct administration into the cerebrospinal fluid (CSF), e.g., via intrathecal and/or intracerebral injection, or intra-cisterna magna injection or intracerebroventricular injection. In other embodiments, the viral vectors cross the blood-brain barrier and achieve wide-spread distribution throughout the CNS tissue of a subject following intravenous administration. In other embodiments, the viral vectors are delivered directly to the target regions via intraparenchymal injections. In some cases, the viral vectors may undergo retrograde or anterograde transport to other brain regions following intraparenchymal delivery. In some aspects, the viral vectors have distinct CNS tissue targeting capabilities (e.g., CNS tissue tropisms), which achieve stable and nontoxic gene transfer at high efficiencies.

By way of example, the pharmaceutical composition may be provided to the patient through intraventricular administration, e.g., into a ventricular region of the forebrain of the patient such as the right lateral ventricle, the left lateral ventricle, the third ventricle, or the fourth ventricle. The pharmaceutical composition may be provided to the patient through intracerebral administration, e.g., injection of the composition into or near the cerebrum, medulla, pons, cerebellum, thalamus, striatum, caudate, putamen, substantia nigra, midbrain, caudate, putamen, olfactory bulb, locus coeruleus, brain stem, globus pallidus, hippocampus, cerebral cortex, intracranial cavity, meninges, dura mater, arachnoid mater, or pia mater of the brain. Intracerebral administration may include, in some cases, administration of an agent into the cerebrospinal fluid (CSF) of the subarachnoid space surrounding the brain.

In some cases, intracerebral administration involves injection using stereotaxic procedures. Stereotaxic procedures are well known in the art and typically involve the use of a computer and a 3-dimensional scanning device that are used together to guide injection to a particular intracerebral region, e.g., a ventricular region. Micro-injection pumps (e.g., from World Precision Instruments) may also be used. In some cases, a microinjection pump is used to deliver a composition comprising a viral vector. In some cases, the infusion rate of the composition is in a range of 0.1 μl/min to 100 μl/min. As will be appreciated by the skilled artisan, infusion rates will depend on a variety of factors, including, for example, species of the subject, age of the subject, weight/size of the subject, serotype of the AAV, dosage required, and intracerebral region targeted. Thus, other infusion rates may be deemed by a skilled artisan to be appropriate in certain circumstances.

Delivery of rAAVs to a subject may be accomplished, for example, by intravenous administration. In certain instances, it may be desirable to deliver the rAAVs (e.g., 10¹⁰-10¹⁵ Vg) locally to the brain tissue, the spinal cord, cerebrospinal fluid (CSF), neuronal cells, glial cells, meninges, astrocytes, oligodendrocytes, microglia, interstitial spaces, and the like. In some cases, recombinant AAVs may be delivered directly to the CNS by injection into or near the ventricular region, as well as to the hippocampus, cerebral cortex, cerebellar lobule, cerebellum, cerebrum, medulla, pons, thalamus, striatum, caudate, putamen, substantia nigra, midbrain, caudate, putamen, olfactory bulb, locus coeruleus, brain stem, globus pallidus, intracranial cavity, meninges, dura mater, arachnoid mater, or pia mater of the brain, or other brain region. AAVs may be delivered with a needle, catheter or related device, using neurosurgical techniques known in the art, such as by stereotactic injection (see, e.g., Stein et al., J Vir. (1999) 73:3424-9; Davidson et al., PNAS (2000) 97:3428-32; Davidson et al., Nat Genet. (1993) 3:219-223; and Alisky and Davidson, Hum Gene Ther. (2000) 11:2315-29).

Unless otherwise defined herein, scientific and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control. Generally, nomenclature used in connection with, and techniques of neurology, medicine, medicinal and pharmaceutical chemistry, and cell biology described herein are those well-known and commonly used in the art. Enzymatic reactions and purification techniques are performed according to manufacturer's specifications, as commonly accomplished in the art or as described herein. Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. All publications and other references mentioned herein are incorporated by reference in their entirety. Although a number of documents are cited herein, this citation does not constitute an admission that any of these documents forms part of the common general knowledge in the art. As used herein, the term “approximately” or “about” as applied to one or more values of interest refers to a value that is similar to a stated reference value. In certain embodiments, the term refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context.

In order that this invention may be better understood, the following examples are set forth. These examples are for purposes of illustration only and are not to be construed as limiting the scope of the invention in any manner.

### VI. Exemplary Embodiments

Non-limiting exemplary embodiments of the present disclosure are described below.

1. A ZFP-TF fusion protein that binds to a target sequence and comprises the DNA-binding zinc finger recognition helix sequences corresponding to a ZFP ID as shown in a single row of FIG. 14, wherein the ZFP ID is 52288.
2. A ZFP-TF fusion protein that binds to a target sequence and comprises the DNA-binding zinc finger recognition helix sequences corresponding to a ZFP ID as shown in a single row of FIG. 14, wherein the ZFP ID is 52389.
3. A ZFP-TF fusion protein that binds to a target sequence and comprises the DNA-binding zinc finger recognition helix sequences corresponding to a ZFP ID as shown in a single row of FIG. 14, wherein the ZFP ID is 52364.
4. A ZFP-TF fusion protein that binds to a target sequence and comprises the DNA-binding zinc finger recognition helix sequences corresponding to a ZFP ID as shown in a single row of FIG. 14, wherein the ZFP ID is 57890.
5. A ZFP-TF fusion protein that binds to a target sequence and comprises the DNA-binding zinc finger recognition helix sequences corresponding to a ZFP ID as shown in a single row of FIG. 14, wherein the ZFP ID is 71214.
6. A ZFP-TF fusion protein that binds to a target sequence and comprises the DNA-binding zinc finger recognition helix sequences corresponding to a ZFP ID as shown in a single row of FIG. 14, wherein the ZFP ID is 71218.
7. A ZFP-TF fusion protein that binds to a target sequence and comprises the DNA-binding zinc finger recognition helix sequences corresponding to a ZFP ID as shown in a single row of FIG. 14, wherein the ZFP ID is 71225.
8. A ZFP-TF fusion protein that binds to a target sequence and comprises the DNA-binding zinc finger recognition helix sequences corresponding to a ZFP ID as shown in a single row of FIG. 14, wherein the ZFP ID is 71227.
9. A ZFP-TF fusion protein that binds to a target sequence and comprises the DNA-binding zinc finger recognition helix sequences corresponding to a ZFP ID as shown in a single row of FIG. 14, wherein the ZFP ID is 71249.
10. A ZFP-TF fusion protein that binds to a target sequence and comprises the DNA-binding zinc finger recognition helix sequences corresponding to a ZFP ID as shown in a single row of FIG. 14, wherein the ZFP ID is 71304.
11. A ZFP-TF fusion protein that binds to a target sequence and comprises the DNA-binding zinc finger recognition helix sequences corresponding to a ZFP ID as shown in a single row of FIG. 14, wherein the ZFP ID is 71309.
12. A ZFP-TF fusion protein that binds to a target sequence and comprises the DNA-binding zinc finger recognition helix sequences corresponding to a ZFP ID as shown in a single row of FIG. 14, wherein the ZFP ID is 71310.
13. A ZFP-TF fusion protein that binds to a target sequence and comprises the DNA-binding zinc finger recognition helix sequences corresponding to a ZFP ID as shown in a single row of FIG. 14, wherein the ZFP ID is 71312.
14. A ZFP-TF fusion protein that binds to a target sequence and comprises the DNA-binding zinc finger recognition helix sequences corresponding to a ZFP ID as shown in a single row of FIG. 14, wherein the ZFP ID is 71341.
15. A ZFP-TF fusion protein that binds to a target sequence and comprises the DNA-binding zinc finger recognition helix sequences corresponding to a ZFP ID as shown in a single row of FIG. 14, wherein the ZFP ID is 71343.
16. A ZFP-TF fusion protein that binds to a target sequence and comprises the DNA-binding zinc finger recognition helix sequences corresponding to a ZFP ID as shown in a single row of FIG. 14, wherein the ZFP ID is 71345.
17. A ZFP-TF fusion protein that binds to a target sequence and comprises the DNA-binding zinc finger recognition helix sequences corresponding to a ZFP ID as shown in a single row of FIG. 14, wherein the ZFP ID is 71347.
18. A ZFP-TF fusion protein that binds to a target sequence and comprises the DNA-binding zinc finger recognition helix sequences corresponding to a ZFP ID as shown in a single row of FIG. 14, wherein the ZFP ID is 71351.
19. A ZFP-TF fusion protein that binds to a target sequence and comprises the DNA-binding zinc finger recognition helix sequences corresponding to a ZFP ID as shown in a single row of FIG. 14, wherein the ZFP ID is 71352.
20. A ZFP-TF fusion protein that binds to a target sequence and comprises the DNA-binding zinc finger recognition helix sequences corresponding to a ZFP ID as shown in a single row of FIG. 14, wherein the ZFP ID is 71357.
21. A ZFP-TF fusion protein that binds to a target sequence and comprises the DNA-binding zinc finger recognition helix sequences corresponding to a ZFP ID as shown in a single row of FIG. 14, wherein the ZFP ID is 71364.
22. A ZFP-TF fusion protein that binds to a target sequence and comprises the DNA-binding zinc finger recognition helix sequences corresponding to a ZFP ID as shown in a single row of FIG. 14, wherein the ZFP ID is 71366.
23. A ZFP-TF fusion protein that binds to a target sequence and comprises the DNA-binding zinc finger recognition helix sequences corresponding to a ZFP ID as shown in a single row of FIG. 14, wherein the ZFP ID is 71370.
24. A ZFP-TF fusion protein that binds to a target sequence and comprises the DNA-binding zinc finger recognition helix sequences corresponding to a ZFP ID as shown in a single row of FIG. 14, wherein the ZFP ID is 71373.
25. A ZFP-TF fusion protein that binds to a target sequence and comprises the DNA-binding zinc finger recognition helix sequences corresponding to a ZFP ID as shown in a single row of FIG. 14, wherein the ZFP ID is 71374.
26. A ZFP-TF fusion protein that binds to a target sequence and comprises the DNA-binding zinc finger recognition helix sequences corresponding to a ZFP ID as shown in a single row of FIG. 14, wherein the ZFP ID is 71377.
27. A ZFP-TF fusion protein that binds to a target sequence and comprises the DNA-binding zinc finger recognition helix sequences corresponding to a ZFP ID as shown in a single row of FIG. 14, wherein the ZFP ID is 71378.
28. A ZFP-TF fusion protein that binds to a target sequence and comprises the DNA-binding zinc finger recognition helix sequences corresponding to a ZFP ID as shown in a single row of FIG. 14, wherein the ZFP ID is 71385.
29. A ZFP-TF fusion protein that binds to a target sequence and comprises the DNA-binding zinc finger recognition helix sequences corresponding to a ZFP ID as shown in a single row of FIG. 14, wherein the ZFP ID is 71389.
30. A ZFP-TF fusion protein that binds to a target sequence and comprises the DNA-binding zinc finger recognition helix sequences corresponding to a ZFP ID as shown in a single row of FIG. 14, wherein the ZFP ID is 71391.
31. A ZFP-TF fusion protein that binds to a target sequence and comprises the DNA-binding zinc finger recognition helix sequences corresponding to a ZFP ID as shown in a single row of FIG. 14, wherein the ZFP ID is 71393.
32. A ZFP-TF fusion protein that binds to a target sequence and comprises the DNA-binding zinc finger recognition helix sequences corresponding to a ZFP ID as shown in a single row of FIG. 14, wherein the ZFP ID is 71395.
33. A ZFP-TF fusion protein that binds to a target sequence and comprises the DNA-binding zinc finger recognition helix sequences corresponding to a ZFP ID as shown in a single row of FIG. 14, wherein the ZFP ID is 71397.
34. A ZFP-TF fusion protein that binds to a target sequence and comprises the DNA-binding zinc finger recognition helix sequences corresponding to a ZFP ID as shown in a single row of FIG. 14, wherein the ZFP ID is 71398.
35. A ZFP-TF fusion protein that binds to a target sequence and comprises the DNA-binding zinc finger recognition helix sequences corresponding to a ZFP ID as shown in a single row of FIG. 14, wherein the ZFP ID is 71399.
36. A ZFP-TF fusion protein that binds to a target sequence and comprises the DNA-binding zinc finger recognition helix sequences corresponding to a ZFP ID as shown in a single row of FIG. 14, wherein the ZFP ID is 71400.
37. A ZFP-TF fusion protein that binds to a target sequence and comprises the DNA-binding zinc finger recognition helix sequences corresponding to a ZFP ID as shown in a single row of FIG. 14, wherein the ZFP ID is 71401.
38. A ZFP-TF fusion protein that binds to a target sequence and comprises the DNA-binding zinc finger recognition helix sequences corresponding to a ZFP ID as shown in a single row of FIG. 14, wherein the ZFP ID is 71402.
39. A ZFP-TF fusion protein that binds to a target sequence and comprises the DNA-binding zinc finger recognition helix sequences corresponding to a ZFP ID as shown in a single row of FIG. 14, wherein the ZFP ID is 71414.
40. A ZFP-TF fusion protein that binds to a target sequence and comprises the DNA-binding zinc finger recognition helix sequences corresponding to a ZFP ID as shown in a single row of FIG. 14, wherein the ZFP ID is 71420.
41. A ZFP-TF fusion protein that binds to a target sequence and comprises the DNA-binding zinc finger recognition helix sequences corresponding to a ZFP ID as shown in a single row of FIG. 14, wherein the ZFP ID is 71421.
42. A ZFP-TF fusion protein that binds to a target sequence and comprises the DNA-binding zinc finger recognition helix sequences corresponding to a ZFP ID as shown in a single row of FIG. 14, wherein the ZFP ID is 71424.
43. A ZFP-TF fusion protein that binds to a target sequence and comprises the DNA-binding zinc finger recognition helix sequences corresponding to a ZFP ID as shown in a single row of FIG. 14, wherein the ZFP ID is 71437.
44. A ZFP-TF fusion protein that binds to a target sequence and comprises the DNA-binding zinc finger recognition helix sequences corresponding to a ZFP ID as shown in a single row of FIG. 14, wherein the ZFP ID is 71447.
45. A ZFP-TF fusion protein that binds to a target sequence and comprises the DNA-binding zinc finger recognition helix sequences corresponding to a ZFP ID as shown in a single row of FIG. 14, wherein the ZFP ID is 71448.
46. A ZFP-TF fusion protein that binds to a target sequence and comprises the DNA-binding zinc finger recognition helix sequences corresponding to a ZFP ID as shown in a single row of FIG. 14, wherein the ZFP ID is 71453.
47. A ZFP-TF fusion protein that binds to a target sequence and comprises the DNA-binding zinc finger recognition helix sequences corresponding to a ZFP ID as shown in a single row of FIG. 14, wherein the ZFP ID is 71467.
48. A ZFP-TF fusion protein that binds to a target sequence and comprises the DNA-binding zinc finger recognition helix sequences corresponding to a ZFP ID as shown in a single row of FIG. 14, wherein the ZFP ID is 71468.
49. A ZFP-TF fusion protein that binds to a target sequence and comprises the DNA-binding zinc finger recognition helix sequences corresponding to a ZFP ID as shown in a single row of FIG. 14, wherein the ZFP ID is 71470.
50. A ZFP-TF fusion protein that binds to a target sequence and comprises the DNA-binding zinc finger recognition helix sequences corresponding to a ZFP ID as shown in a single row of FIG. 14, wherein the ZFP ID is 71472.
51. A ZFP-TF fusion protein that binds to a target sequence and comprises the DNA-binding zinc finger recognition helix sequences corresponding to a ZFP ID as shown in a single row of FIG. 14, wherein the ZFP ID is 71485.
52. A ZFP-TF fusion protein that binds to a target sequence and comprises the DNA-binding zinc finger recognition helix sequences corresponding to a ZFP ID as shown in a single row of FIG. 14, wherein the ZFP ID is 71503.
53. A ZFP-TF fusion protein that binds to a target sequence and comprises the DNA-binding zinc finger recognition helix sequences and backbone mutation(s) corresponding to a ZFP ID as shown in a single row of FIG. 16, wherein the ZFP ID is 65918.
54. A ZFP-TF fusion protein that binds to a target sequence and comprises the DNA-binding zinc finger recognition helix sequences and backbone mutation(s) corresponding to a ZFP ID as shown in a single row of FIG. 16, wherein the ZFP ID is 73015.
55. A ZFP-TF fusion protein that binds to a target sequence and comprises the DNA-binding zinc finger recognition helix sequences and backbone mutation(s) corresponding to a ZFP ID as shown in a single row of FIG. 16, wherein the ZFP ID is 73016.
56. A ZFP-TF fusion protein that binds to a target sequence and comprises the DNA-binding zinc finger recognition helix sequences and backbone mutation(s) corresponding to a ZFP ID as shown in a single row of FIG. 16, wherein the ZFP ID is 73017.
57. A ZFP-TF fusion protein that binds to a target sequence and comprises the DNA-binding zinc finger recognition helix sequences and backbone mutation(s) corresponding to a ZFP ID as shown in a single row of FIG. 16, wherein the ZFP ID is 73018.
58. A ZFP-TF fusion protein that binds to a target sequence and comprises the DNA-binding zinc finger recognition helix sequences and backbone mutation(s) corresponding to a ZFP ID as shown in a single row of FIG. 16, wherein the ZFP ID is 73019.
59. A ZFP-TF fusion protein that binds to a target sequence and comprises the DNA-binding zinc finger recognition helix sequences and backbone mutation(s) corresponding to a ZFP ID as shown in a single row of FIG. 16, wherein the ZFP ID is 73020.
60. A ZFP-TF fusion protein that binds to a target sequence and comprises the DNA-binding zinc finger recognition helix sequences and backbone mutation(s) corresponding to a ZFP ID as shown in a single row of FIG. 16, wherein the ZFP ID is 73021.
61. A ZFP-TF fusion protein that binds to a target sequence and comprises the DNA-binding zinc finger recognition helix sequences and backbone mutation(s) corresponding to a ZFP ID as shown in a single row of FIG. 16, wherein the ZFP ID is 73029.
62. A ZFP-TF fusion protein that binds to a target sequence and comprises the DNA-binding zinc finger recognition helix sequences and backbone mutation(s) corresponding to a ZFP ID as shown in a single row of FIG. 16, wherein the ZFP ID is 73030.
63. A ZFP-TF fusion protein that binds to a target sequence and comprises the DNA-binding zinc finger recognition helix sequences and backbone mutation(s) corresponding to a ZFP ID as shown in a single row of FIG. 16, wherein the ZFP ID is 73031.
64. A ZFP-TF fusion protein that binds to a target sequence and comprises the DNA-binding zinc finger recognition helix sequences and backbone mutation(s) corresponding to a ZFP ID as shown in a single row of FIG. 16, wherein the ZFP ID is 73032.
65. A ZFP-TF fusion protein that binds to a target sequence and comprises the DNA-binding zinc finger recognition helix sequences and backbone mutation(s) corresponding to a ZFP ID as shown in a single row of FIG. 16, wherein the ZFP ID is 73034.
66. A ZFP-TF fusion protein that binds to a target sequence and comprises the DNA-binding zinc finger recognition helix sequences and backbone mutation(s) corresponding to a ZFP ID as shown in a single row of FIG. 16, wherein the ZFP ID is 73035.
67. A ZFP-TF fusion protein that binds to a target sequence and comprises the DNA-binding zinc finger recognition helix sequences and backbone mutation(s) corresponding to a ZFP ID as shown in a single row of FIG. 16, wherein the ZFP ID is 73120.
68. A ZFP-TF fusion protein that binds to a target sequence and comprises the DNA-binding zinc finger recognition helix sequences and backbone mutation(s) corresponding to a ZFP ID as shown in a single row of FIG. 16, wherein the ZFP ID is 73121.
69. A ZFP-TF fusion protein that binds to a target sequence and comprises the DNA-binding zinc finger recognition helix sequences and backbone mutation(s) corresponding to a ZFP ID as shown in a single row of FIG. 16, wherein the ZFP ID is 73122.
70. A ZFP-TF fusion protein that binds to a target sequence and comprises the DNA-binding zinc finger recognition helix sequences and backbone mutation(s) corresponding to a ZFP ID as shown in a single row of FIG. 16, wherein the ZFP ID is 73123.
71. A ZFP-TF fusion protein that binds to a target sequence and comprises the DNA-binding zinc finger recognition helix sequences and backbone mutation(s) corresponding to a ZFP ID as shown in a single row of FIG. 16, wherein the ZFP ID is 73124.
72. A ZFP-TF fusion protein that binds to a target sequence and comprises the DNA-binding zinc finger recognition helix sequences and backbone mutation(s) corresponding to a ZFP ID as shown in a single row of FIG. 16, wherein the ZFP ID is 73125.
73. A ZFP-TF fusion protein that binds to a target sequence and comprises the DNA-binding zinc finger recognition helix sequences and backbone mutation(s) corresponding to a ZFP ID as shown in a single row of FIG. 16, wherein the ZFP ID is 73126.
74. A ZFP-TF fusion protein that binds to a target sequence and comprises the DNA-binding zinc finger recognition helix sequences and backbone mutation(s) corresponding to a ZFP ID as shown in a single row of FIG. 16, wherein the ZFP ID is 73127.
75. A ZFP-TF fusion protein that binds to a target sequence and comprises the DNA-binding zinc finger recognition helix sequences and backbone mutation(s) corresponding to a ZFP ID as shown in a single row of FIG. 16, wherein the ZFP ID is 73128.
76. A ZFP-TF fusion protein that binds to a target sequence and comprises the DNA-binding zinc finger recognition helix sequences and backbone mutation(s) corresponding to a ZFP ID as shown in a single row of FIG. 16, wherein the ZFP ID is 73129.
77. A ZFP-TF fusion protein that binds to a target sequence and comprises the DNA-binding zinc finger recognition helix sequences and backbone mutation(s) corresponding to a ZFP ID as shown in a single row of FIG. 16, wherein the ZFP ID is 73130.
78. A ZFP-TF fusion protein that binds to a target sequence and comprises the DNA-binding zinc finger recognition helix sequences and backbone mutation(s) corresponding to a ZFP ID as shown in a single row of FIG. 16, wherein the ZFP ID is 73131.
79. A ZFP-TF fusion protein that binds to a target sequence and comprises the DNA-binding zinc finger recognition helix sequences and backbone mutation(s) corresponding to a ZFP ID as shown in a single row of FIG. 16, wherein the ZFP ID is 73133.
80. A ZFP-TF fusion protein that binds to a target sequence and comprises the DNA-binding zinc finger recognition helix sequences and backbone mutation(s) corresponding to a ZFP ID as shown in a single row of FIG. 16, wherein the ZFP ID is 73190.
81. A ZFP-TF fusion protein that binds to a target sequence and comprises the DNA-binding zinc finger recognition helix sequences and backbone mutation(s) corresponding to a ZFP ID as shown in a single row of FIG. 16, wherein the ZFP ID is 73191.
82. A ZFP-TF fusion protein that binds to a target sequence and comprises the DNA-binding zinc finger recognition helix sequences and backbone mutation(s) corresponding to a ZFP ID as shown in a single row of FIG. 16, wherein the ZFP ID is 73192.
83. A ZFP-TF fusion protein that binds to a target sequence and comprises the DNA-binding zinc finger recognition helix sequences and backbone mutation(s) corresponding to a ZFP ID as shown in a single row of FIG. 16, wherein the ZFP ID is 73193.
84. A ZFP-TF fusion protein that binds to a target sequence and comprises the DNA-binding zinc finger recognition helix sequences and backbone mutation(s) corresponding to a ZFP ID as shown in a single row of FIG. 16, wherein the ZFP ID is 73194.
85. A ZFP-TF fusion protein that binds to a target sequence and comprises the DNA-binding zinc finger recognition helix sequences and backbone mutation(s) corresponding to a ZFP ID as shown in a single row of FIG. 16, wherein the ZFP ID is 73195.
86. A ZFP-TF fusion protein that binds to a target sequence and comprises the DNA-binding zinc finger recognition helix sequences and backbone mutation(s) corresponding to a ZFP ID as shown in a single row of FIG. 16, wherein the ZFP ID is 73196.
87. A ZFP-TF fusion protein that binds to a target sequence and comprises the DNA-binding zinc finger recognition helix sequences and backbone mutation(s) corresponding to a ZFP ID as shown in a single row of FIG. 16, wherein the ZFP ID is 73197.
88. A ZFP-TF fusion protein that binds to a target sequence and comprises the DNA-binding zinc finger recognition helix sequences and backbone mutation(s) corresponding to a ZFP ID as shown in a single row of FIG. 16, wherein the ZFP ID is 73198.
89. A ZFP-TF fusion protein that binds to a target sequence and comprises the DNA-binding zinc finger recognition helix sequences and backbone mutation(s) corresponding to a ZFP ID as shown in a single row of FIG. 16, wherein the ZFP ID is 73199.
90. A ZFP-TF fusion protein that binds to a target sequence and comprises the DNA-binding zinc finger recognition helix sequences and backbone mutation(s) corresponding to a ZFP ID as shown in a single row of FIG. 16, wherein the ZFP ID is 73200.
91. A ZFP-TF fusion protein that binds to a target sequence and comprises the DNA-binding zinc finger recognition helix sequences and backbone mutation(s) corresponding to a ZFP ID as shown in a single row of FIG. 16, wherein the ZFP ID is 73201.
92. A ZFP-TF fusion protein that binds to a target sequence and comprises the DNA-binding zinc finger recognition helix sequences and backbone mutation(s) corresponding to a ZFP ID as shown in a single row of FIG. 16, wherein the ZFP ID is 73202.
93. A ZFP-TF fusion protein that binds to a target sequence and comprises the DNA-binding zinc finger recognition helix sequences and backbone mutation(s) corresponding to a ZFP ID as shown in a single row of FIG. 16, wherein the ZFP ID is 73203.
94. The ZFP-TF fusion protein of any one of embodiments 1-93, wherein the ZFP-TF fusion protein comprises a transcription repressor domain.
95. The ZFP-TF fusion protein of embodiment 94, wherein transcription repressor domain comprises a KRAB domain.
96. The ZFP-TF fusion protein of embodiment 94, wherein transcription repressor domain comprises SEQ ID NO: 2.
97. A method of inhibiting expression of tau in a human brain cell, comprising introducing into the cell a fusion protein of any one of embodiments 1-96.
98. A method of inhibiting expression of tau in a human brain cell, comprising introducing into the cell two or more different fusion proteins according to any one of embodiments 1-96.
99. A method of inhibiting expression of tau in a human brain cell, comprising introducing into the cell the fusion proteins according to embodiment 4 (ZFP ID 57890) and embodiment 53 (ZFP ID 65918).
100. The method of embodiment 98 or 99, wherein each of the fusion proteins comprise a transcription repressor domain, optionally wherein the transcription repressor domain is a KRAB domain.
101. The method of any one of embodiments 98-100, wherein the fusion proteins are co-delivered.
102. The method of any one of embodiments 98-100, wherein the fusion proteins are linked by a 2A self-cleaving peptide.

### EXAMPLES

### Example 1: Screening of Anti-Tau ZFP-TFs

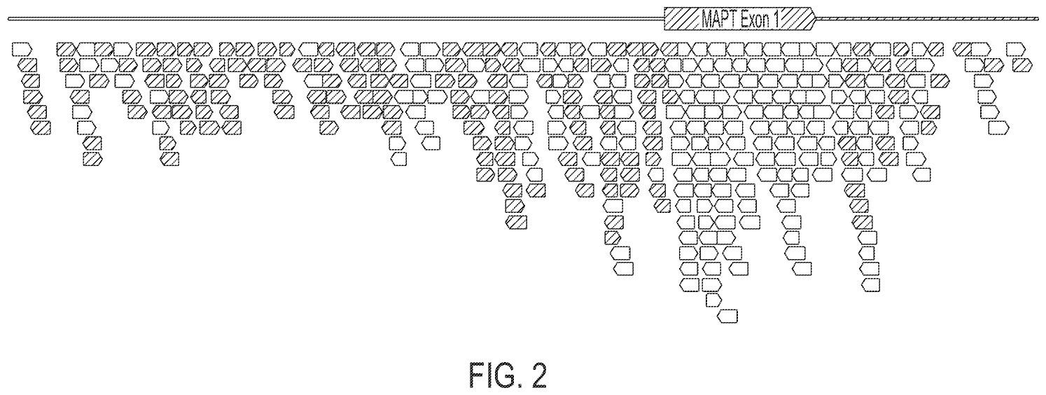

In order to identify ZFP-TFs that repress the expression of tau, a library of 370 ZFP-TFs predicted to bind 15, 18, or 19 bp sequences in the region of the human MAPT gene spanning from 1000 bp upstream to 500 bp downstream of the TSS was designed and screened for tau repression activity. The target regions of the ZFP-TFs are denoted by pentagons in FIG. 2, with the direction of the pentagon indicating the strand of DNA the ZFP-TF binds to (5′ to 3′). In this study, a KRAB domain sequence (SEQ ID NO: 2) was used as the transcription repressor and fused to the C-terminus of the ZFP domain. The sequences of 52 representative ZFP-TFs are shown in FIG. 14 below. Templates for in vitro transcription were generated from pVAX-ZFP or pVAX-GFP plasmids using PCR (forward primer GCAGAGCTCTCTGGCTAACTAGAG (SEQ ID NO: 16); reverse primer T(180)CTGGCAACTAGAAGGCACAG (SEQ ID NO: 17). Messenger RNA was synthesized using an mMESSAGE mMACHINE T7 ULTRA Transcription Kit (Thermo Fisher Scientific) as per the manufacturer's instructions and purified using RNeasy96 columns (Qiagen). mRNA encoding each ZFP-TF was then aliquoted into 96-well plates in a 6-dose dilution.

The screening was performed in the SK-N-MC human neuroepithelial cell line. SK-N-MC cells express human tau at high levels and are thus appropriate for testing of ZFP-TFs that reduce tau expression. The SK-N-MC cells were cultured in tissue culture flasks until confluency. The cells were plated on 96-well plates at 150,000 cells per well and were resuspended in Amaxa® SF solution. The cells were then mixed with ZFP-TF mRNA (6 doses: 3, 10, 30, 100, 300, and 1000 ng) and transferred to Amaxa® shuttle plate wells. The cells were transfected using the Amaxa® Nucleofector® device (Lonza; program CM-137). Eagle's MEM cell media was added to each well of the plate. The cells were transferred to a 96-well tissue culture plate and incubated at 37° C. for 20 hours.

The cells were then lysed and reverse transcription was performed using the C2CT kit following the manufacturer's instructions. TaqMan quantitative polymerase chain reaction (qPCR) was used to measure the expression levels of MAPT, which were normalized to the geometric mean of the expression levels of the housekeeping genes Atp5b and Eif4a2. A mock transfection and transfection with a ZFP-TF known not to target MAPT were used as negative controls.

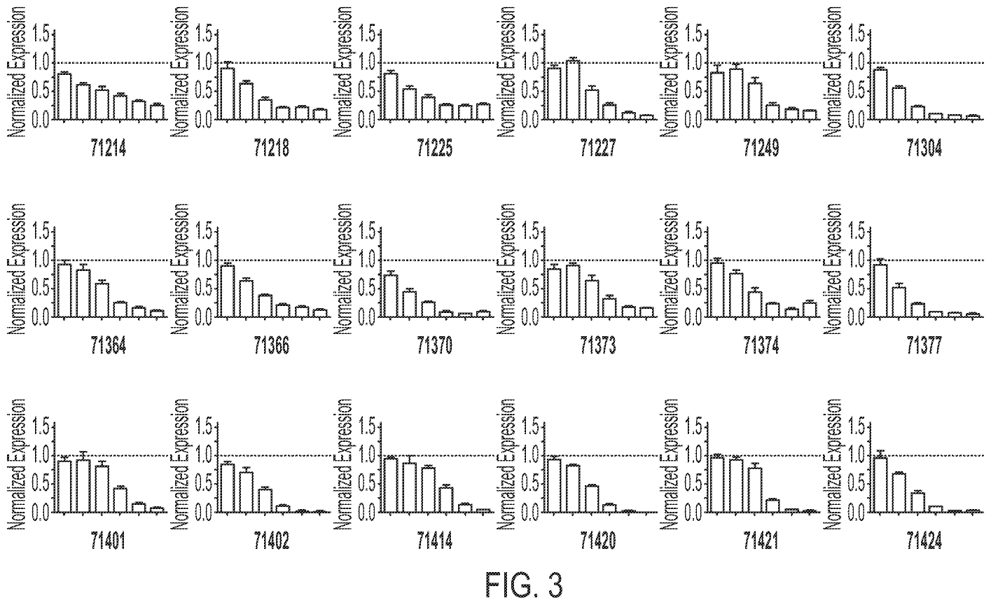

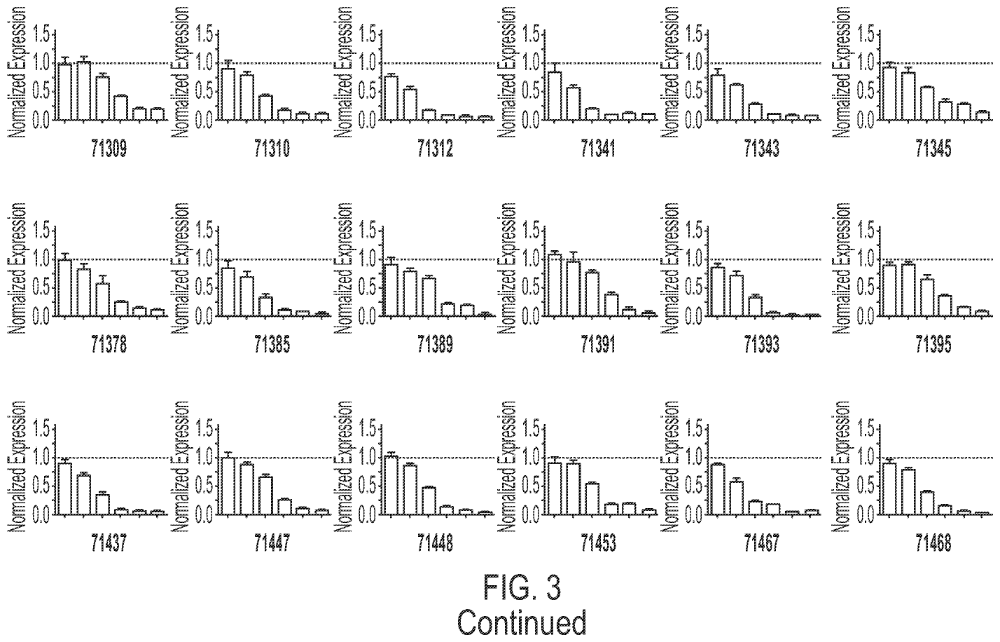

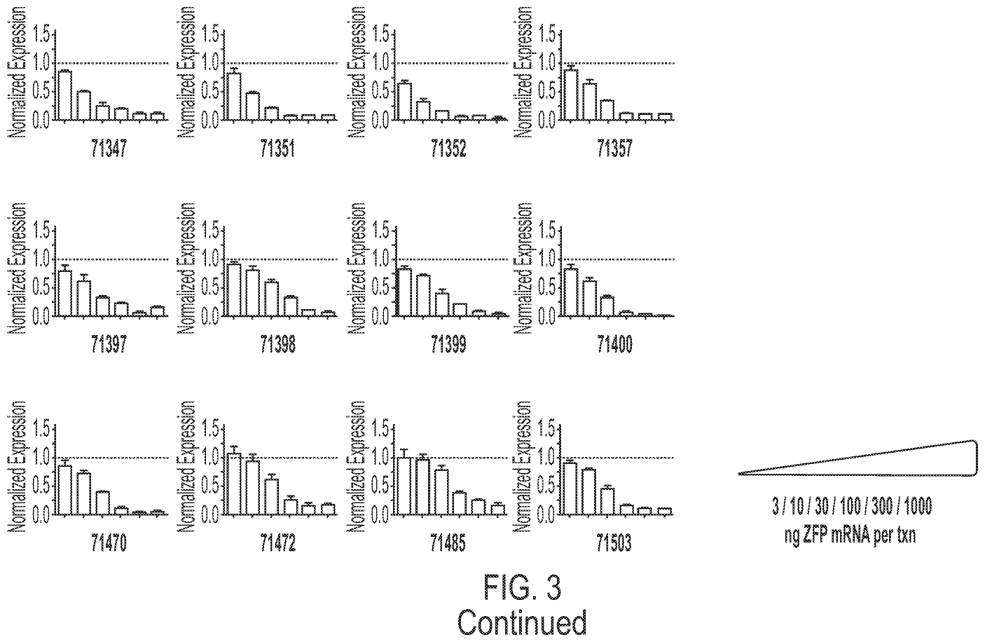

We found ≥50% dose-dependent repression of tau with ˜29% of the ZFP-TFs tested. The maximum repression achieved was 100%, but we also identified ZFP-TFs that repressed tau to a lesser degree (e.g., about 90%, about 75%, or about 50% at the highest dose). FIGS. 3A-D show the screening data. We also tested 52 representative ZFP-TFs in human iPSC-derived neurons (data not shown).

### Example 2: Optimization of Target Specificity of Anti-Tau ZFP-TFs

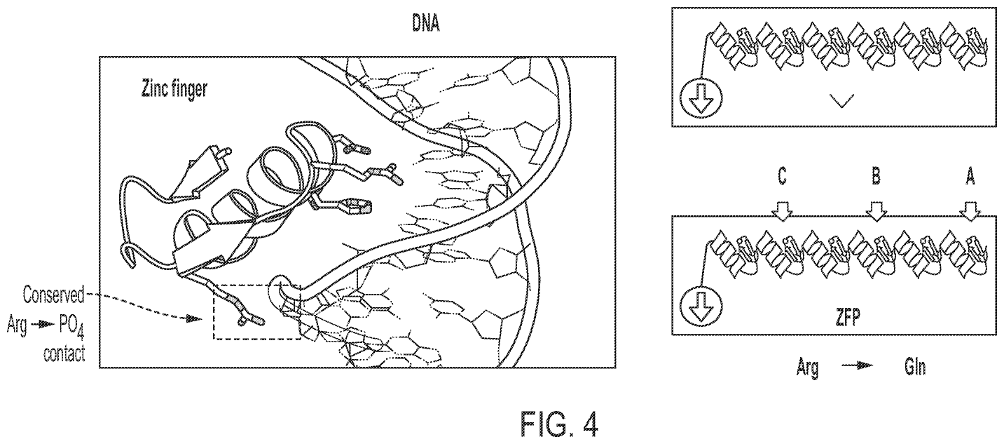

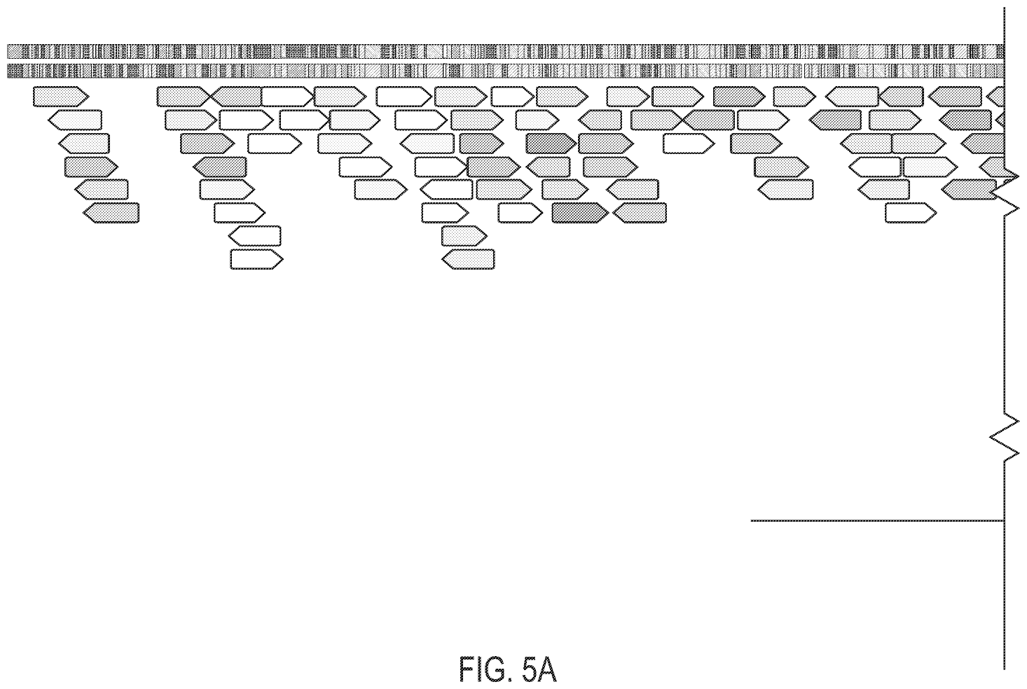

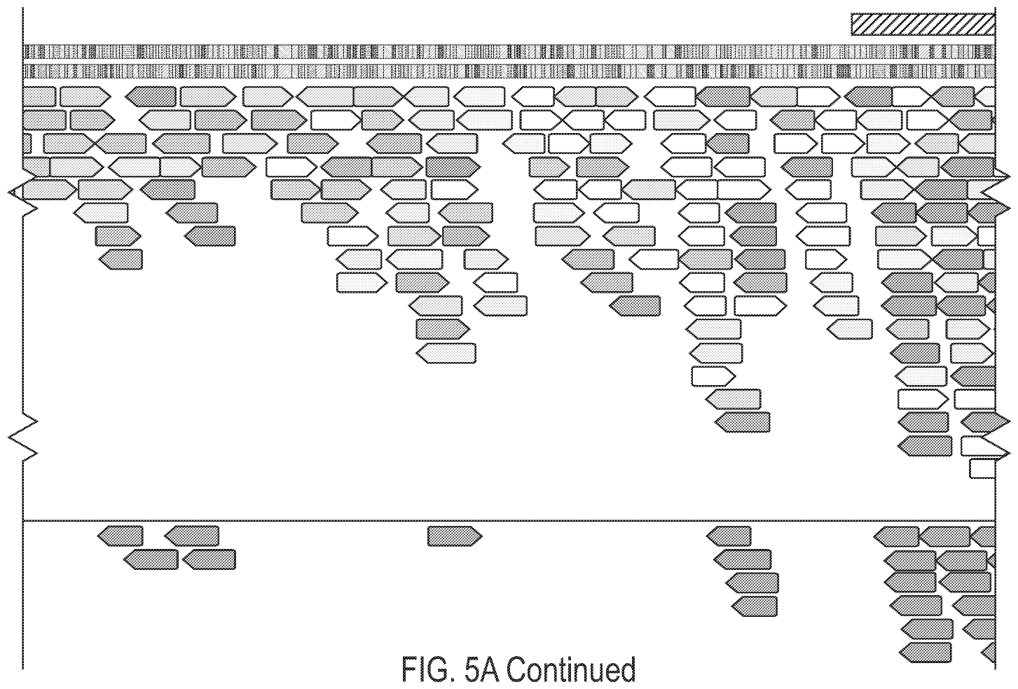

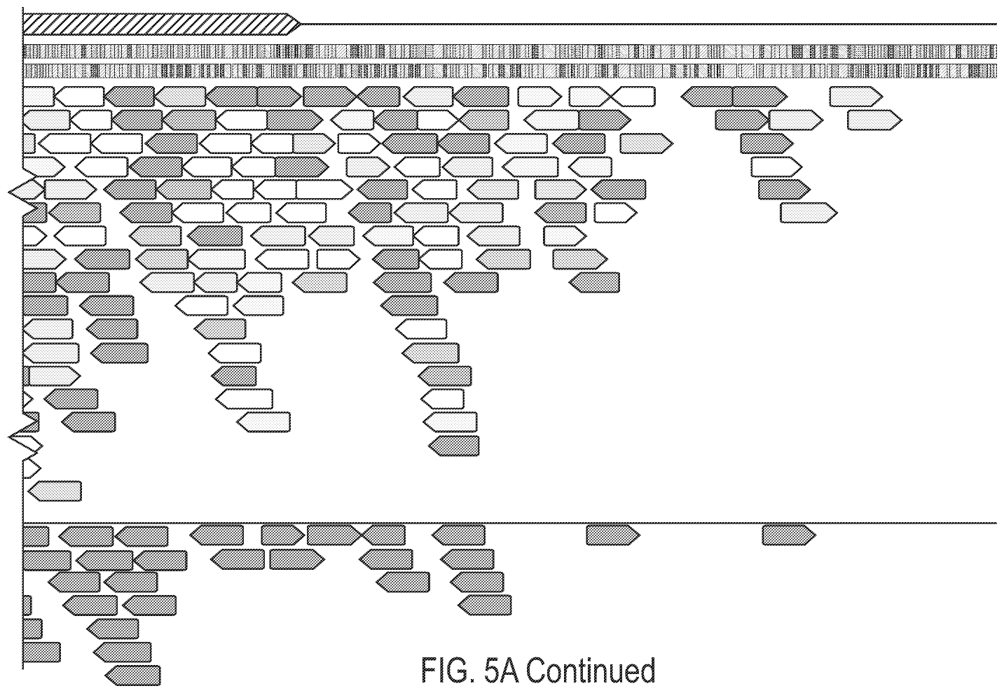

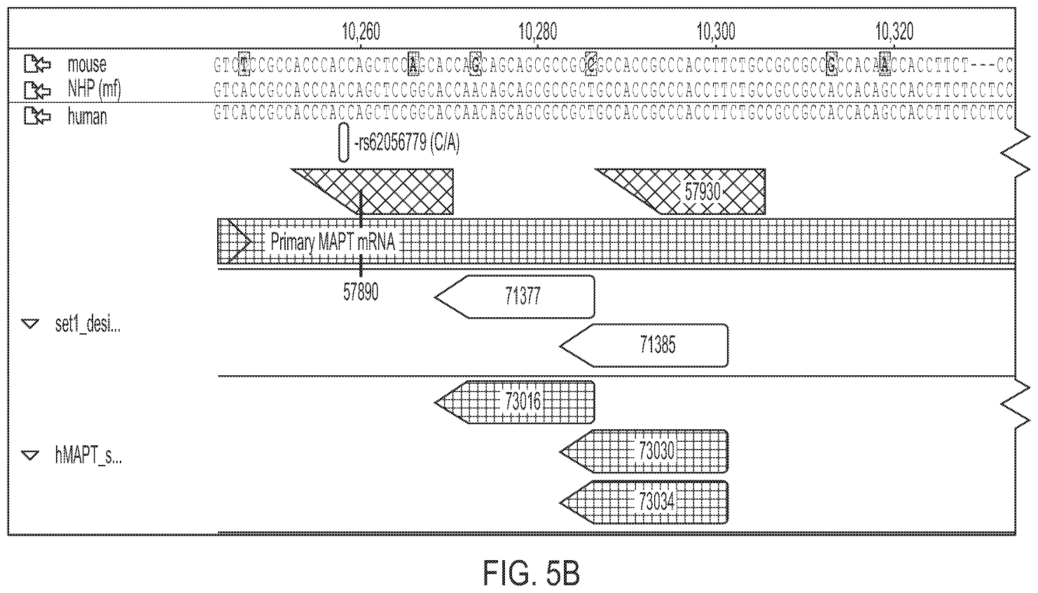

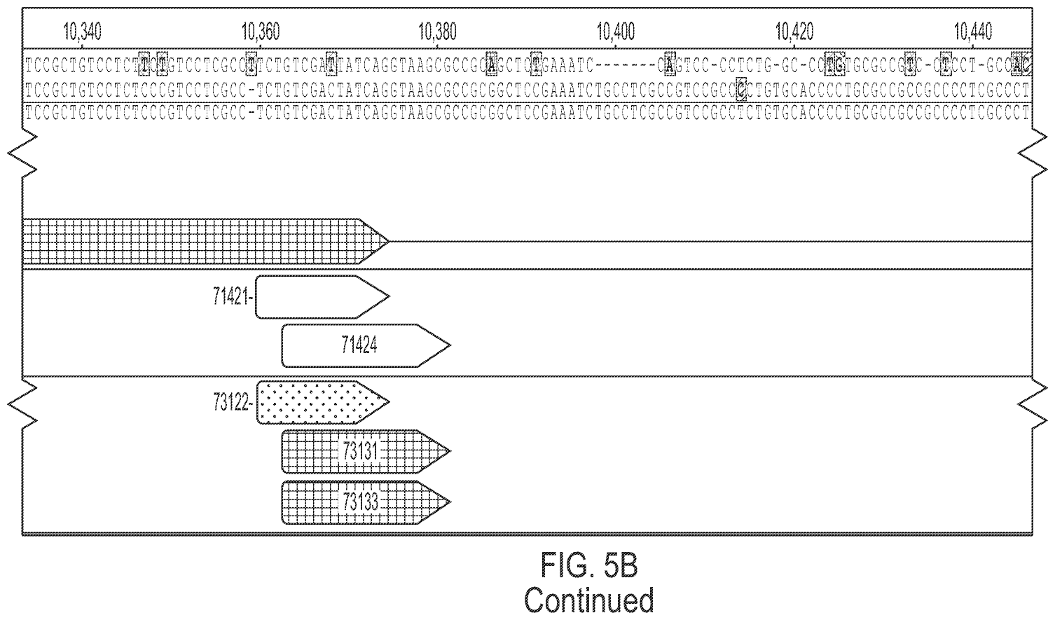

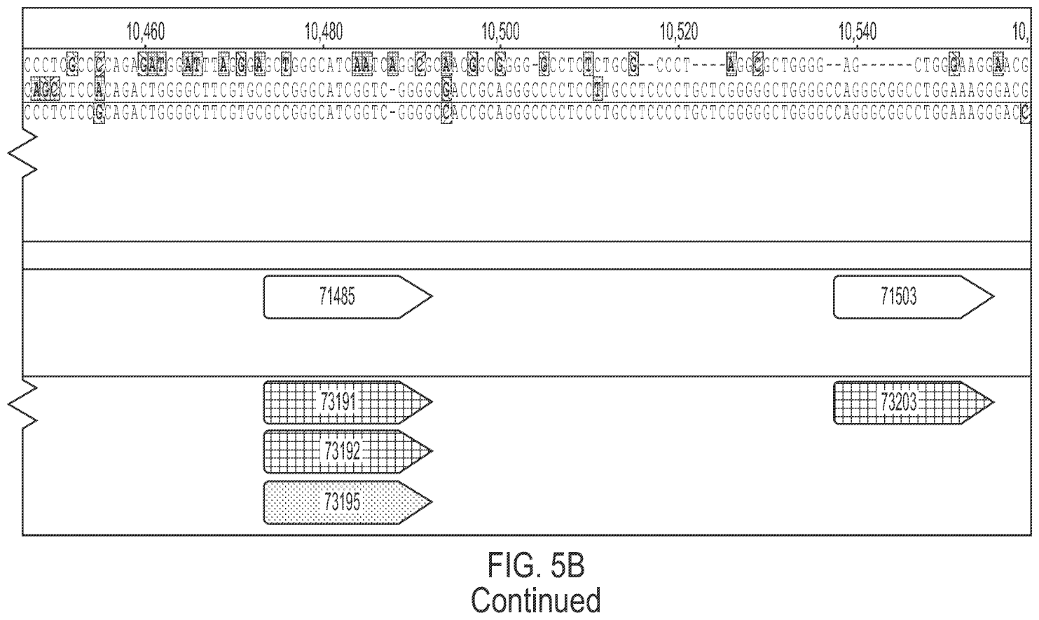

To optimize the target specificity of the ZFP-TFs, an arginine (R) residue in up to three of the zinc fingers was mutated to glutamine (Q). This arginine residue is located at the 4^{th} amino acid upstream of the 1^{st} amino acid in the DNA-binding helix and is in the β-sheet of each zinc finger (FIGS. 4, 5A and 5B). The residue is involved in a conserved non-specific contact with the phosphate backbone of the target DNA. Each parent ZFP-TF was mutated at the 4th position upstream of the 1st amino acid in the indicated helix to generate up to 7 different R→Q variants. The sequences of 41 representative R→Q variant ZFP-TFs are shown in FIG. 16 below. See also Miller et al., Nat Biotechnol. (2019) 37:945-52.

### Example 3: Screening of the R→Q Variants of Parental Anti-Tau ZFP-TFs

In order to identify the R→Q variants of parental ZFP-TFs that best repress the expression of human tau, a library of about 340 variant ZFP-TFs was screened as described in Example 1. The tau repression activity of representative R→Q variant ZFP-TFs was also tested in human iPSC-derived neurons and primary mouse cortical neurons transduced with AAVs encoding the respective ZFP-TFs.

### AAV Production

Recombinant adeno-associated virus vectors (rAAV) were generated by the triple transfection method. Briefly, HEK293 cells were plated in ten-layer CellSTACK chambers (Corning, Acton, Mass.) and grown for three days to a density of 80%. Three plasmids—(i) an AAV Helper plasmid containing the Rep and Cap genes, (ii) an Adenovirus Helper plasmid containing the adenovirus helper genes, and (iii) a transgene plasmid containing the sequence to be packaged flanked by AAV2 inverted terminal repeats were transfected into the cells using calcium phosphate. After three days, the cells were harvested. The cells were then lysed by three rounds of freeze/thaw and the cell debris was removed by centrifugation. The rAAV was precipitated using polyethylene glycol. After resuspension, the virus was purified by ultracentrifugation overnight on a cesium chloride gradient. The virus was formulated by dialysis and then filter-sterilized. After adjusting the titer (virus genomes/ml) of all AAV batches by dilution with PBS+0.001% Pluronic F-68, the AAVs were aliquoted to single use doses and stored at −80° C. until use. After thawing, no refreezing was done.

Human iPSC-Derived Neuron Culture and ZFP-TF AAV Infection

Human iPSC-derived GABAergic neurons were purchased from Cellular Dynamics International and plated onto poly-L-ornithine- and laminin-coated 96-well plates at a density of 40,000 cells per well and maintained according to the manufacturer's instructions. The cells were infected with AAV expressing the desired ZFP-TF at the indicated MOI 48 hours after plating and maintained for up to 32 days (50-75% media changes performed every 3-5 days). The cells were harvested at the end of the experimental period, RNA was isolated, and RT-qPCR was performed for gene expression analysis. For microarray analysis, the cells were transfected with 1E5 VGs/cell 48 hours after plating and harvested 19 days after viral transfection.

### Primary Mouse Neuron Culture and ZFP-TF AAV Infection

Primary mouse cortical neurons (MCNs) were purchased from Gibco. Cells were plated onto poly-D-lysine-coated 96- or 24-well plates at 50,000 or 200,000 cells/well, respectively, and maintained according to the manufacturer's specifications using Gibco Neurobasal Medium containing GlutaMAX™ I supplement, B27 supplement, and penicillin/streptomycin. 48 h after plating (at DIV2), 50,000 cells/well in 96-well plates were infected with AAV-ZFP at the indicated MOIs and harvested 7 days later (at DIV9; 50% media exchanges performed every 3-4 days) followed by RNA isolation and gene expression analysis by RT-qPCR. Alternatively, 200,000 neurons/well in 24-well plates were treated with 1E5 VGs/cell to ensure a 100% transduction rate, and processed in a similar fashion for microarray analysis at DIV9.

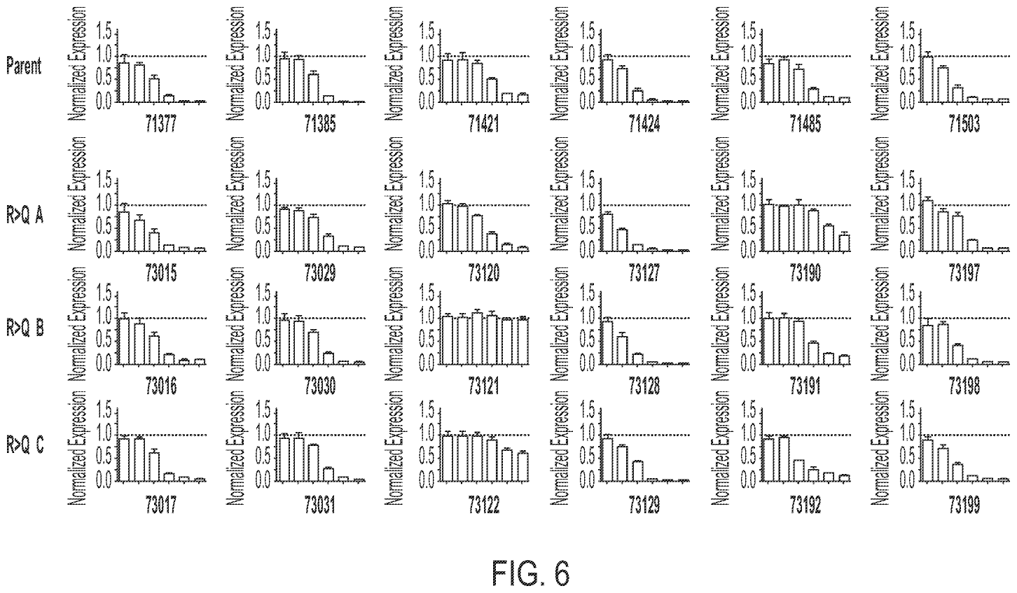

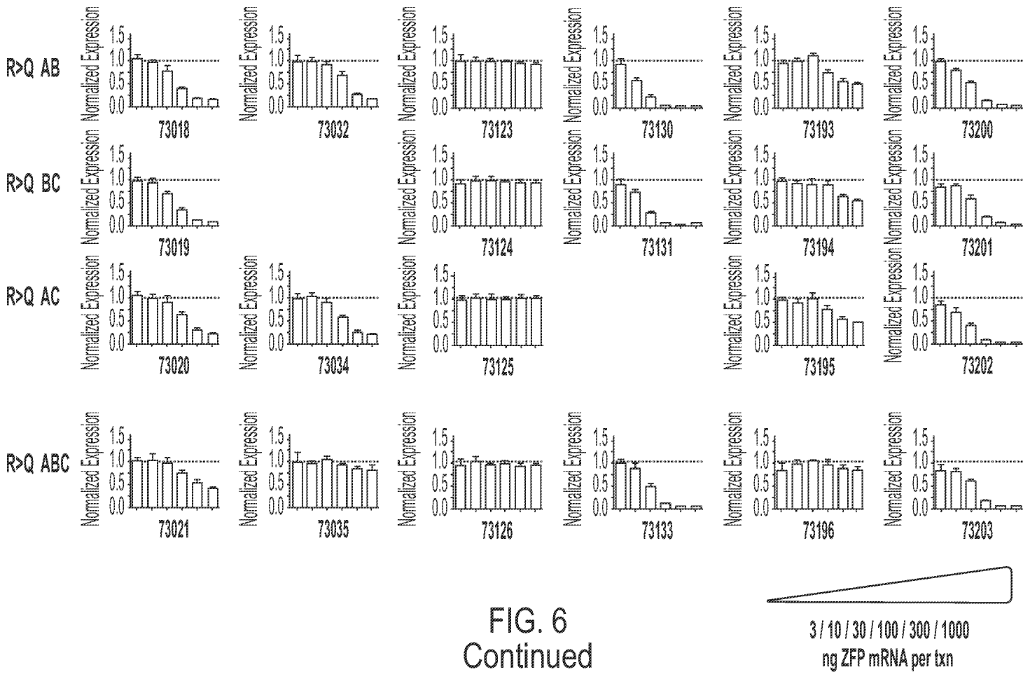

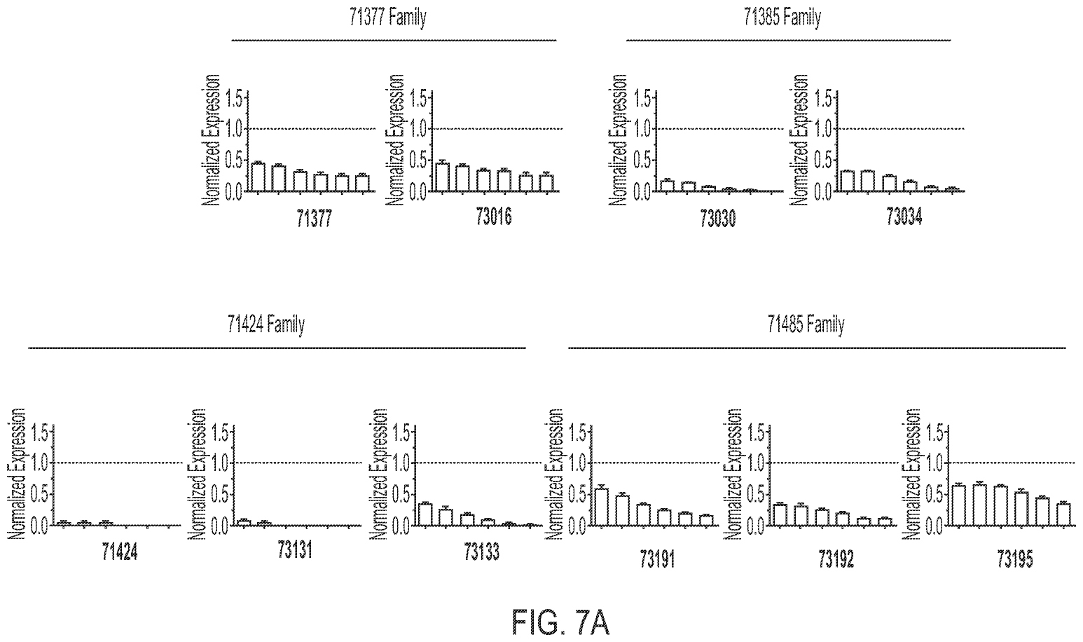

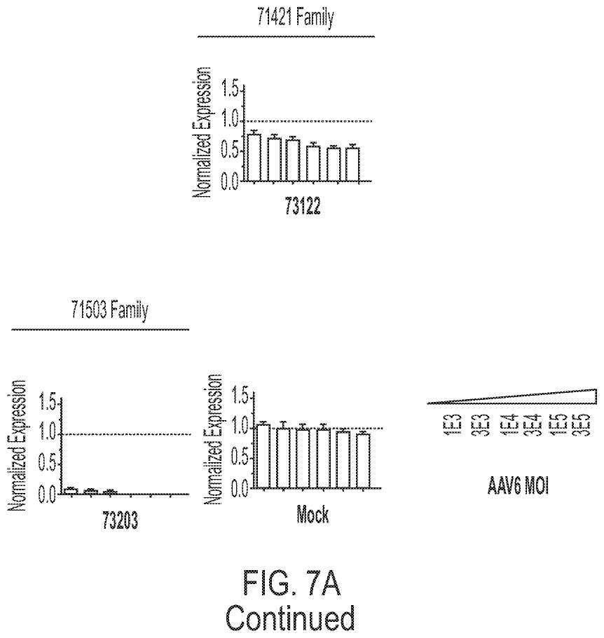

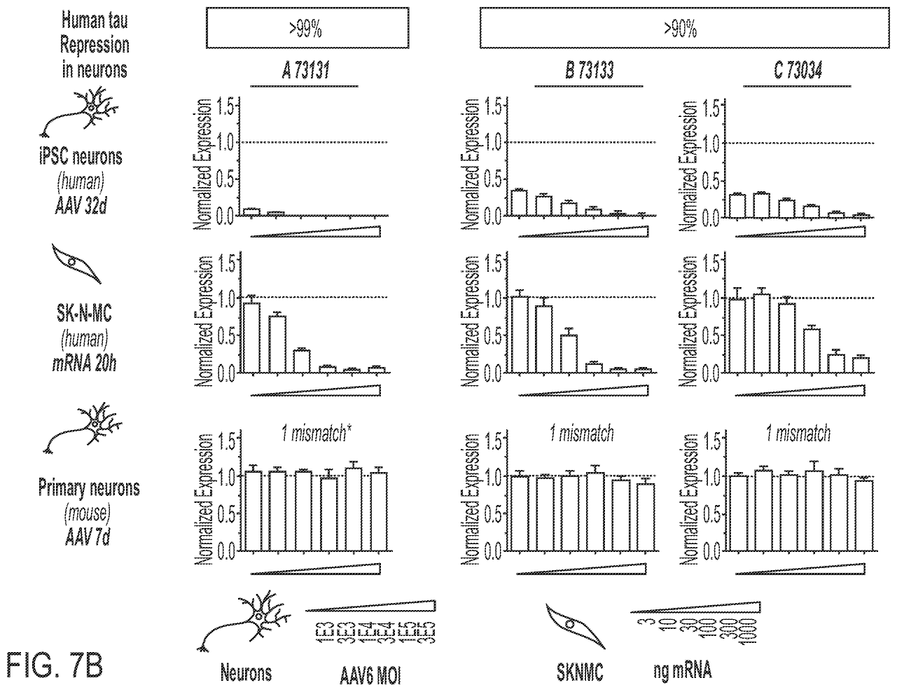

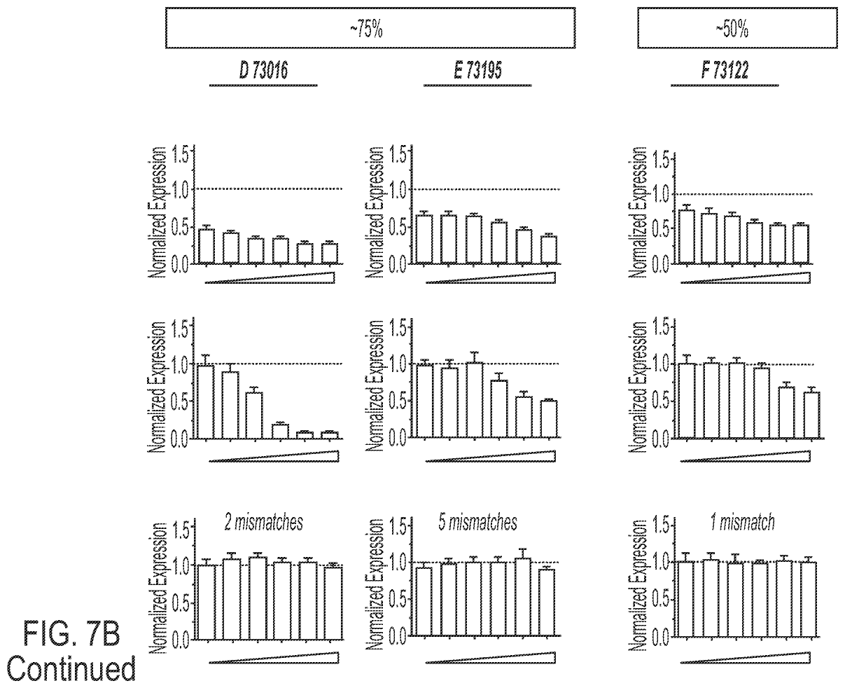

FIG. 6 shows the screening data of representative R→Q variant ZFP-TFs. FIGS. 7A and 7B show the dose-dependent activities of representative ZFP-TFs and their R→Q variants in human iPSC-derived neurons and mouse primary neurons transduced with AAVs encoding the respective ZFP-TFs.

The data in these figures show that the R→Q variant ZFP-TFs display a wide range of tau repression activity profiles, with human tau mRNA repression in human iPSC-derived neurons at the highest dose tested ranging from about 45% to 100%. It is also apparent that the tested ZFP-TFs specifically target human MAPT as expression of representative R→Q variant ZFP-TFs in primary cortical mouse neurons did not result in dose-dependent repression of total mouse Mapt after 7 days.

### Example 4: Off-Target Activity of Anti-Tau ZFP-TFs

To evaluate the off-target impact of the R→Q variants of parental ZFP-TFs on global gene expression, we performed microarray (Clariom S Array and Clariom D Array) experiments on total RNA isolated from human iPSC-derived neurons and primary mouse cortical neurons treated with AAVs encoding representative R→Q variant ZFP-TFs. We also performed quantitative RT-qPCR analyses to compare the effects of these ZFP-TFs on the expression levels of transcripts within the tau locus (human tau and STH) and apparent off-target genes (CPNE6 and IGF2) identified in the microarray studies.

### Microarray Analyses

Microarray analyses were performed following the manufacturer's protocol (Thermo Fisher Scientific), and the assay results were analyzed using TAC4 software. Apparent off-targets with FDR-corrected p-values≤0.05 were further investigated using RT-qPCR analysis.

### Gene Expression Analysis Using RT-qPCR

Reverse transcription was performed using the C2CT kit following the manufacturer's instructions. TaqMan quantitative polymerase chain reaction (qPCR) was used to measure the transcript levels of Mapt, Sth, Cpne6, and Igf2. Gene expression levels were normalized to the geometric mean of the expression levels of the housekeeping genes Atp5b, Eif4a2, and Gapdh. A mock transfection and transfection with a ZFP-TF known not to target MAPT, STH, CPNE6, and IGF2 were used as negative controls.

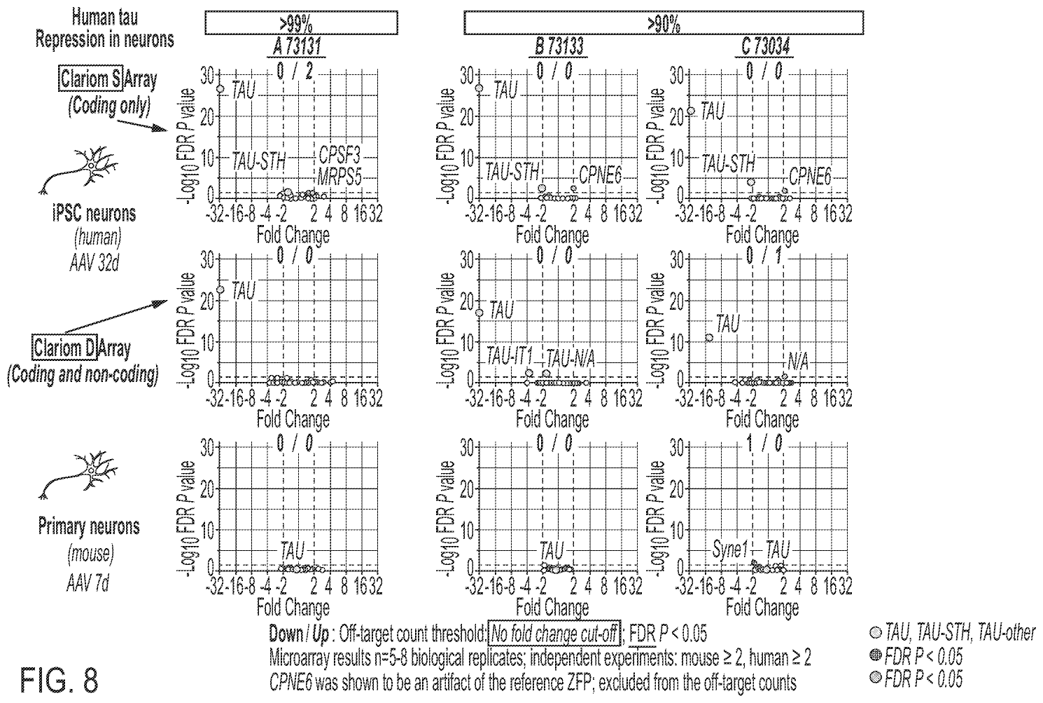

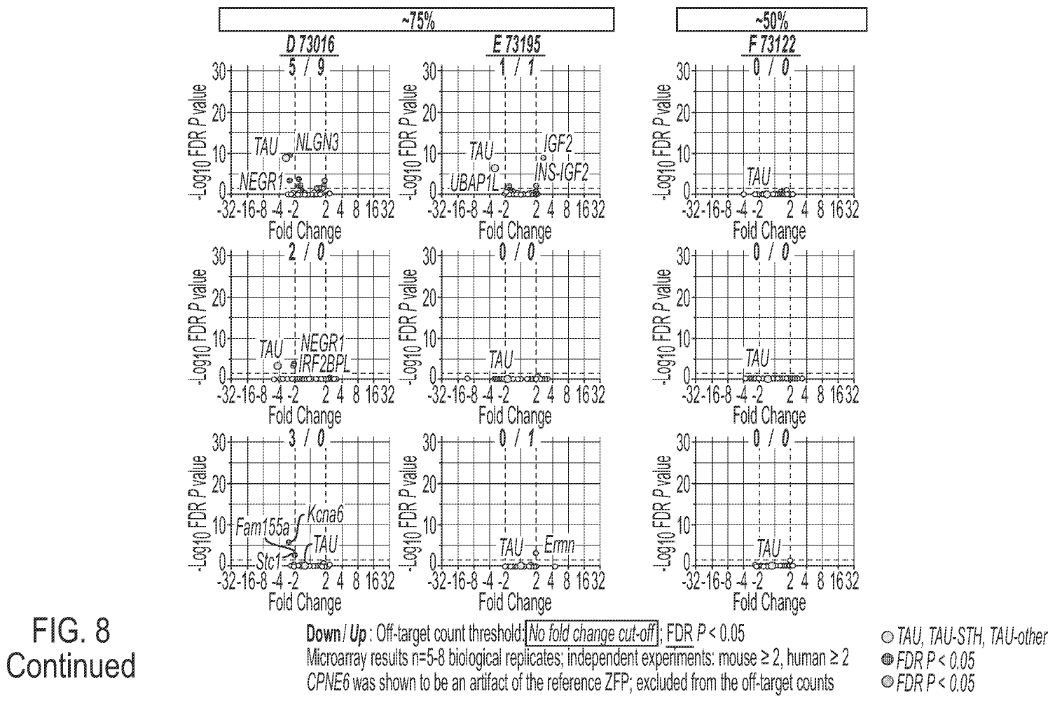

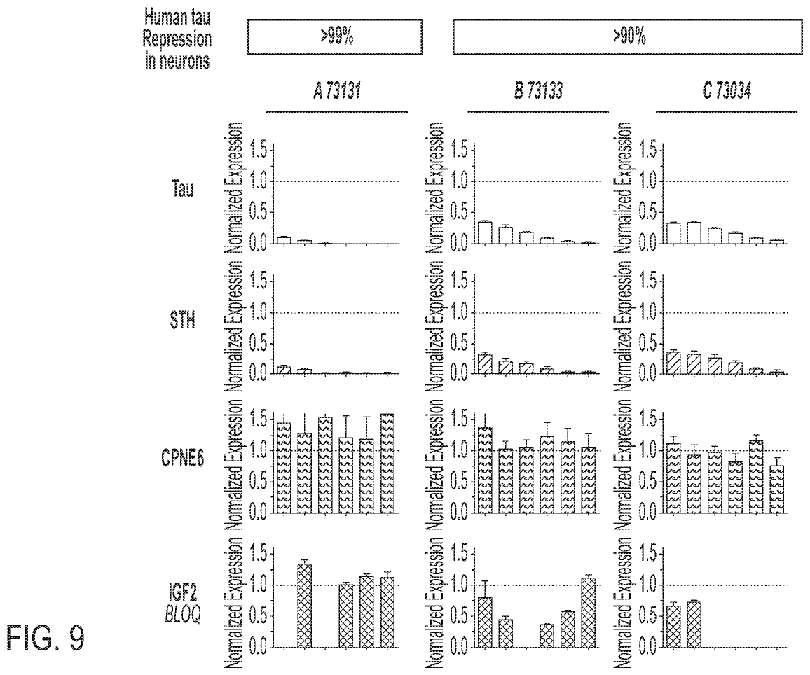

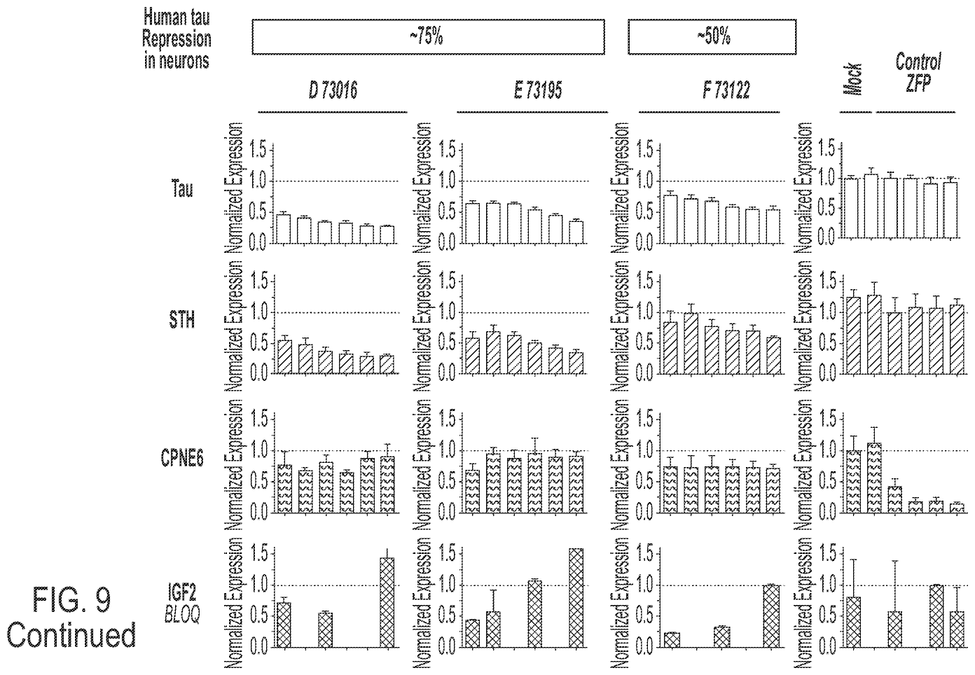

FIG. 8 shows the microarray results of 6 representative R→Q variant ZFP-TFs in human iPSC-derived neurons and primary mouse cortical neurons. FIG. 9 shows the RT-qPCR results for Mapt, Sth, Cpne6, and Igf2 gene expression in human iPSC-derived neurons transduced with representative R→Q variant ZFP-TFs.

We found that R→Q variant ZFP-TFs display low to no detectable off-target activity.

### Example 5: In Vivo Tolerability of Representative R→Q Variants of Anti-Tau ZFP-TFs

To evaluate potential adverse effects of ZFP-TF-mediated tau repression following intrahippocampal stereotaxic AAV9 administration, we assessed transgene expression and the expression levels of neuroinflammatory markers through quantitative RT-qPCR analyses of ZFP-TF, tau, Gfap, Iba1, and NeuN expression in hippocampal tissue isolated from the brains of adult C57BL/6 mice 4 weeks after treatment with AAVs encoding R→Q variants of parent ZFP-TFs with a range of human tau repression activity.

To harvest the hippocampal tissue for subsequent analysis, mice were perfused with PBS, and the brain was extracted and dissected on ice. Hippocampi were minced with a razor blade or scalpel and the minced tissue was divided into two parts designated for RNA and DNA analysis. The minced tissue was then flash-frozen in liquid nitrogen and maintained at −80° C. until analysis.

Reverse transcription was performed using the High Capacity RT Kit (Thermo Fisher Scientific) kit following the manufacturer's instructions. TaqMan quantitative polymerase chain reaction (qPCR) was used to measure the expression levels of the ZFP-TFs, Mapt, Gfap, Iba1, and NeuN. Gene expression levels were normalized to the geometric mean of the expression levels of the housekeeping genes Atp5b, Eif4a2, and Gapdh. Vehicle treatment and treatment with an AAV expression green fluorescent protein (GFP) were used as negative controls.

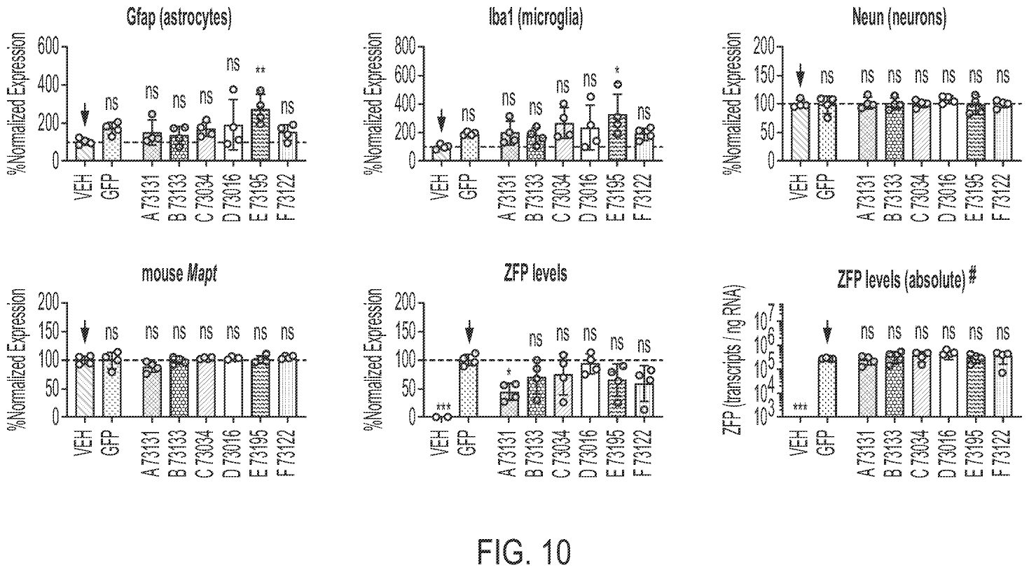

We found that most of the tested R→Q variant ZFP-TFs were well tolerated in vivo at the maximal administered dose. Several candidates resulted in no significant changes in the expression levels of neuroinflammatory markers. Further, several candidates showed no cross-reactivity to mouse tau, indicating a high degree of specificity. Adult mice treated with representative R→Q variant ZFP-TFs displayed stable mouse Mapt expression, thereby showing that these ZFP-TFs specifically target the human MAPT gene. One fusion protein, however, exhibited reduced expression of the ZFP-TFs and another fusion protein led to elevations in the expression levels of the neuroinflammatory markers Gfap and Iba1. None of the ZFP-TFs tested led to a decrease in the expression level of the neuronal marker NeuN. These findings indicate that the tau ZFP-TF candidates tested in this study have a desirable profile in vivo in mice, with no to minimal evidence of neuroinflammatory marker elevation and no neuronal loss following expression in the mouse brain (FIG. 10).

### Example 6: In Vivo Reduction of Human Tau mRNA and Protein by Representative R→Q Variants of Anti-Tau ZFP-TFs

To evaluate reduction of human tau mRNA and protein in vivo, anti-Tau ZFP-TFs were administered to htau mice (B6.Cg-Mapt^{tm1(EGFP)Kit}Tg(MAPT)8cPdav/J, Jackson Labs) as described herein. htau mice express the wild-type human MAPT gene on a background with the endogenous (mouse) Mapt functionally knocked-out by insertion of a GFP-expressing construct into the first coding exon of mouse Mapt. Mice received stereotaxic dual, bilateral injections into the dorsal and ventral hippocampus of AAV9 vectors encoding either vehicle, 73133, 73034, 73122, or 65918.T2A.57890 (a construct that co-expresses two ZFPs designed to target sites in the mouse Mapt gene). ZFP-TFs 73133, 73034, and 73122 were tested at doses of 3E9, 1E0, and 3E10 VG per hemisphere, whereas ZFP-TF 65918.T2A.57890 was tested at only the 3E9 VG per hemisphere. Separate groups of mice were sacrificed at either 3 mo or 6 mo after injection for molecular, biochemical, and immunohistological endpoints.

The expression levels of ZFP transgene, human MAPT, endogenous mouse Mapt, and the GFP cassette that disrupts endogenous Mapt translation were assessed by quantitative RT-qPCR in hippocampal tissue isolated from the brains of htau mice. Neuroinflammatory and neuronal markers were also assessed, including Gfap, Iba1, and NeuN. In addition, the levels of an intronic transcript of unknown function within human MAPT named Saitohin (STH) were evaluated.

To harvest the hippocampal tissue for subsequent analysis, mice were perfused with PBS, and the brain was extracted and dissected on ice. For the right hemisphere, hippocampi were minced with a razor blade or scalpel and the minced tissue was divided into two parts designated for RNA and protein analysis. The minced tissue was then flash-frozen in liquid nitrogen and maintained at −80° C. until analysis. For the left hemisphere, hippocampi were drop-fixed in 10% NBF for 24 hours, transferred to 70% ethanol, then embedded in paraffin blocks for subsequent in situ hybridization (ISH) analysis.

For mRNA analysis, total RNA was extracted using the MagMax for microarray RNA extraction kit (Thermo Fisher Scientific) and reverse transcription was performed using the High Capacity RT Kit (Thermo Fisher Scientific) kit following the manufacturer's instructions. TaqMan™ quantitative polymerase chain reaction (qPCR) was used to measure target gene expression level. ZFP-transgene expression was normalized to amount of total RNA used as input for the RT-qPCR reaction. All other target gene expression levels were normalized to the geometric mean of the expression levels of the housekeeping genes Atp5b, Eif4a2, and Gapdh. All treatment groups were scaled to the mean of the normalized levels observed for the vehicle group.

For protein analysis, a total human tau ELISA (Thermo Fisher Scientific) kit was used to quantify the levels of human tau protein following the manufacturer's protocol. Total tau levels were normalized to total protein as determined by a BCA protein assay. All treatment groups were scaled to the mean of the normalized levels observed for the vehicle group.

For ISH analysis, paraffin embedded blocks were sectioned and treated for multiplexed RNAscope/immunofluorescence staining according to the manufacture's protocols (Advanced Cell Diagnostics). Sections were stained for nuclei with DAPI, human MAPT transcripts by RNAscope, and NeuN protein by immunofluorescence.

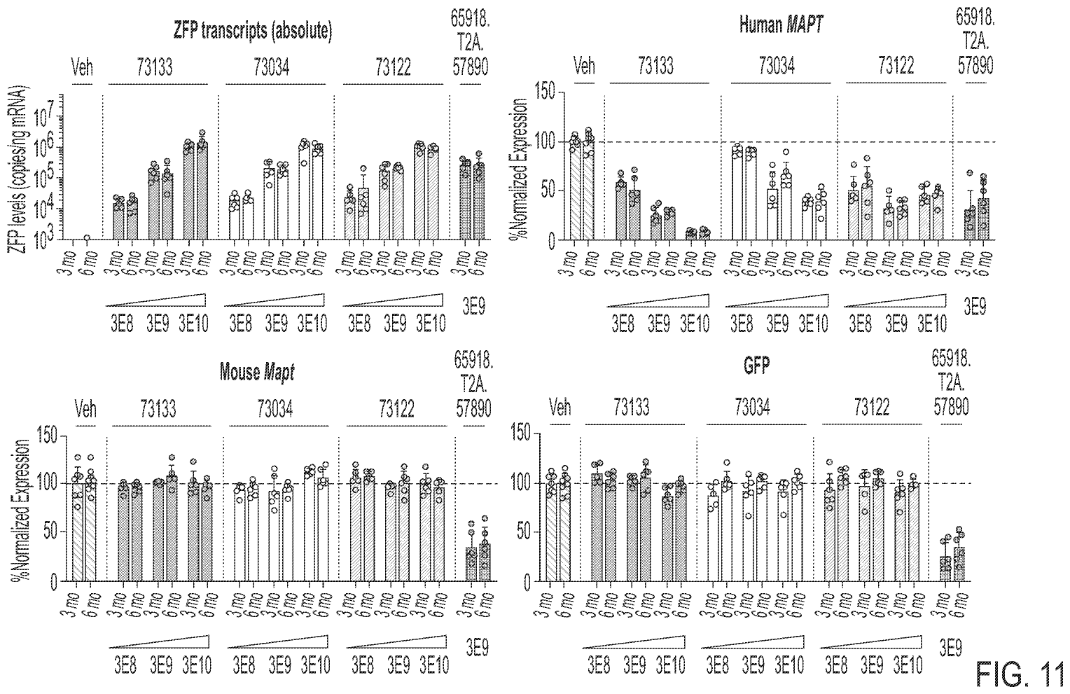

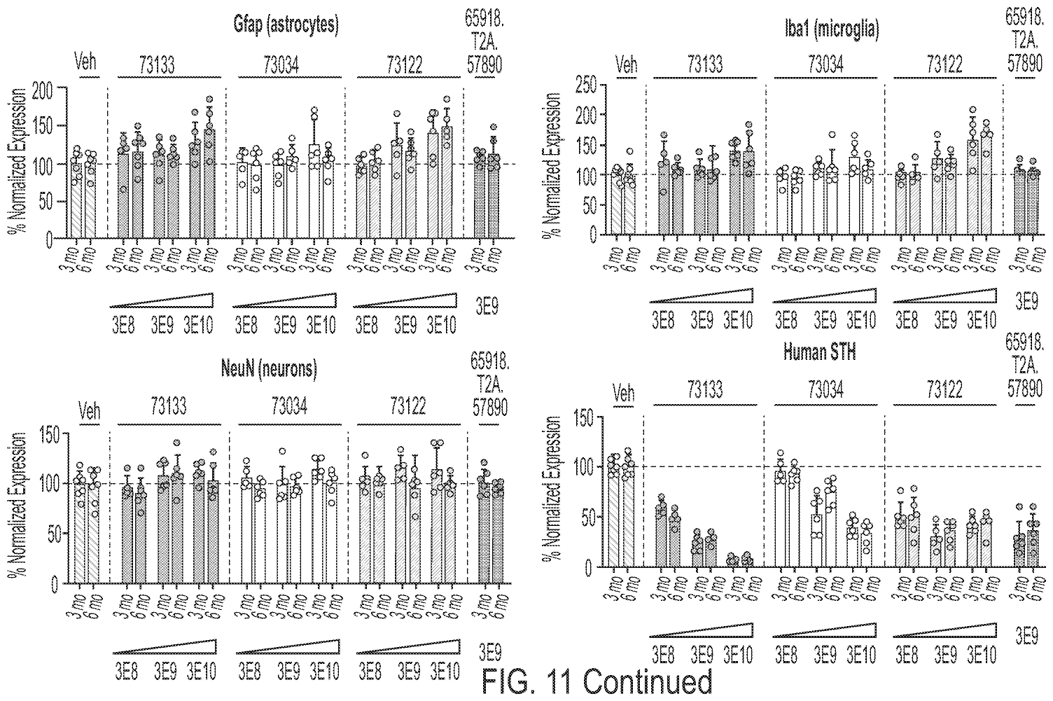

Each of the ZFP-TFs was expressed in a persistent, dose-dependent manner, and at similar levels across all constructs tested for each corresponding dose at both the 3-month and 6-month time points. The human targeted anti-tau ZFP-TFs were capable of specifically reducing human tau mRNA by up to 93% (73133), or to 54-61% (73034 and 73122) at the highest dose tested at both time points. ZFP-TFs 73133 and 73034 resulted in a dose-dependent reduction of tau mRNA, whereas 73122 resulted in a plateaued effect across the range of tested doses (˜46-68% reduction). Similar results were observed for the intronic MAPT transcript STH for all ZFP-TFs and timepoints tested. For 73133, 73034, and 73034 there was no significant reduction of endogenous mouse Mapt or GFP expression. The cross-species targeting 65918.T2A.57890 did reduce both human and mouse tau by >50% at both time points at the single dose tested (3E9 VG per hemisphere). No or minimally significant changes in the expression levels of neuroinflammatory and neuronal markers Gfap, Iba1, or Neun were observed in the ZFP-treated groups (FIG. 11).

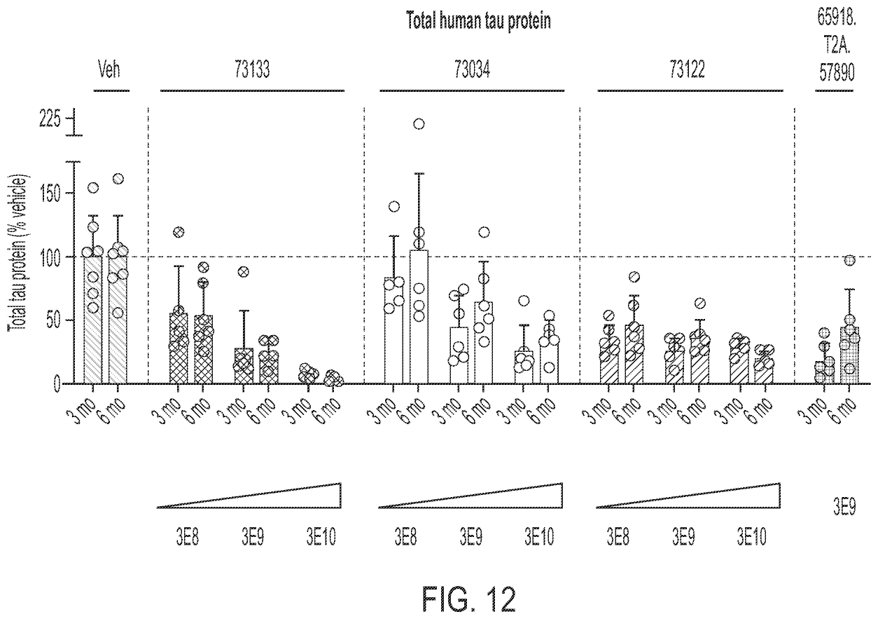

The human targeted anti-tau ZFP-TFs similarly reduced human tau protein levels up to 97% (73133) to >64-80% (73034 and 73122) at the highest dose tested at both time points. ZFP-TFs 73133 and 73034 resulted in a dose-dependent reduction of tau protein, whereas 73122 resulted in a plateaued effect across the range of tested doses (˜54-80% reduction; FIG. 12).

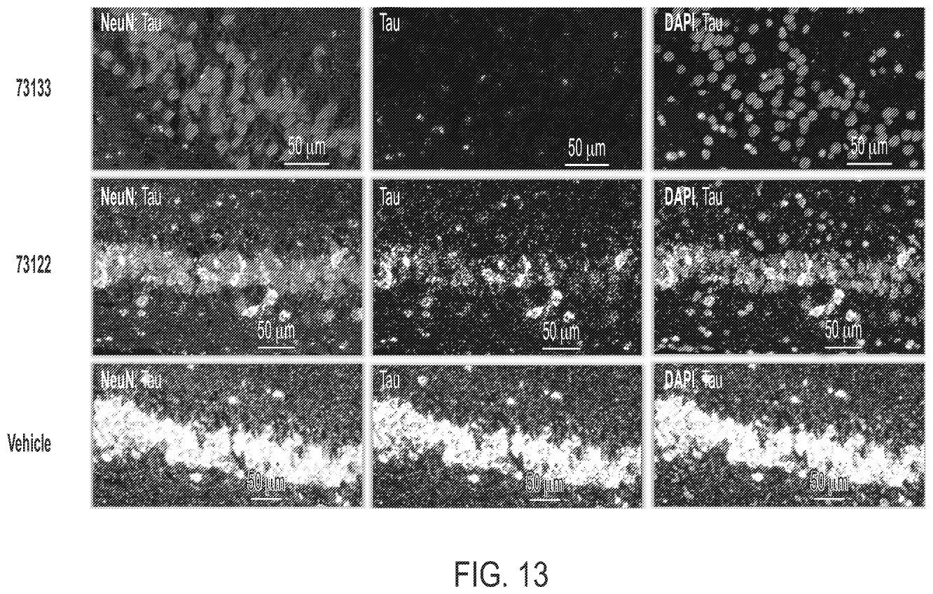

RNAscope analysis showed a ZFP-dependent degree of human MAPT transcript reduction in the hippocampus of treated htau mice. Compared to vehicle-treated mice, NeuN positive neurons in hippocampi treated with ZFP-TF 73133 had minimal detectable MAPT transcript remaining at three months at the 3e9 tested dose. In contrast, 73122 had intermediate levels of detectable MAPT transcript remaining, consistent with the bulk MAPT mRNA and tau protein analysis showing less total repression for this ZFP-TF (FIG. 13). These single-cell data support the repression results obtained for MAPT targeted ZFP-TFs expressed in SK-N-MC cells, cultured human iPSC-derived neurons, and bulk brain tissue from htau mice.

These findings indicate that the tau ZFP-TF candidates tested in this study have desirable tau-lowering profiles in vivo in mice, with minimal evidence of neuroinflammatory marker elevation and no neuronal loss following expression in the htau mouse brain.

1. A fusion protein comprising a zinc finger protein (ZFP) domain and a transcription repressor domain, wherein the ZFP domain binds to a target region of a human microtubule-associated protein tau (MAPT) gene.

2. The fusion protein of claim 1, wherein the target region is within 1.5 kb of a transcription start site (TSS) in the MAPT gene.

3. The fusion protein of claim 2, wherein the target region is within 1000 bps upstream of the TSS, and/or within 500 bps downstream of the TSS of the MAPT gene.

4. The fusion protein of claim 1, wherein the fusion protein represses expression of the MAPT gene by at least about 40%, 75%, 90%, 95%, or 99% with no or minimal detectable off-target binding or activity.

5. The fusion protein of claim 1, wherein the transcription repressor domain comprises a KRAB domain, wherein the KRAB domain optionally is from a human KOX1 protein.

6. The fusion protein of claim 1, wherein the DNA-binding domain is linked to the transcription repressor through a peptide linker.

7. The fusion protein of claim 1, wherein the ZFP domain comprises a DNA-binding recognition helix sequence shown in FIG. 14 or FIG. 16.

8. The fusion protein of claim 1, wherein the ZFP domain comprises the DNA-binding recognition helix sequences as shown in a single row of FIG. 14 or FIG. 16.

9. The fusion protein of claim 1, wherein the ZFP domain of the fusion protein
comprises four, five, or six zinc fingers;
binds to a target sequence shown in FIG. 14 or FIG. 16;
comprises the DNA-binding recognition helix sequences of a ZFP transcription factor shown in FIG. 15 or FIG. 17;
comprises the DNA-binding recognition helix sequences linked as shown in FIG. 14, FIG. 15, FIG. 16, or FIG. 17; and/or
comprises an amino acid sequence selected from SEQ ID NOs: 89-196, 197-248 and 267-307.

10. A nucleic acid construct comprising a coding sequence for the fusion protein of claim 1, wherein the coding sequence is linked operably to a transcription regulatory element.

11. The nucleic acid construct of claim 10, wherein the transcription regulatory element is a mammalian promoter that is constitutively active or inducible in a brain cell, wherein the construct is optionally a recombinant viral construct.

12. A recombinant virus comprising the nucleic acid construct of claim 10.

13. The recombinant virus of claim 12, wherein the recombinant virus is an adeno-associated viral vector, an adenoviral vector, or a lentiviral vector.

14. A pharmaceutical composition comprising the nucleic acid construct of claim 10 and a pharmaceutically acceptable carrier.

15. A host cell comprising the nucleic acid construct of claim 10.

16. The host cell of claim 15, wherein the host cell is a human cell.

17. The host cell of claim 15 or 16, wherein the host cell is a brain cell or a pluripotent stem cell, wherein the stem cell is optionally an embryonic stem cell or an inducible pluripotent stem cell (iPSC).

18. A method of inhibiting expression of tau in a human brain cell, comprising introducing into the cell a fusion protein of claim 1, thereby inhibiting the expression of tau in the cell.

19. The method of claim 18, wherein the human brain cell is a neuron, a glial cell, an ependymal cell, a neuroepithelial cell, an endothelial cell, or an oligodendrocyte.

20. The method of claim 18, wherein the cell is in the brain of a patient suffering from or at risk of developing Alzheimer's disease, frontotemporal dementia, progressive supranuclear palsy, traumatic brain injury (TBI), seizure disorders, corticobasal degeneration (CBD), chronic traumatic encephalopathy (CTE), or another tauopathy.

21. The method of claim 18, comprising introducing into the cell a recombinant virus that expresses the fusion protein.

22. The method of claim 21, wherein the recombinant virus is an adeno-associated virus (AAV), optionally of serotype 9 or a pseudotype derived from AAV9.

23. A method of treating a tauopathy in a patient in need thereof, comprising administering to the patient a recombinant AAV or a nucleic acid construct encoding a fusion protein of claim 1.

24. The method of claim 23, wherein the AAV or nucleic acid construct is introduced to the patient via an intravenous, intrathecal, intracerebral, intracerebroventricular, intra-cisternal magna, intrahippocampal, intrathalamic, or intraparenchymal route.

25. The method of claim 23, wherein the tauopathy is Alzheimer's disease, or frontotemporal dementia, progressive supranuclear palsy, traumatic brain injury (TBI), seizure disorders, corticobasal degeneration (CBD), or chronic traumatic encephalopathy (CTE).

26-27. (canceled)
